(19) United States
(12) Patent Application Publication
(10) Pub. No.: US 2024/0175057 A1
(43) Pub. Date: May 30, 2024

(54) RNA-Guided Transcriptional Regulation

(71) Applicant: President and Fellows of Harvard College, Cambridge, MA (US)

(72) Inventors: George M. Church, Brookline, MA (US); Prashant G. Mali, La Jolla, CA (US); Kevin M. Esvelt, Auburndale, MA (US)

(21) Appl. No.: 18/425,219

(22) Filed: Jan. 29, 2024

Related U.S. Application Data
(63) Continuation of application No. 17/972,885, filed on Oct. 25, 2022.
Application No. 17/972,885 is a continuation of application No. 16/851,360, filed on Apr. 17, 2020.
Application No. 16/851,360 is a continuation of application No. 16/441,209, filed on Jun. 14, 2019, now Pat. No. 10,767,194.
Application No. 16/441,209 is a continuation of application No. 14/319,530, filed on Jun. 30, 2014.
Application No. 14/319,530 is a continuation of application No. PCT/US2014/040868, filed on Jun. 4, 2014.
(60) Provisional application No. 61/830,787, filed on Jun. 4, 2013.

(51) Int. Cl. C12N 15/90; C12N 9/22; C12N 15/10; C12N 15/11; C12N 15/113; C12N 15/63

(52) U.S. Cl. CPC C12N 15/907; C12N 9/22; C12N 15/102; C12N 15/11; C12N 15/113; C12N 15/635; C12N 2310/20; C12N 2310/3513; C12Y 301/00

(57) ABSTRACT

Methods of modulating expression of a target nucleic acid in a cell are provided including introducing into the cell a first foreign nucleic acid encoding one or more RNAs complementary to DNA, wherein the DNA includes the target nucleic acid, introducing into the cell a second foreign nucleic acid encoding a nuclease-null Cas9 protein that binds to the DNA and is guided by the one or more RNAs, introducing into the cell a third foreign nucleic acid encoding a transcriptional regulator protein or domain, wherein the one or more RNAs, the nuclease-null Cas9 protein, and the transcriptional regulator protein or domain are expressed, wherein the one or more RNAs, the nuclease-null Cas9 protein and the transcriptional regulator protein or domain co-localize to the DNA and wherein the transcriptional regulator protein or domain regulates expression of the target nucleic acid.

### RELATED APPLICATION DATA

This application is a continuation of U.S. Pat. No. 17/972,885 and filed Oct. 25, 2022, which is a continuation of U.S. patent application Ser. No. 16/851,360 and filed Apr. 17, 2020, which is a continuation of U.S. patent application Ser. No. 16/441,209 and filed Jun. 14, 2019, which is a continuation of U.S. patent application Ser. No. 14/319,530, filed on Jun. 30, 2014, which is a continuation of PCT application no. PCT/US2014/040868, designating the United States and filed Jun. 4, 2014; which claims the benefit U.S. Provisional Patent Application No. 61/830,787 filed on Jun. 4, 2013; each of which are hereby incorporated by reference in their entireties.

### STATEMENT OF GOVERNMENT INTERESTS

This invention was made with government support under HG005550 awarded by National Institutes of Health (NIH) and under DE-FG02-02ER63445 awarded by U.S. Department of Energy (DOE). The government has certain rights in this invention.

### REFERENCE TO AN ELECTRONIC SEQUENCE LISTING

The instant application contains a Sequence Listing which has been submitted electronically in XML format and is hereby incorporated by reference in its entirety. Said XML copy, created on Nov. 27, 2023, is named “Corrected Sequence_Listing_010498_01504_ST26” and is 251 KB in size.

### BACKGROUND

Bacterial and archaeal CRISPR-Cas systems rely on short guide RNAs in complex with Cas proteins to direct degradation of complementary sequences present within invading foreign nucleic acid. See Deltcheva, E. et al. CRISPR RNA maturation by trans-encoded small RNA and host factor RNase III. Nature 471, 602-607 (2011); Gasiunas, G., Barrangou, R., Horvath, P. & Siksnys, V. Cas9-crRNA ribonucleoprotein complex mediates specific DNA cleavage for adaptive immunity in bacteria. Proceedings of the National Academy of Sciences of the United States of America 109, E2579-2586 (2012); Jinek, M. et al. A programmable dual-RNA-guided DNA endonuclease in adaptive bacterial immunity. Science 337, 816-821 (2012); Sapranauskas, R. et al. The Streptococcus thermophilus CRISPR/Cas system provides immunity in Escherichia coli. Nucleic acids research 39, 9275-9282 (2011); and Bhaya, D., Davison, M. & Barrangou, R. CRISPR-Cas systems in bacteria and archaea: versatile small RNAs for adaptive defense and regulation. Annual review of genetics 45, 273-297 (2011). A recent in vitro reconstitution of the S. pyogenes type II CRISPR system demonstrated that crRNA (“CRISPR RNA”) fused to a normally trans-encoded tracrRNA (“trans-activating CRISPR RNA”) is sufficient to direct Cas9 protein to sequence-specifically cleave target DNA sequences matching the crRNA. Expressing a gRNA homologous to a target site results in Cas9 recruitment and degradation of the target DNA. See H. Deveau et al., Phage response to CRISPR-encoded resistance in Streptococcus thermophilus. Journal of Bacteriology 190, 1390 (February 2008).

### SUMMARY

Aspects of the present disclosure are directed to a complex of a guide RNA, a DNA binding protein and a double stranded DNA target sequence. According to certain aspects, DNA binding proteins within the scope of the present disclosure include a protein that forms a complex with the guide RNA and with the guide RNA guiding the complex to a double stranded DNA sequence wherein the complex binds to the DNA sequence. This aspect of the present disclosure may be referred to as co-localization of the RNA and DNA binding protein to or with the double stranded DNA. In this manner, a DNA binding protein-guide RNA complex may be used to localize a transcriptional regulator protein or domain at target DNA so as to regulate expression of target DNA.

According to certain aspects, a method of modulating expression of a target nucleic acid in a cell is provided including introducing into the cell a first foreign nucleic acid encoding one or more RNAs (ribonucleic acids) complementary to DNA (deoxyribonucleic acid), wherein the DNA includes the target nucleic acid, introducing into the cell a second foreign nucleic acid encoding an RNA guided nuclease-null DNA binding protein that binds to the DNA and is guided by the one or more RNAs, introducing into the cell a third foreign nucleic acid encoding a transcriptional regulator protein or domain, wherein the one or more RNAs, the RNA guided nuclease-null DNA binding protein, and the transcriptional regulator protein or domain are expressed, wherein the one or more RNAs, the RNA guided nuclease-null DNA binding protein and the transcriptional regulator protein or domain co-localize to the DNA and wherein the transcriptional regulator protein or domain regulates expression of the target nucleic acid.

According to one aspect, the foreign nucleic acid encoding an RNA guided nuclease-null DNA binding protein further encodes the transcriptional regulator protein or domain fused to the RNA guided nuclease-null DNA binding protein. According to one aspect, the foreign nucleic acid encoding one or more RNAs further encodes a target of an RNA-binding domain and the foreign nucleic acid encoding the transcriptional regulator protein or domain further encodes an RNA-binding domain fused to the transcriptional regulator protein or domain.

According to one aspect, the cell is a eukaryotic cell. According to one aspect, the cell is a yeast cell, a plant cell or an animal cell. According to one aspect, the cell is a mammalian cell.

According to one aspect, the RNA is between about 10 to about 500 nucleotides. According to one aspect, the RNA is between about 20 to about 100 nucleotides.

According to one aspect, the transcriptional regulator protein or domain is a transcriptional activator. According to one aspect, the transcriptional regulator protein or domain upregulates expression of the target nucleic acid. According to one aspect, the transcriptional regulator protein or domain upregulates expression of the target nucleic acid to treat a disease or detrimental condition. According to one aspect, the target nucleic acid is associated with a disease or detrimental condition.

According to one aspect, the one or more RNAs is a guide RNA. According to one aspect, the one or more RNAs is a tracrRNA-crRNA fusion. According to one aspect, the guide RNA includes a spacer sequence and a tracer mate sequence. The guide RNA may also include a traer sequence, a portion of which hybridizes to the tracr mate sequence. The guide RNA may also include a linker nucleic acid sequence which links the tracer mate sequence and the tracr sequence to produce the tracrRNA-crRNA fusion. The spacer sequence binds to target DNA, such as by hybridization.

According to one aspect, the guide RNA includes a truncated spacer sequence. According to one aspect, the guide RNA includes a truncated spacer sequence having a 1 base truncation at the 5′ end of the spacer sequence. According to one aspect, the guide RNA includes a truncated spacer sequence having a 2 base truncation at the 5′ end of the spacer sequence. According to one aspect, the guide RNA includes a truncated spacer sequence having a 3 base truncation at the 5′ end of the spacer sequence. According to one aspect, the guide RNA includes a truncated spacer sequence having a 4 base truncation at the 5′ end of the spacer sequence. Accordingly, the spacer sequence may have a 1 to 4 base truncation at the 5′ end of the spacer sequence.

According to certain embodiments, the spacer sequence may include between about 16 to about 20 nucleotides which hybridize to the target nucleic acid sequence. According to certain embodiments, the spacer sequence may include about 20 nucleotides which hybridize to the target nucleic acid sequence.

According to certain aspects, the linker nucleic acid sequence may include between about 4 and about 6 nucleic acids.

According to certain aspects, the tracr sequence may include between about 60 to about 500 nucleic acids. According to certain aspects, the tracr sequence may include between about 64 to about 500 nucleic acids. According to certain aspects, the tracr sequence may include between about 65 to about 500 nucleic acids. According to certain aspects, the traer sequence may include between about 66 to about 500 nucleic acids. According to certain aspects, the traer sequence may include between about 67 to about 500 nucleic acids. According to certain aspects, the traer sequence may include between about 68 to about 500 nucleic acids. According to certain aspects, the tracr sequence may include between about 69 to about 500 nucleic acids. According to certain aspects, the tracr sequence may include between about 70 to about 500 nucleic acids. According to certain aspects, the traer sequence may include between about 80 to about 500 nucleic acids. According to certain aspects, the tracr sequence may include between about 90 to about 500 nucleic acids. According to certain aspects, the traer sequence may include between about 100 to about 500 nucleic acids.

According to certain aspects, the traer sequence may include between about 60 to about 200 nucleic acids. According to certain aspects, the tracr sequence may include between about 64 to about 200 nucleic acids. According to certain aspects, the traer sequence may include between about 65 to about 200 nucleic acids. According to certain aspects, the tracr sequence may include between about 66 to about 200 nucleic acids. According to certain aspects, the traer sequence may include between about 67 to about 200 nucleic acids. According to certain aspects, the tracr sequence may include between about 68 to about 200 nucleic acids. According to certain aspects, the traer sequence may include between about 69 to about 200 nucleic acids. According to certain aspects, the tracr sequence may include between about 70 to about 200 nucleic acids. According to certain aspects, the tracr sequence may include between about 80 to about 200 nucleic acids. According to certain aspects, the traer sequence may include between about 90 to about 200 nucleic acids. According to certain aspects, the traer sequence may include between about 100 to about 200 nucleic acids.

An exemplary guide RNA is depicted in FIG. 5B.

According to one aspect, the DNA is genomic DNA, mitochondrial DNA, viral DNA, or exogenous DNA.

According to certain aspects, a method of modulating expression of a target nucleic acid in a cell is provided including introducing into the cell a first foreign nucleic acid encoding one or more RNAs (ribonucleic acids) complementary to DNA (deoxyribonucleic acid), wherein the DNA includes the target nucleic acid, introducing into the cell a second foreign nucleic acid encoding an RNA guided nuclease-null DNA binding protein of a Type II CRISPR System that binds to the DNA and is guided by the one or more RNAs, introducing into the cell a third foreign nucleic acid encoding a transcriptional regulator protein or domain, wherein the one or more RNAs, the RNA guided nuclease-null DNA binding protein of a Type II CRISPR System, and the transcriptional regulator protein or domain are expressed, wherein the one or more RNAs, the RNA guided nuclease-null DNA binding protein of a Type II CRISPR System and the transcriptional regulator protein or domain co-localize to the DNA and wherein the transcriptional regulator protein or domain regulates expression of the target nucleic acid.

According to one aspect, the foreign nucleic acid encoding an RNA guided nuclease-null DNA binding protein of a Type II CRISPR System further encodes the transcriptional regulator protein or domain fused to the RNA guided nuclease-null DNA binding protein of a Type II CRISPR System. According to one aspect, the foreign nucleic acid encoding one or more RNAs further encodes a target of an RNA-binding domain and the foreign nucleic acid encoding the transcriptional regulator protein or domain further encodes an RNA-binding domain fused to the transcriptional regulator protein or domain.

According to one aspect, the cell is a eukaryotic cell. According to one aspect, the cell is a yeast cell, a plant cell or an animal cell. According to one aspect, the cell is a mammalian cell.

According to one aspect, the RNA is between about 10 to about 500 nucleotides. According to one aspect, the RNA is between about 20 to about 100 nucleotides.

According to one aspect, the transcriptional regulator protein or domain is a transcriptional activator. According to one aspect, the transcriptional regulator protein or domain upregulates expression of the target nucleic acid. According to one aspect, the transcriptional regulator protein or domain upregulates expression of the target nucleic acid to treat a disease or detrimental condition. According to one aspect, the target nucleic acid is associated with a disease or detrimental condition.

According to one aspect, the one or more RNAs is a guide RNA. According to one aspect, the one or more RNAs is a tracrRNA-crRNA fusion.

According to one aspect, the DNA is genomic DNA, mitochondrial DNA, viral DNA, or exogenous DNA.

According to certain aspects, a method of modulating expression of a target nucleic acid in a cell is provided including introducing into the cell a first foreign nucleic acid encoding one or more RNAs (ribonucleic acids) complementary to DNA (deoxyribonucleic acid), wherein the DNA includes the target nucleic acid, introducing into the cell a second foreign nucleic acid encoding a nuclease-null Cas9 protein that binds to the DNA and is guided by the one or more RNAs, introducing into the cell a third foreign nucleic acid encoding a transcriptional regulator protein or domain, wherein the one or more RNAs, the nuclease-null Cas9 protein, and the transcriptional regulator protein or domain are expressed, wherein the one or more RNAs, the nuclease-null Cas9 protein and the transcriptional regulator protein or domain co-localize to the DNA and wherein the transcriptional regulator protein or domain regulates expression of the target nucleic acid.

According to one aspect, the foreign nucleic acid encoding a nuclease-null Cas9 protein further encodes the transcriptional regulator protein or domain fused to the nuclease-null Cas9 protein. According to one aspect, the foreign nucleic acid encoding one or more RNAs further encodes a target of an RNA-binding domain and the foreign nucleic acid encoding the transcriptional regulator protein or domain further encodes an RNA-binding domain fused to the transcriptional regulator protein or domain.

According to one aspect, the cell is a eukaryotic cell. According to one aspect, the cell is a yeast cell, a plant cell or an animal cell. According to one aspect, the cell is a mammalian cell.

According to one aspect, the RNA is between about 10 to about 500 nucleotides. According to one aspect, the RNA is between about 20 to about 100 nucleotides.

According to one aspect, the transcriptional regulator protein or domain is a transcriptional activator. According to one aspect, the transcriptional regulator protein or domain upregulates expression of the target nucleic acid. According to one aspect, the transcriptional regulator protein or domain upregulates expression of the target nucleic acid to treat a disease or detrimental condition. According to one aspect, the target nucleic acid is associated with a disease or detrimental condition.

According to one aspect, the one or more RNAs is a guide RNA. According to one aspect, the one or more RNAs is a tracrRNA-crRNA fusion.

According to one aspect, the DNA is genomic DNA, mitochondrial DNA, viral DNA, or exogenous DNA.

According to one aspect a cell is provided that includes a first foreign nucleic acid encoding one or more RNAs complementary to DNA, wherein the DNA includes a target nucleic acid, a second foreign nucleic acid encoding an RNA guided nuclease-null DNA binding protein, and a third foreign nucleic acid encoding a transcriptional regulator protein or domain wherein the one or more RNAs, the RNA guided nuclease-null DNA binding protein and the transcriptional regulator protein or domain are members of a co-localization complex for the target nucleic acid.

According to one aspect, the foreign nucleic acid encoding an RNA guided nuclease-null DNA binding protein further encodes the transcriptional regulator protein or domain fused to an RNA guided nuclease-null DNA binding protein. According to one aspect, the foreign nucleic acid encoding one or more RNAs further encodes a target of an RNA-binding domain and the foreign nucleic acid encoding the transcriptional regulator protein or domain further encodes an RNA-binding domain fused to the transcriptional regulator protein or domain.

According to one aspect, the cell is a eukaryotic cell. According to one aspect, the cell is a yeast cell, a plant cell or an animal cell. According to one aspect, the cell is a mammalian cell.

According to one aspect, the RNA is between about 10 to about 500 nucleotides. According to one aspect, the RNA is between about 20 to about 100 nucleotides.

According to one aspect, the transcriptional regulator protein or domain is a transcriptional activator. According to one aspect, the transcriptional regulator protein or domain upregulates expression of the target nucleic acid. According to one aspect, the transcriptional regulator protein or domain upregulates expression of the target nucleic acid to treat a disease or detrimental condition. According to one aspect, the target nucleic acid is associated with a disease or detrimental condition.

According to one aspect, the one or more RNAs is a guide RNA. According to one aspect, the one or more RNAs is a tracrRNA-crRNA fusion.

According to one aspect, the DNA is genomic DNA, mitochondrial DNA, viral DNA, or exogenous DNA.

According to certain aspects, the RNA guided nuclease-null DNA binding protein is an RNA guided nuclease-null DNA binding protein of a Type II CRISPR System. According to certain aspects, the RNA guided nuclease-null DNA binding protein is a nuclease-null Cas9 protein.

According to one aspect, a method of altering a DNA target nucleic acid in a cell is provided that includes introducing into the cell a first foreign nucleic acid encoding two or more RNAs with each RNA being complementary to an adjacent site in the DNA target nucleic acid, introducing into the cell a second foreign nucleic acid encoding at least one RNA guided DNA binding protein nickase and being guided by the two or more RNAs, wherein the two or more RNAs and the at least one RNA guided DNA binding protein nickase are expressed and wherein the at least one RNA guided DNA binding protein nickase co-localizes with the two or more RNAs to the DNA target nucleic acid and nicks the DNA target nucleic acid resulting in two or more adjacent nicks.

According to one aspect, a method of altering a DNA target nucleic acid in a cell is provided that includes introducing into the cell a first foreign nucleic acid encoding two or more RNAs with each RNA being complementary to an adjacent site in the DNA target nucleic acid, introducing into the cell a second foreign nucleic acid encoding at least one RNA guided DNA binding protein nickase of a Type II CRISPR System and being guided by the two or more RNAs, wherein the two or more RNAs and the at least one RNA guided DNA binding protein nickase of a Type II CRISPR System are expressed and wherein the at least one RNA guided DNA binding protein nickase of a Type II CRISPR System co-localizes with the two or more RNAs to the DNA target nucleic acid and nicks the DNA target nucleic acid resulting in two or more adjacent nicks.

According to one aspect, a method of altering a DNA target nucleic acid in a cell is provided that includes introducing into the cell a first foreign nucleic acid encoding two or more RNAs with each RNA being complementary to an adjacent site in the DNA target nucleic acid, introducing into the cell a second foreign nucleic acid encoding at least one Cas9 protein nickase having one inactive nuclease domain and being guided by the two or more RNAs, wherein the two or more RNAs and the at least one Cas9 protein nickase are expressed and wherein the at least one Cas9 protein nickase co-localizes with the two or more RNAs to the DNA target nucleic acid and nicks the DNA target nucleic acid resulting in two or more adjacent nicks.

According to the methods of altering a DNA target nucleic acid, the two or more adjacent nicks are on the same strand of the double stranded DNA. According to one aspect, the two or more adjacent nicks are on the same strand of the double stranded DNA and result in homologous recombination. According to one aspect, the two or more adjacent nicks are on different strands of the double stranded DNA. According to one aspect, the two or more adjacent nicks are on different strands of the double stranded DNA and create double stranded breaks. According to one aspect, the two or more adjacent nicks are on different strands of the double stranded DNA and create double stranded breaks resulting in nonhomologous end joining. According to one aspect, the two or more adjacent nicks are on different strands of the double stranded DNA and are offset with respect to one another. According to one aspect, the two or more adjacent nicks are on different strands of the double stranded DNA and are offset with respect to one another and create double stranded breaks. According to one aspect, the two or more adjacent nicks are on different strands of the double stranded DNA and are offset with respect to one another and create double stranded breaks resulting in nonhomologous end joining. According to one aspect, the method further includes introducing into the cell a third foreign nucleic acid encoding a donor nucleic acid sequence wherein the two or more nicks results in homologous recombination of the target nucleic acid with the donor nucleic acid sequence.

According to one aspect, a method of altering a DNA target nucleic acid in a cell is provided including introducing into the cell a first foreign nucleic acid encoding two or more RNAs with each RNA being complementary to an adjacent site in the DNA target nucleic acid, introducing into the cell a second foreign nucleic acid encoding at least one RNA guided DNA binding protein nickase and being guided by the two or more RNAs, and wherein the two or more RNAs and the at least one RNA guided DNA binding protein nickase are expressed and wherein the at least one RNA guided DNA binding protein nickase co-localizes with the two or more RNAs to the DNA target nucleic acid and nicks the DNA target nucleic acid resulting in two or more adjacent nicks, and wherein the two or more adjacent nicks are on different strands of the double stranded DNA and create double stranded breaks resulting in fragmentation of the target nucleic acid thereby preventing expression of the target nucleic acid.

According to one aspect, a method of altering a DNA target nucleic acid in a cell is provided including introducing into the cell a first foreign nucleic acid encoding two or more RNAs with each RNA being complementary to an adjacent site in the DNA target nucleic acid, introducing into the cell a second foreign nucleic acid encoding at least one RNA guided DNA binding protein nickase of a Type II CRISPR system and being guided by the two or more RNAs, and wherein the two or more RNAs and the at least one RNA guided DNA binding protein nickase of a Type II CRISPR System are expressed and wherein the at least one RNA guided DNA binding protein nickase of a Type II CRISPR System co-localizes with the two or more RNAs to the DNA target nucleic acid and nicks the DNA target nucleic acid resulting in two or more adjacent nicks, and wherein the two or more adjacent nicks are on different strands of the double stranded DNA and create double stranded breaks resulting in fragmentation of the target nucleic acid thereby preventing expression of the target nucleic acid.

According to one aspect, a method of altering a DNA target nucleic acid in a cell is provided including introducing into the cell a first foreign nucleic acid encoding two or more RNAs with each RNA being complementary to an adjacent site in the DNA target nucleic acid, introducing into the cell a second foreign nucleic acid encoding at least one Cas9 protein nickase having one inactive nuclease domain and being guided by the two or more RNAs, and wherein the two or more RNAs and the at least one Cas9 protein nickase are expressed and wherein the at least one Cas9 protein nickase co-localizes with the two or more RNAs to the DNA target nucleic acid and nicks the DNA target nucleic acid resulting in two or more adjacent nicks, and wherein the two or more adjacent nicks are on different strands of the double stranded DNA and create double stranded breaks resulting in fragmentation of the target nucleic acid thereby preventing expression of the target nucleic acid.

According to one aspect, a cell is provided including a first foreign nucleic acid encoding two or more RNAs with each RNA being complementary to an adjacent site in a DNA target nucleic acid, and a second foreign nucleic acid encoding at least one RNA guided DNA binding protein nickase, and wherein the two or more RNAs and the at least one RNA guided DNA binding protein nickase are members of a co-localization complex for the DNA target nucleic acid.

According to one aspect, the RNA guided DNA binding protein nickase is an RNA guided DNA binding protein nickase of a Type II CRISPR System. According to one aspect, the RNA guided DNA binding protein nickase is a Cas9 protein nickase having one inactive nuclease domain.

According to one aspect, the cell is a eukaryotic cell. According to one aspect, the cell is a yeast cell, a plant cell or an animal cell. According to one aspect, the cell is a mammalian cell.

According to one aspect, the RNA includes between about 10 to about 500 nucleotides. According to one aspect, the RNA includes between about 20 to about 100 nucleotides.

According to one aspect, the target nucleic acid is associated with a disease or detrimental condition.

According to one aspect, the two or more RNAs are guide RNAs. According to one aspect, the two or more RNAs are tracrRNA-crRNA fusions.

According to one aspect, the DNA target nucleic acid is genomic DNA, mitochondrial DNA, viral DNA, or exogenous DNA.

Further features and advantages of certain embodiments of the present invention will become more fully apparent in the following description of embodiments and drawings thereof, and from the claims.

### BRIEF DESCRIPTION OF THE DRAWINGS

The patent or application file contains drawings executed in color. Copies of this patent or patent application publication with the color drawings will be provided by the Office upon request and payment of the necessary fee. The foregoing and other features and advantages of the present embodiments will be more fully understood from the following detailed description of illustrative embodiments taken in conjunction with the accompanying drawings in which:

FIG. 1A and FIG. 1B are schematics of RNA-guided transcriptional activation. FIG. 1C is a design of a reporter construct. FIGS. 1D-1 and 1D-2 show data demonstrating that Cas9N-VP64 fusions display RNA-guided transcriptional activation as assayed by both fluorescence-activated cell sorting (FACS) and immunofluorescence assays (IF). FIGS. 1E-1 and 1E-2 show assay data by FACS and IF demonstrating gRNA sequence-specific transcriptional activation from reporter constructs in the presence of Cas9N, MS2-VP64 and gRNA bearing the appropriate MS2 aptamer binding sites. FIG. 1F depicts data demonstrating transcriptional induction by individual gRNAs and multiple gRNAs.

FIG. 2A depicts a methodology for evaluating the landscape of targeting by Cas9-gRNA complexes and TALEs. FIG. 2B depicts data demonstrating that a Cas9-gRNA complex is on average tolerant to 1-3 mutations in its target sequences. FIG. 2C depicts data demonstrating that the Cas9-gRNA complex is largely insensitive to point mutations, except those localized to the PAM sequence. FIG. 2D depicts heat plot data demonstrating that introduction of 2 base mismatches significantly impairs the Cas9-gRNA complex activity. FIG. 2E depicts data demonstrating that an 18-mer TALE reveals is on average tolerant to 1-2 mutations in its target sequence. FIG. 2F depicts data demonstrating the 18-mer TALE is, similar to the Cas9-gRNA complexes, largely insensitive to single base mismatched in its target. FIG. 2G depicts heat plot data demonstrating that introduction of 2 base mismatches significantly impairs the 18-mer TALE activity.

FIG. 3A depicts a schematic of a guide RNA design. FIG. 3B depicts data showing percentage rate of non-homologous end joining for off-set nicks leading to 5′ overhangs and off-set nicks leading to 3′ overhangs. FIG. 3C depicts data showing percentage rate of targeting for off-set nicks leading to 5′ overhangs and off-set nicks leading to 3′ overhangs.

FIG. 4A is a schematic of a metal coordinating residue in RuvC PDB ID: 4EP4 (blue) position D7 (left), a schematic of HNH endonuclease domains from PDB IDs: 3M7K (orange) and 4H9D (cyan) including a coordinated Mg-ion (gray sphere) and DNA from 3M7K (purple) (middle) and a list of mutants analyzed (right). FIG. 4B depicts data showing undetectable nuclease activity for Cas9 mutants m3 and m4, and also their respective fusions with VP64. FIG. 4C is a higher-resolution examination of the data in FIG. 4B.

FIG. 5A is a schematic of a homologous recombination assay to determine Cas9-gRNA activity. FIGS. 5B-1 and 5B-2 depict guide RNAs with random sequence insertions and percentage rate of homologous recombination.

FIG. 6A is a schematic of guide RNAs for the OCT4 gene. FIG. 6B depicts transcriptional activation for a promoter-luciferase reporter construct. FIG. 6C depicts transcriptional activation via qPCR of endogenous genes.

FIG. 7A is a schematic of guide RNAs for the REX1 gene. FIG. 7B depicts transcriptional activation for a promoter-luciferase reporter construct. FIG. 7C depicts transcriptional activation via qPCR of endogenous genes.

FIG. 8A depicts in schematic a high level specificity analysis processing flow for calculation of normalized expression levels. FIG. 8B depicts data of distributions of percentages of binding sites by numbers of mismatches generated within a biased construct library. Left: Theoretical distribution. Right: Distribution observed from an actual TALE construct library. FIG. 8C depicts data of distributions of percentages of tag counts aggregated to binding sites by numbers of mismatches. Left: Distribution observed from the positive control sample. Right: Distribution observed from a sample in which a non-control TALE was induced.

FIG. 9A depicts data for analysis of the targeting landscape of a Cas9-gRNA complex showing tolerance to 1-3 mutations in its target sequence. FIG. 9B depicts data for analysis of the targeting landscape of a Cas9-gRNA complex showing insensitivity to point mutations, except those localized to the PAM sequence. FIG. 9C depicts heat plot data for analysis of the targeting landscape of a Cas9-gRNA complex showing that introduction of 2 base mismatches significantly impairs activity. FIG. 9D depicts data from a nuclease mediated HR assay confirming that the predicted PAM for the S. pyogenes Cas9 is NGG and also NAG.

FIGS. 10A-1 and 10A-2 depict data from a nuclease mediated HR assay confirming that 18-mer TALEs tolerate multiple mutations in their target sequences. FIG. 10B depicts data from analysis of the targeting landscape of TALEs of 3 different sizes (18-mer, 14-mer and 10-mer). FIG. 10C depicts data for 10-mer TALEs show near single-base mismatch resolution. FIG. 10D depicts heat plot data for 10-mer TALEs show near single-base mismatch resolution.

FIG. 11A depicts designed guide RNAs. FIG. 11B depicts percentage rate of non-homologous end joining for various guide RNAs.

FIG. 12A depicts the Sox2 gene. FIG. 12B depicts the Nanog gene.

FIGS. 13A-13F depict the targeting landscape of two additional Cas9-gRNA complexes.

FIG. 14A depicts the specificity profile of two gRNAs (wild-type) and mutants. Sequence differences are highlighted in red. FIGS. 14B and 14C depict that this assay was specific for the gRNA being evaluated (data re-plotted from FIG. 13D).

FIGS. 15A, 15B-1, 15B-2, 15C, 15D-1, and 15D-2 depict gRNA2 (FIGS. 15A-B) and gRNA3 (FIGS. 15C-D) bearing single or double-base mismatches (highlighted in red) in the spacer sequence versus the target.

FIGS. 16A, 16B-1, 16B-2, 16C, 16D-1, and 16D-2 depict a nuclease assay of two independent gRNA that were tested: gRNA1 (FIGS. 16A-B) and gRNA3 (FIGS. 16C-D) bearing truncations at the 5′ end of their spacer.

FIGS. 17A-17B depict a nuclease mediated HR assay that shows the PAM for the S. pyogenes Cas9 is NGG and also NAG.

FIGS. 18A-18B depict a nuclease mediated HR assay that confirmed that 18-mer TALEs tolerate multiple mutations in their target sequences.

FIGS. 19A, 19B-1, 19B-2, 19C-1, and 19C-2 depict a comparison of TALE monomer specificity versus TALE protein specificity. FIGS. 20A-20B depict data related to off-set nicking.

FIGS. 21A-21C depict off-set nicking and NHEJ profiles.

### DETAILED DESCRIPTION

Embodiments of the present disclosure are based on the use of DNA binding proteins to co-localize transcriptional regulator proteins or domains to DNA in a manner to regulate a target nucleic acid. Such DNA binding proteins are readily known to those of skill in the art to bind to DNA for various purposes. Such DNA binding proteins may be naturally occurring. DNA binding proteins included within the scope of the present disclosure include those which may be guided by RNA, referred to herein as guide RNA. According to this aspect, the guide RNA and the RNA guided DNA binding protein form a co-localization complex at the DNA. According to certain aspects, the DNA binding protein may be a nuclease-null DNA binding protein. According to this aspect, the nuclease-null DNA binding protein may result from the alteration or modification of a DNA binding protein having nuclease activity. Such DNA binding proteins having nuclease activity are known to those of skill in the art, and include naturally occurring DNA binding proteins having nuclease activity, such as Cas9 proteins present, for example, in Type II CRISPR systems. Such Cas9 proteins and Type II CRISPR systems are well documented in the art. See Makarova et al., Nature Reviews, Microbiology, Vol. 9, June 2011, pp. 467-477 including all supplementary information hereby incorporated by reference in its entirety.

Exemplary DNA binding proteins having nuclease activity function to nick or cut double stranded DNA. Such nuclease activity may result from the DNA binding protein having one or more polypeptide sequences exhibiting nuclease activity. Such exemplary DNA binding proteins may have two separate nuclease domains with each domain responsible for cutting or nicking a particular strand of the double stranded DNA. Exemplary polypeptide sequences having nuclease activity known to those of skill in the art include the MerA-HNH nuclease related domain and the RuvC-like nuclease domain. Accordingly, exemplary DNA binding proteins are those that in nature contain one or more of the MerA-HNH nuclease related domain and the RuvC-like nuclease domain. According to certain aspects, the DNA binding protein is altered or otherwise modified to inactivate the nuclease activity. Such alteration or modification includes altering one or more amino acids to inactivate the nuclease activity or the nuclease domain. Such modification includes removing the polypeptide sequence or polypeptide sequences exhibiting nuclease activity, i.e. the nuclease domain, such that the polypeptide sequence or polypeptide sequences exhibiting nuclease activity, i.e. nuclease domain, are absent from the DNA binding protein. Other modifications to inactivate nuclease activity will be readily apparent to one of skill in the art based on the present disclosure. Accordingly, a nuclease-null DNA binding protein includes polypeptide sequences modified to inactivate nuclease activity or removal of a polypeptide sequence or sequences to inactivate nuclease activity. The nuclease-null DNA binding protein retains the ability to bind to DNA even though the nuclease activity has been inactivated. Accordingly, the DNA binding protein includes the polypeptide sequence or sequences required for DNA binding but may lack the one or more or all of the nuclease sequences exhibiting nuclease activity. Accordingly, the DNA binding protein includes the polypeptide sequence or sequences required for DNA binding but may have one or more or all of the nuclease sequences exhibiting nuclease activity inactivated.

According to one aspect, a DNA binding protein having two or more nuclease domains may be modified or altered to inactivate all but one of the nuclease domains. Such a modified or altered DNA binding protein is referred to as a DNA binding protein nickase, to the extent that the DNA binding protein cuts or nicks only one strand of double stranded DNA. When guided by RNA to DNA, the DNA binding protein nickase is referred to as an RNA guided DNA binding protein nickase.

An exemplary DNA binding protein is an RNA guided DNA binding protein of a Type II CRISPR System which lacks nuclease activity. An exemplary DNA binding protein is a nuclease-null Cas9 protein. An exemplary DNA binding protein is a Cas9 protein nickase.

In S. pyogenes, Cas9 generates a blunt-ended double-stranded break 3bp upstream of the protospacer-adjacent motif (PAM) via a process mediated by two catalytic domains in the protein: an HNH domain that cleaves the complementary strand of the DNA and a RuvC-like domain that cleaves the non-complementary strand. See Jinke et al., Science 337, 816-821 (2012) hereby incorporated by reference in its entirety. Cas9 proteins are known to exist in many Type II CRISPR systems including the following as identified in the supplementary information to Makarova et al., Nature Reviews, Microbiology. Vol. 9. June 2011. pp. 467-477: Methanococcus maripaludis C7; Methanococcus maripaludis ; Corynebacterium efficiens YS-314; Corynebacterium glutamicum ATCC 13032 Kitasato; Corynebacterium glutamicum ATCC 13032 Bielefeld; Corynebacterium glutamicum R; Corynebacterium kroppenstedtii DSM 44385; Mycobacterium abscessus ATCC 19977; Nocardia farcinica IFM10152; Rhodococcus erythropolis PR4; Rhodococcus jostii RHA1; Rhodococcus opacus B4 uid36573; Acidothermus cellulolyticus 11B; Arthrobacter chlorophenolicus A6; Kribbella flavida DSM 17836 uid43465; Thermomonospora curvata DSM 43183; Bifidobacterium dentium Bd1; Bifidobacterium longum DJO10A; Slackia heliotrinireducens DSM 20476; Persephonella marina EX H1; Bacteroides fragilis NCTC 9434; Capnocytophaga ochracca DSM 7271; Flavobacterium psychrophilum JIP02 86; Akkermansia muciniphila ATCC BAA 835; Rosciflexus castenholzii DSM 13941; Rosciflexus RS1; Syncchocystis PCC6803; Elusimicrobium minutum Pei191; uncultured Termite group 1 bacterium phylotype Rs D17; Fibrobacter succinogenes S85; Bacillus cereus ATCC 10987; Listeria innocua;Lactobacillus casci; Lactobacillus rhamnosus GG; Lactobacillus salivarius UCC118; Streptococcus agalactiae A909; Streptococcus agalactiae NEM316; Streptococcus agalactiae 2603; Streptococcus dysgalactiac equisimilis GGS 124; Streptococcus equi zooepidemicus MGCS10565; Streptococcus gallolyticus UCN34 uid46061; Streptococcus gordonii Challis subst CH1; Streptococcus mutans NN2025 uid46353; Streptococcus mutans; Streptococcus pyogenes M1 GAS; Streptococcus pyogenes MGAS5005; Streptococcus pyogenes MGAS2096; Streptococcus pyogenes MGAS9429; Streptococcus pyogenes MGAS10270; Streptococcus pyogenes MGAS6180; Streptococcus pyogenes MGAS315; Streptococcus pyogenes SSI-1; Streptococcus pyogenes MGAS10750; Streptococcus pyogenes NZ131; Streptococcus thermophiles CNRZ1066; Streptococcus thermophiles LMD-9; Streptococcus thermophiles LMG 18311; Clostridium botulinum A3 Loch Marce; Clostridium botulinum B Eklund 17B; Clostridium botulinum Ba4 657; Clostridium botulinum F Langeland; Clostridium cellulolyticum H10; Finegoldia magna ATCC 29328; Eubacterium rectale ATCC 33656; Mycoplasma gallisepticum; Mycoplasma mobile 163K; Mycoplasma penctrans; Mycoplasma synoviac 53; Streptobacillus moniliformis DSM 12112; Bradyrhizobium BTAil; Nitrobacter hamburgensis X14; Rhodopseudomonas palustris BisB18; Rhodopseudomonas palustris BisB5; Parvibaculum lavamentivorans DS-1; Dinoroscobacter shibac DFL 12; Gluconacetobacter diazotrophicus Pal 5 FAPERJ; Gluconacetobacter diazotrophicus Pal 5 JGI; Azospirillum B510 uid46085; Rhodospirillum rubrum ATCC 11170; Diaphorobacter TPSY uid29975; Verminephrobacter eiseniae EF01-2; Neisseria meningitides 053442; Neisseria meningitides alpha14; Neisseria meningitides Z2491; Desulfovibrio salexigens DSM 2638; Campylobacter jejuni doylei 269 97; Campylobacter jejuni 81116; Campylobacter jejuni; Campylobacter lari RM2100; Helicobacter hepaticus; Wolinella succinogenes; Tolumonas auensis DSM 9187; Pseudoalteromonas atlantica T6c; Shewanella pealeana ATCC 700345; Legionella pneumophila Paris; Actinobacillus succinogenes 130Z; Pasteurella multocida; Francisella tularensis novicida U112; Francisella tularensis holarctica; Francisella tularensis FSC 198; Francisella tularensis tularensis; Francisella tularensis WY96-3418; and Treponema denticola ATCC 35405. Accordingly, aspects of the present disclosure are directed to a Cas9 protein present in a Type II CRISPR system, which has been rendered nuclease null or which has been rendered a nickase as described herein.

The Cas9 protein may be referred by one of skill in the art in the literature as Csn1. The S. pyogenes Cas9 protein sequence that is the subject of experiments described herein is shown below. See Deltcheva et al., Nature 471, 602-607 (2011) hereby incorporated by reference in its entirety.

(SEQ ID NO: 1)
MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD-

According to certain aspects of methods of RNA-guided genome regulation described herein, Cas9 is altered to reduce, substantially reduce or eliminate nuclease activity. According to one aspect, Cas9 nuclease activity is reduced, substantially reduced or eliminated by altering the RuvC nuclease domain or the HNH nuclease domain. According to one aspect, the RuvC nuclease domain is inactivated. According to one aspect, the HNH nuclease domain is inactivated. According to one aspect, the RuvC nuclease domain and the HNH nuclease domain are inactivated. According to an additional aspect, Cas9 proteins are provided where the RuvC nuclease domain and the HNH nuclease domain are inactivated. According to an additional aspect, nuclease-null Cas9 proteins are provided insofar as the RuvC nuclease domain and the HNH nuclease domain are inactivated. According to an additional aspect, a Cas9 nickase is provided where either the RuvC nuclease domain or the HNH nuclease domain is inactivated, thereby leaving the remaining nuclease domain active for nuclease activity. In this manner, only one strand of the double stranded DNA is cut or nicked.

According to an additional aspect, nuclease-null Cas9 proteins are provided where one or more amino acids in Cas9 are altered or otherwise removed to provide nuclease-null Cas9 proteins. According to one aspect, the amino acids include D10 and H840. See Jinke et al., Science 337, 816-821 (2012). According to an additional aspect, the amino acids include D839 and N863. According to one aspect, one or more or all of D10, H840, D839 and H863 are substituted with an amino acid which reduces, substantially eliminates or eliminates nuclease activity. According to one aspect, one or more or all of D10, H840, D839 and H863 are substituted with alanine. According to one aspect, a Cas9 protein having one or more or all of D10, H840, D839 and H863 substituted with an amino acid which reduces, substantially eliminates or eliminates nuclease activity, such as alanine, is referred to as a nuclease-null Cas9 or Cas9N and exhibits reduced or eliminated nuclease activity, or nuclease activity is absent or substantially absent within levels of detection. According to this aspect, nuclease activity for a Cas9N may be undetectable using known assays, i.e. below the level of detection of known assays.

According to one aspect, the nuclease null Cas9 protein includes homologs and orthologs thereof which retain the ability of the protein to bind to the DNA and be guided by the RNA. According to one aspect, the nuclease null Cas9 protein includes the sequence as set forth for naturally occurring Cas9 from S. pyogenes and having one or more or all of D10, H840, D839 and H863 substituted with alanine and protein sequences having at least 30%, 40%, 50%, 60%, 70%, 80%, 90%, 95%, 98% or 99% homology thereto and being a DNA binding protein, such as an RNA guided DNA binding protein.

According to one aspect, the nuclease null Cas9 protein includes the sequence as set forth for naturally occurring Cas9 from S. pyogenes excepting the protein sequence of the RuvC nuclease domain and the HNH nuclease domain and also protein sequences having at least 30%, 40%, 50%, 60%, 70%, 80%, 90%, 95%, 98% or 99% homology thereto and being a DNA binding protein, such as an RNA guided DNA binding protein. In this manner, aspects of the present disclosure include the protein sequence responsible for DNA binding, for example, for co-localizing with guide RNA and binding to DNA and protein sequences homologous thereto, and need not include the protein sequences for the RuvC nuclease domain and the HNH nuclease domain (to the extent not needed for DNA binding), as these domains may be either inactivated or removed from the protein sequence of the naturally occurring Cas9 protein to produce a nuclease null Cas9 protein.

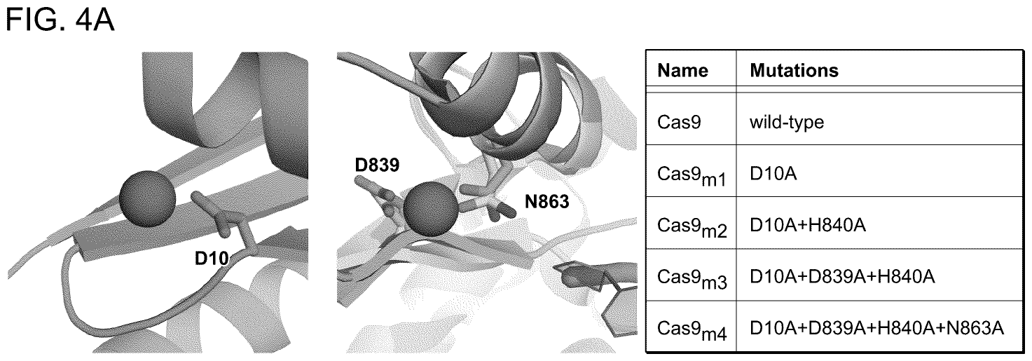

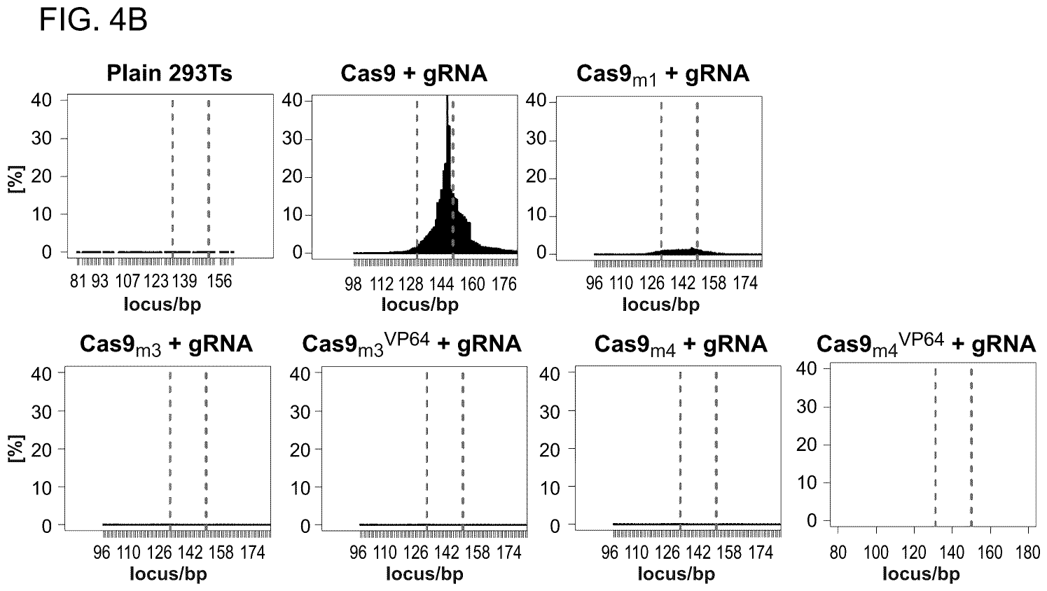

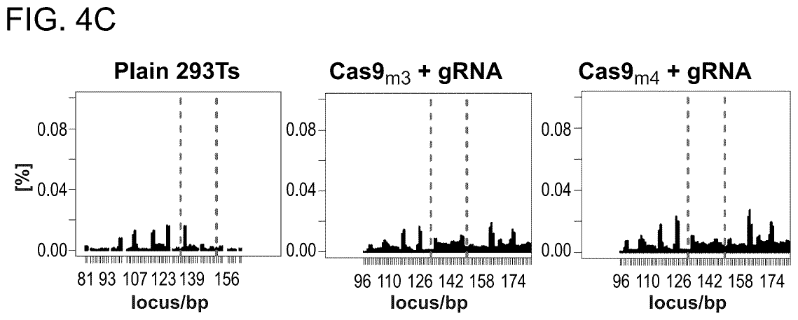

For purposes of the present disclosure, FIG. 4A depicts metal coordinating residues in known protein structures with homology to Cas9. Residues are labeled based on position in Cas9 sequence. Left: RuvC structure, PDB ID: 4EP4 (blue) position D7, which corresponds to D10 in the Cas9 sequence, is highlighted in a Mg-ion coordinating position. Middle: Structures of HNH endonuclease domains from PDB IDs: 3M7K (orange) and 4H9D (cyan) including a coordinated Mg-ion (gray sphere) and DNA from 3M7K (purple). Residues D92 and N113 in 3M7K and 4H9D positions D53 and N77, which have sequence homology to Cas9 amino acids D839 and N863, are shown as sticks. Right: List of mutants made and analyzed for nuclease activity: Cas9 wildtype; Cas9ₘ₁ which substitutes alanine for D10; Cas9ₘ₂ which substitutes alanine for D10 and alanine for H840; Cas9ₘ₃ which substitutes alanine for D10, alanine for H840, and alanine for D839; and Cas9ₘ₄ which substitutes alanine for D10, alanine for H840, alanine for D839, and alanine for N863.

As shown in FIG. 4B, the Cas9 mutants: m3 and m4, and also their respective fusions with VP64 showed undetectable nuclease activity upon deep sequencing at targeted loci. The plots show the mutation frequency versus genomic position, with the red lines demarcating the gRNA target. FIG. 4C is a higher-resolution examination of the data in FIG. 4B and confirms that the mutation landscape shows comparable profile as unmodified loci.

According to one aspect, an engineered Cas9-gRNA system is provided which enables RNA-guided genome regulation in human cells by tethering transcriptional activation domains to either a nuclease-null Cas9 or to guide RNAs. According to one aspect of the present disclosure, one or more transcriptional regulatory proteins or domains (such terms are used interchangeably) are joined or otherwise connected to a nuclease-deficient Cas9 or one or more guide RNA (gRNA). The transcriptional regulatory domains correspond to targeted loci. Accordingly, aspects of the present disclosure include methods and materials for localizing transcriptional regulatory domains to targeted loci by fusing, connecting or joining such domains to either Cas9N or to the gRNA.

According to one aspect, a Cas9N-fusion protein capable of transcriptional activation is provided. According to one aspect, a VP64 activation domain (see Zhang et al., Nature Biotechnology 29, 149-153 (2011) hereby incorporated by reference in its entirety) is joined, fused, connected or otherwise tethered to the C terminus of Cas9N. According to one method, the transcriptional regulatory domain is provided to the site of target genomic DNA by the Cas9N protein. According to one method, a Cas9N fused to a transcriptional regulatory domain is provided within a cell along with one or more guide RNAs. The Cas9N with the transcriptional regulatory domain fused thereto bind at or near target genomic DNA. The one or more guide RNAs bind at or near target genomic DNA. The transcriptional regulatory domain regulates expression of the target gene. According to a specific aspect, a Cas9N-VP64 fusion activated transcription of reporter constructs when combined with gRNAs targeting sequences near the promoter, thereby displaying RNA-guided transcriptional activation.

According to one aspect, a gRNA-fusion protein capable of transcriptional activation is provided. According to one aspect, a VP64 activation domain is joined, fused, connected or otherwise tethered to the gRNA. According to one method, the transcriptional regulatory domain is provided to the site of target genomic DNA by the gRNA. According to one method, a gRNA fused to a transcriptional regulatory domain is provided within a cell along with a Cas9N protein. The Cas9N binds at or near target genomic DNA. The one or more guide RNAs with the transcriptional regulatory protein or domain fused thereto bind at or near target genomic DNA. The transcriptional regulatory domain regulates expression of the target gene. According to a specific aspect, a Cas9N protein and a gRNA fused with a transcriptional regulatory domain activated transcription of reporter constructs, thereby displaying RNA-guided transcriptional activation.

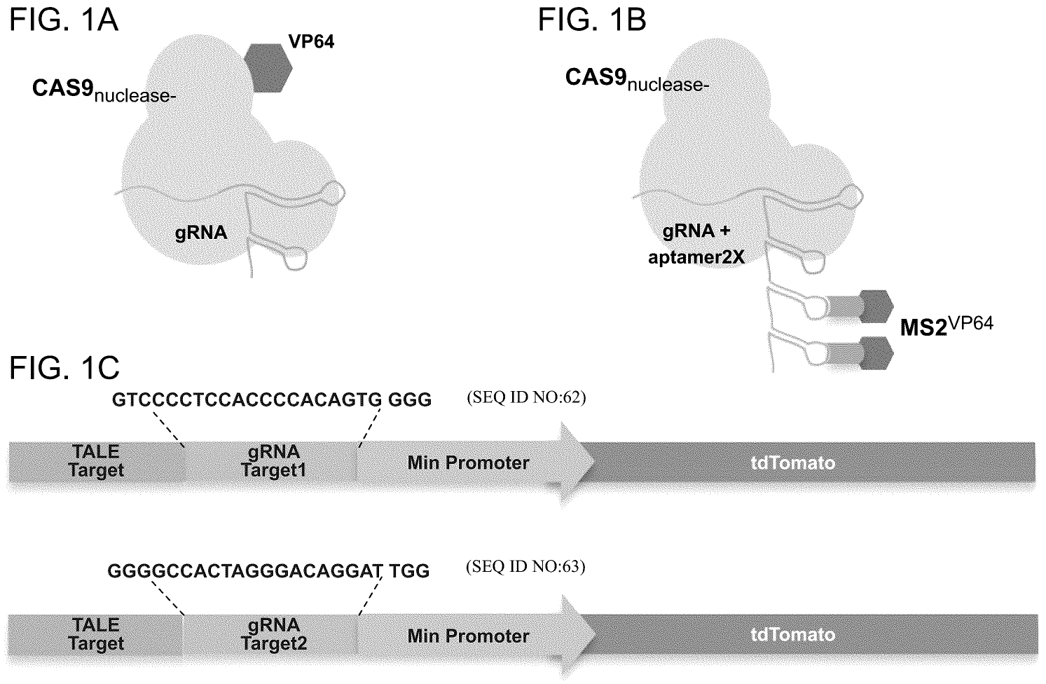

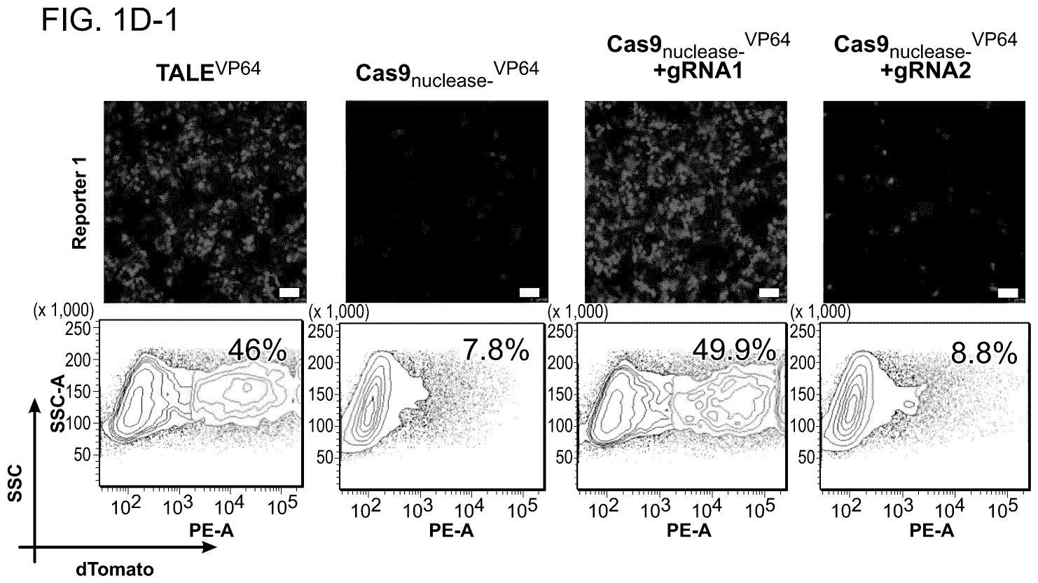

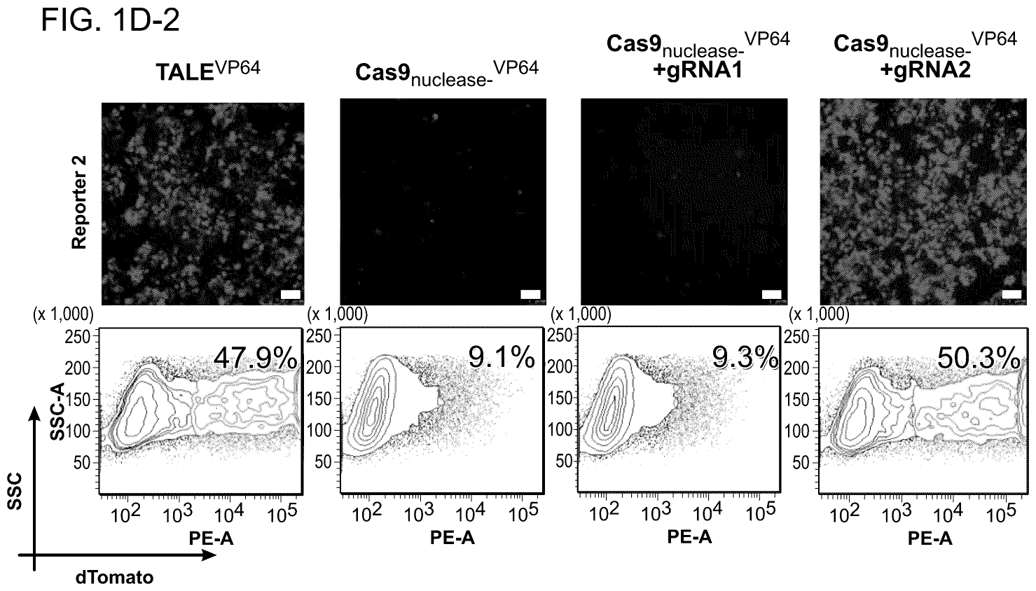

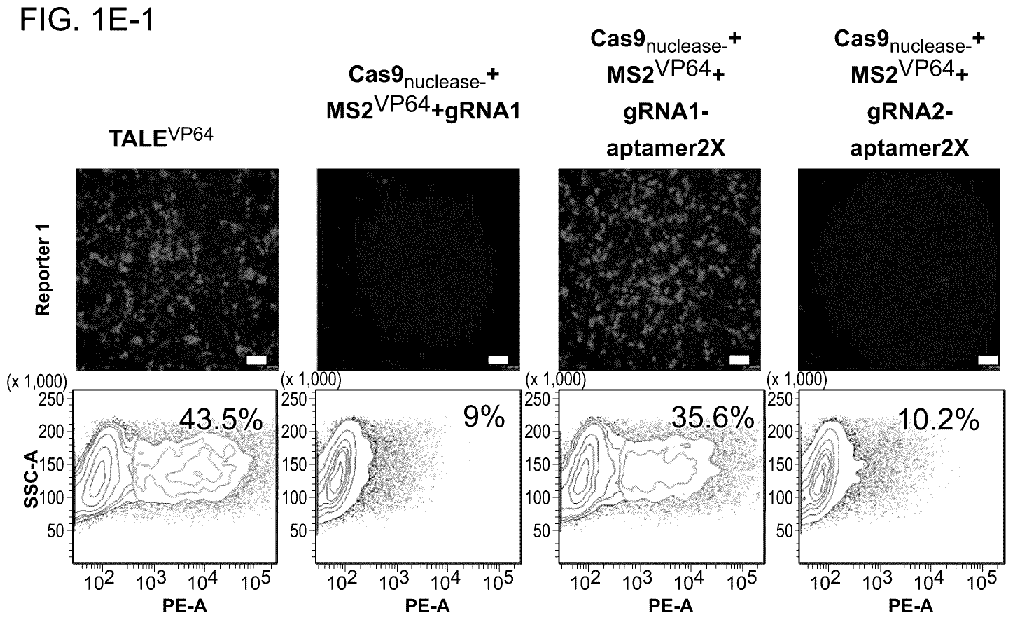

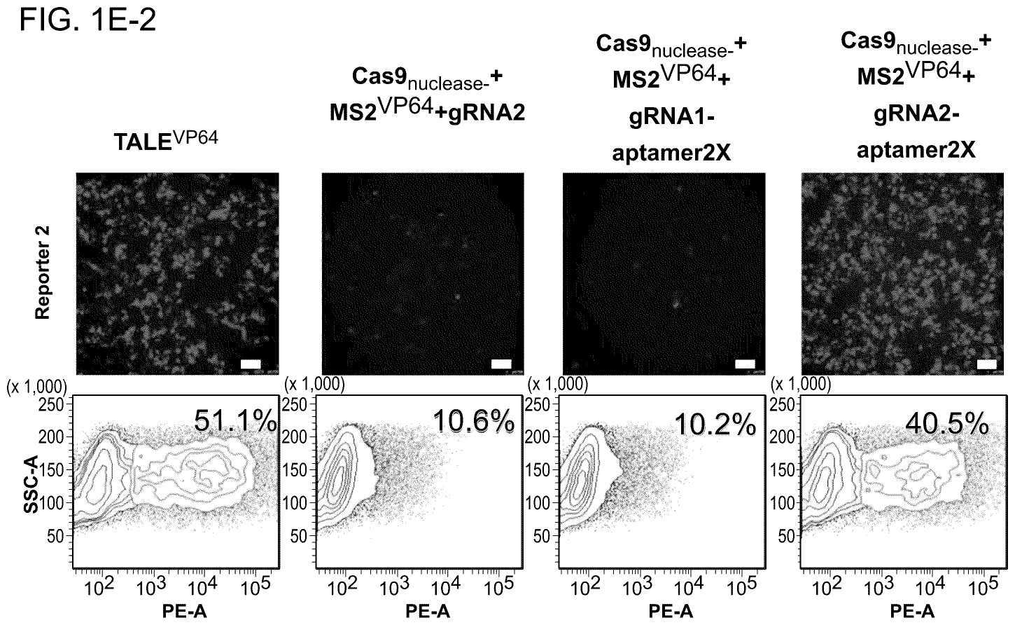

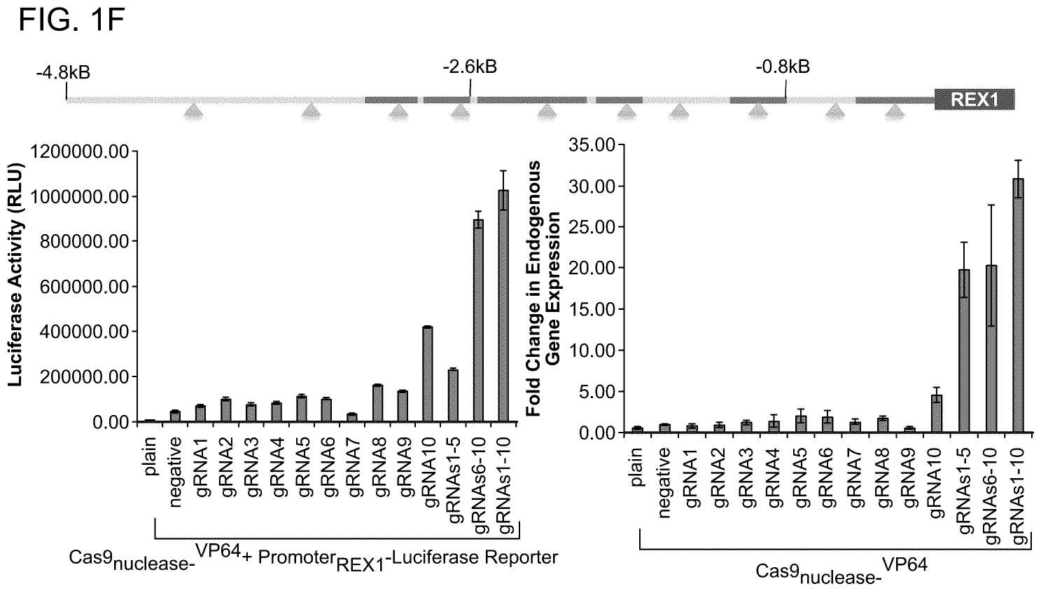

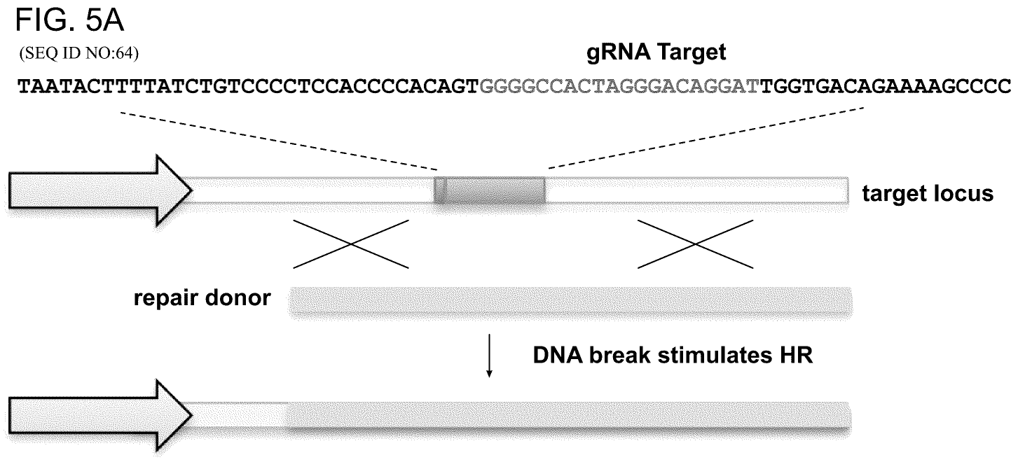

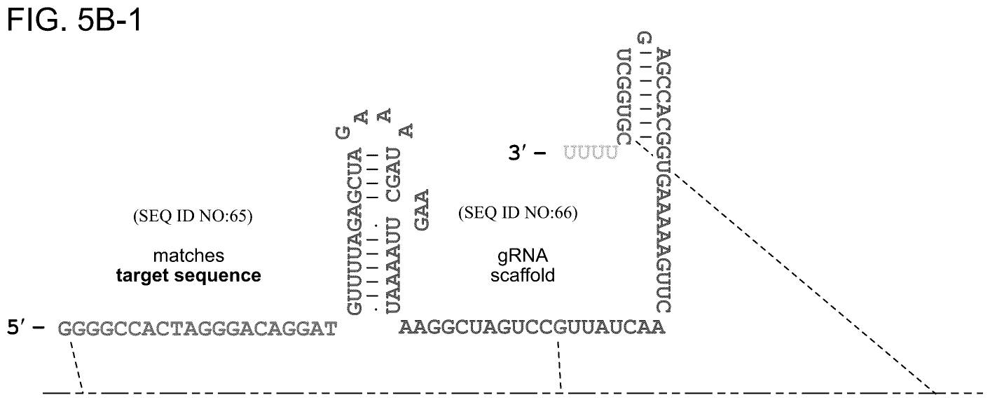

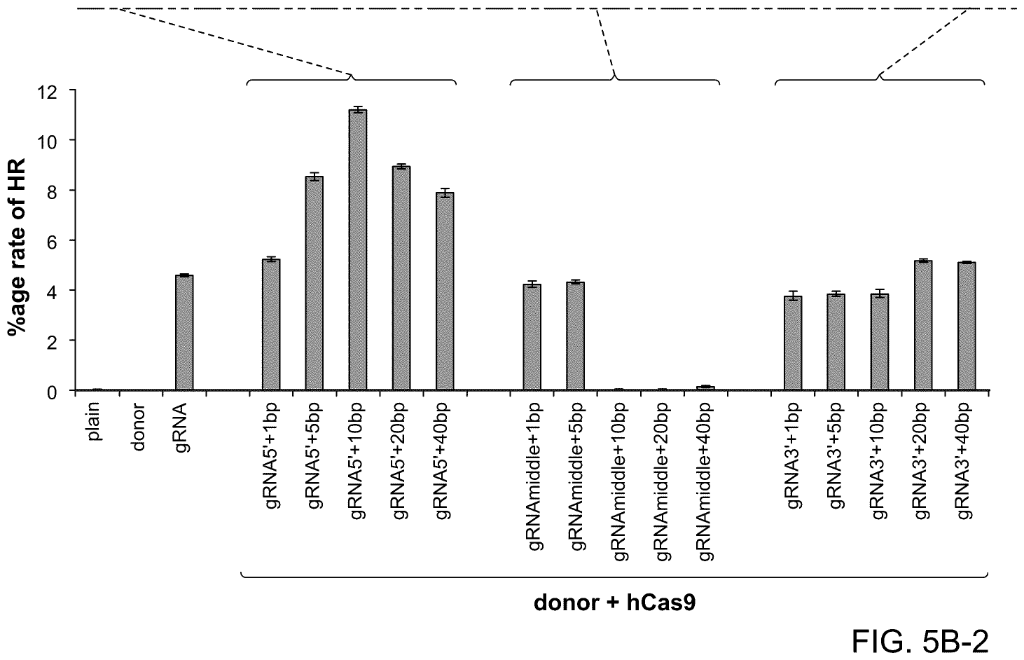

The gRNA tethers capable of transcriptional regulation were constructed by identifying which regions of the gRNA will tolerate modifications by inserting random sequences into the gRNA and assaying for Cas9 function. gRNAs bearing random sequence insertions at either the 5′ end of the crRNA portion or the 3′ end of the tracrRNA portion of a chimeric gRNA retain functionality, while insertions into the tracrRNA scaffold portion of the chimeric gRNA result in loss of function. See FIG. 5A-B summarizing gRNA flexibility to random base insertions. FIG. 5A is a schematic of a homologous recombination (HR) assay to determine Cas9-gRNA activity. As shown in FIGS. 5B-1 and 5B-2. gRNAs bearing random sequence insertions at either the 5′ end of the crRNA portion or the 3′ end of the tracrRNA portion of a chimeric gRNA retain functionality, while insertions into the tracrRNA scaffold portion of the chimeric gRNA result in loss of function. The points of insertion in the gRNA sequence are indicated by red nucleotides. Without wishing to be bound by scientific theory, the increased activity upon random base insertions at the 5′ end may be due to increased half-life of the longer gRNA.

To attach VP64 to the gRNA, two copies of the MS2 bacteriophage coat-protein binding RNA stem-loop were appended to the 3′ end of the gRNA. See Fusco et al., Current Biology: CB13, 161-167 (2003) hereby incorporated by reference in its entirety. These chimeric gRNAs were expressed together with Cas9N and MS2-VP64 fusion protein. Sequence-specific transcriptional activation from reporter constructs was observed in the presence of all 3 components.

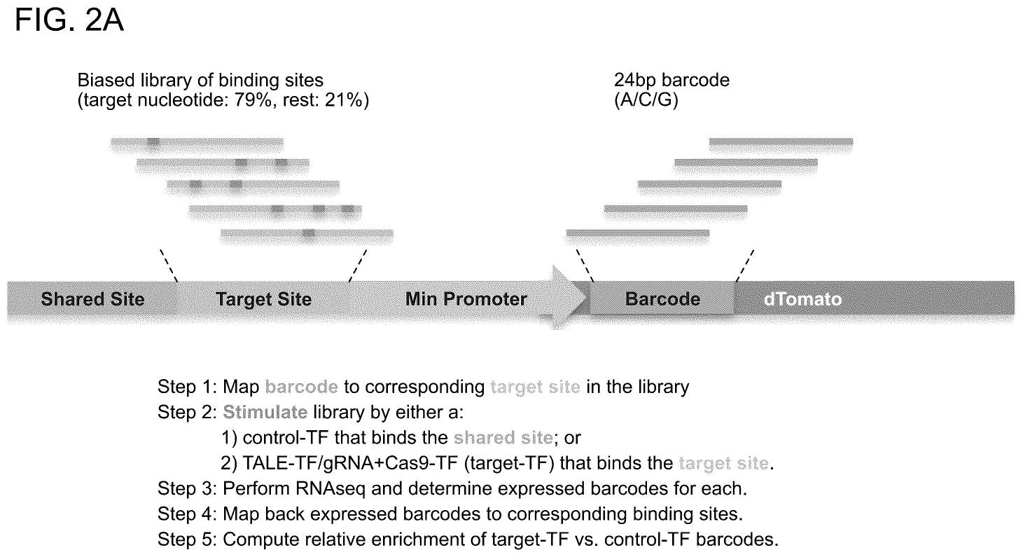

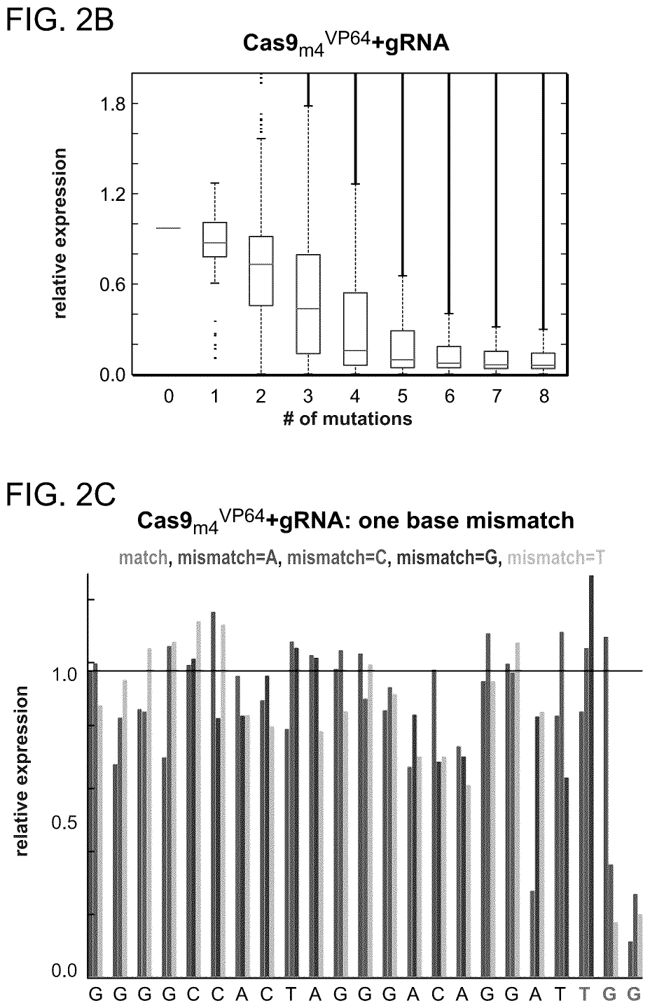

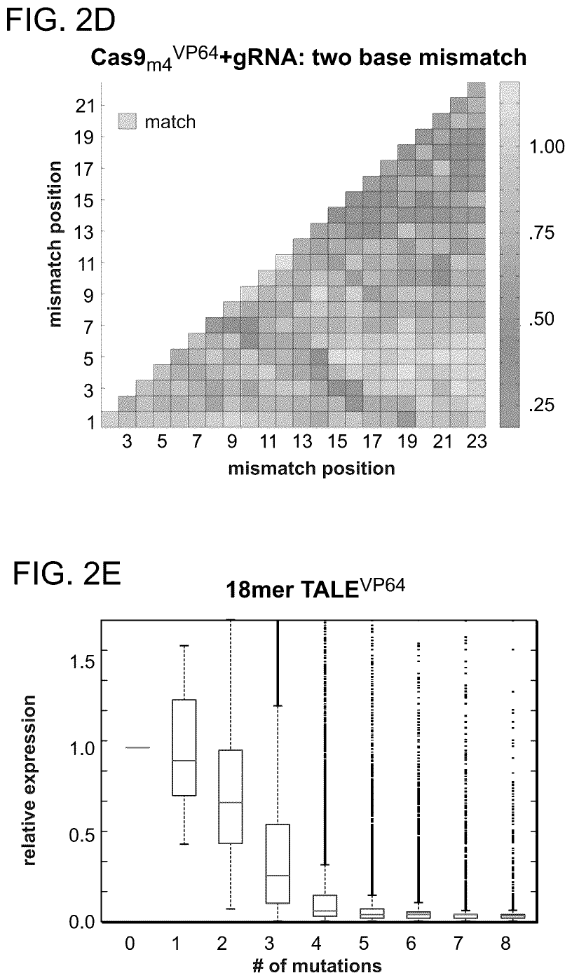

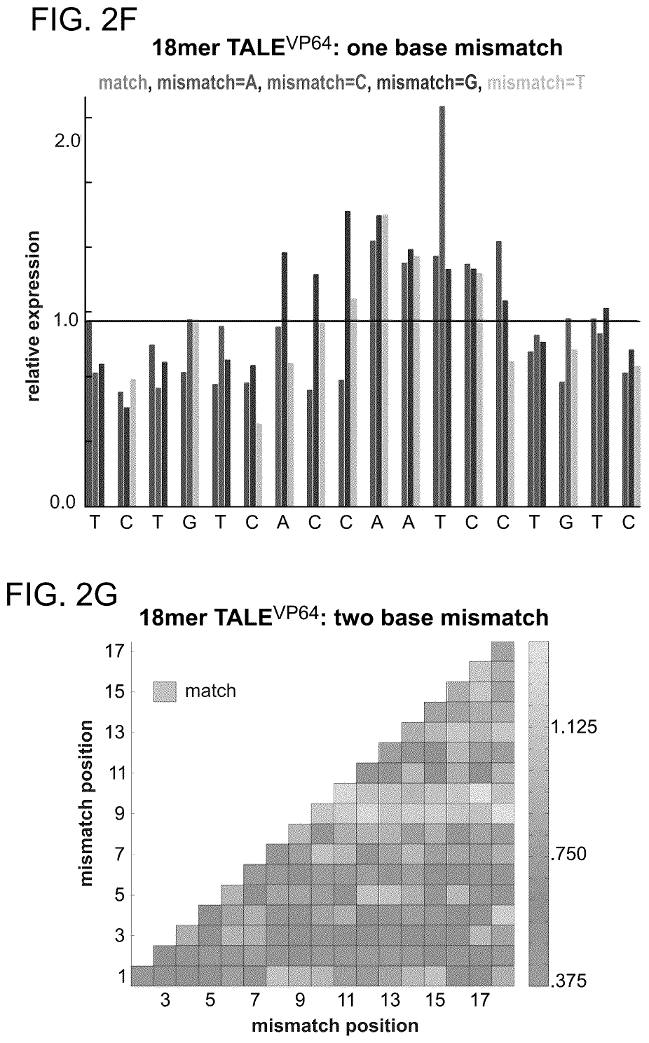

FIG. 1A is a schematic of RNA-guided transcriptional activation. As shown in FIG. 1A, to generate a Cas9N-fusion protein capable of transcriptional activation, the VP64 activation domain was directly tethered to the C terminus of Cas9N. As shown in FIG. 1B, to generate gRNA tethers capable of transcriptional activation, two copies of the MS2 bacteriophage coat-protein binding RNA stem-loop were appended to the 3′ end of the gRNA. These chimeric gRNAs were expressed together with Cas9N and MS2-VP64 fusion protein. FIG. 1C shows design of reporter constructs used to assay transcriptional activation. The two reporters bear distinct gRNA target sites, and share a control TALE-TF target site. As shown in FIGS. 1D-1 and 1D-2, Cas9N-VP64 fusions display RNA-guided transcriptional activation as assayed by both fluorescence-activated cell sorting (FACS) and immunofluorescence assays (IF). Specifically, while the control TALE-TF activated both reporters, the Cas9N-VP64 fusion activates reporters in a gRNA sequence specific manner. As shown in FIGS. 1E-1 and 1E-2, gRNA sequence-specific transcriptional activation from reporter constructs only in the presence of all 3 components: Cas9N, MS2-VP64 and gRNA bearing the appropriate MS2 aptamer binding sites was observed by both FACS and IF.

According to certain aspects, methods are provided for regulating endogenous genes using Cas9N, one or more gRNAs and a transcriptional regulatory protein or domain. According to one aspect, an endogenous gene can be any desired gene, refered to herein as a target gene. According to one exemplary aspect, genes target for regulation included ZFP42 (REX1) and POUSF1 (OCT4), which are both tightly regulated genes involved in maintenance of pluripotency. As shown in FIG. 1F, 10 gRNAs targeting a ˜5kb stretch of DNA upstream of the transcription start site (DNase hypersensitive sites are highlighted in green) were designed for the REX1 gene. Transcriptional activation was assayed using either a promoter-luciferase reporter construct (see Takahashi et al., Cell 131 861-872 (2007) hereby incorporated by reference in its entirety) or directly via qPCR of the endogenous genes.

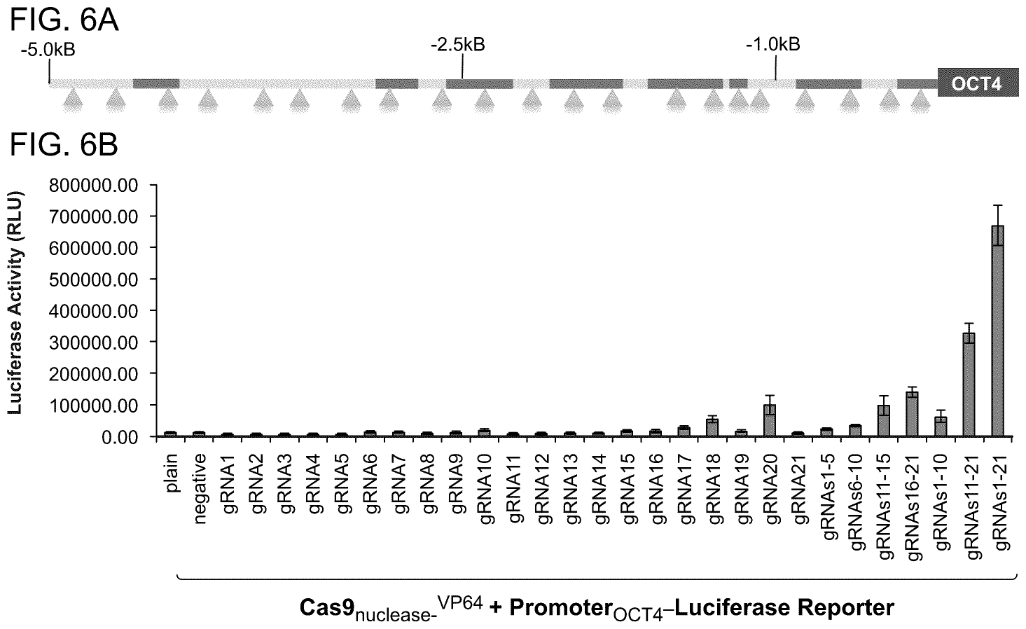

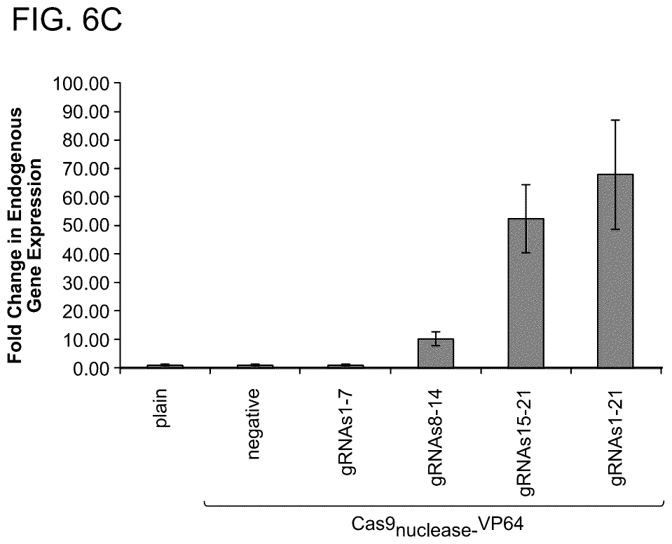

FIGS. 6A-C is directed to RNA-guided OCT4 regulation using Cas9N-VP64. As shown in FIG. 6A. 21 gRNAs targeting a ˜5kb stretch of DNA upstream of the transcription start site were designed for the OCT4 gene. The DNase hypersensitive sites are highlighted in green. FIG. 6B shows transcriptional activation using a promoter-luciferase reporter construct. FIG. 6C shows transcriptional activation directly via qPCR of the endogenous genes. While introduction of individual gRNAs modestly stimulated transcription, multiple gRNAs acted synergistically to stimulate robust multi-fold transcriptional activation.

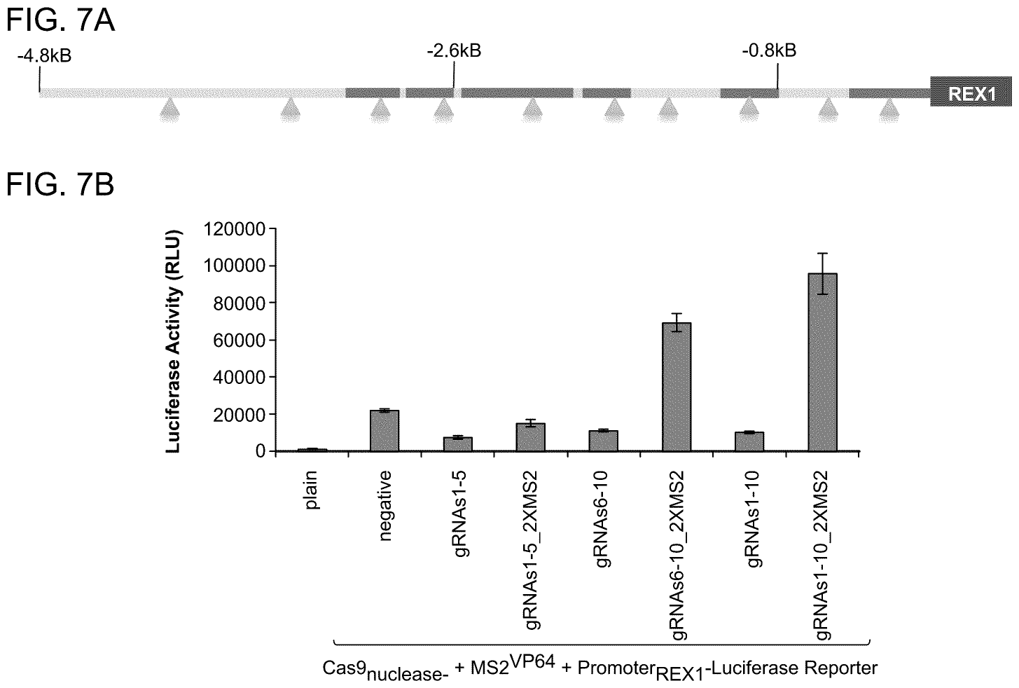

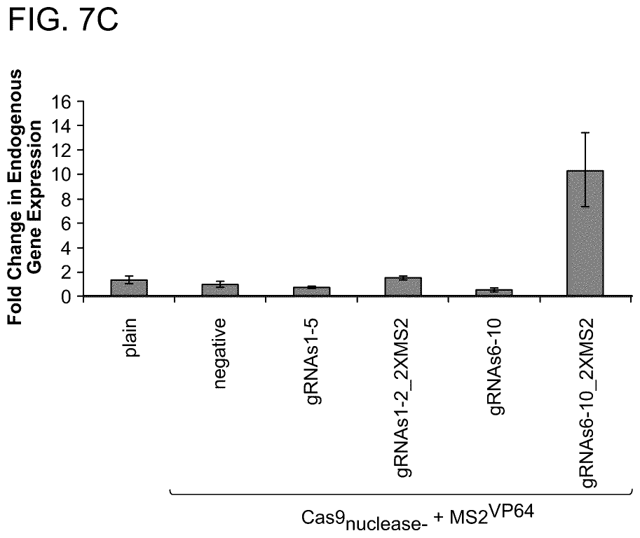

FIGS. 7A-C is directed to RNA-guided REX1 regulation using Cas9N, MS2-VP64 and gRNA+2X-MS2 aptamers. As shown in FIG. 7A. 10 gRNAs targeting a ˜5kb stretch of DNA upstream of the transcription start site were designed for the REX1 gene. The DNase hypersensitive sites are highlighted in green. FIG. 7B shows transcriptional activation using a promoter-luciferase reporter construct. FIG. 7C shows transcriptional activation directly via qPCR of the endogenous genes. While introduction of individual gRNAs modestly stimulated transcription, multiple gRNAs acted synergistically to stimulate robust multi-fold transcriptional activation. In one aspect, the absence of the 2X-MS2 aptamers on the gRNA does not result in transcriptional activation. See Maeder et al., Nature Methods 10, 243-245 (2013) and Perez-Pinera et al., Nature Methods 10, 239-242 (2013) each of which are hereby incorporated by reference in its entirety.

Accordingly, methods are directed to the use of multiple guide RNAs with a Cas9N protein and a transcriptional regulatory protein or domain to regulate expression of a target gene.

Both the Cas9 and gRNA tethering approaches were effective, with the former displaying ˜1.5-2 fold higher potency. This difference is likely due to the requirement for 2-component as opposed to 3-component complex assembly. However, the gRNA tethering approach in principle enables different effector domains to be recruited by distinct gRNAs so long as each gRNA uses a different RNA-protein interaction pair. See Karyer-Bibens et al., Biology of the Cell/Under the Auspices of the European Cell Biology Organization 100. 125-138 (2008) hereby incorporated by reference in its entirety. According to one aspect of the present disclosure, different target genes may be regulated using specific guide RNA and a generic Cas9N protein, i.e. the same or a similar Cas9N protein for different target genes. According to one aspect, methods of multiplex gene regulation are provided using the same or similar Cas9N.

Methods of the present disclosure are also directed to editing target genes using the Cas9N proteins and guide RNAs described herein to provide multiplex genetic and epigenetic engineering of human cells. With Cas9-gRNA targeting being an issue (see Jiang et al., Nature Biotechnology 31, 233-239 (2013) hereby incorporated by reference in its entirety), methods are provided for in-depth interrogation of Cas9 affinity for a very large space of target sequence variations. Accordingly, aspects of the present disclosure provide direct high-throughput readout of Cas9 targeting in human cells, while avoiding complications introduced by dsDNA cut toxicity and mutagenic repair incurred by specificity testing with native nuclease-active Cas9.

Further aspects of the present disclosure are directed to the use of DNA binding proteins or systems in general for the transcriptional regulation of a target gene. One of skill in the art will readily identify exemplary DNA binding systems based on the present disclosure. Such DNA binding systems need not have any nuclease activity, as with the naturally occurring Cas9 protein. Accordingly, such DNA binding systems need not have nuclease activity inactivated. One exemplary DNA binding system is TALE. As a genome editing tool, usually TALE-FokI dimers are used, and for genome regulation TALE-VP64 fusions have been shown to be highly effective. According to one aspect, TALE specificity was evaluated using the methodology shown in FIG. 2A. A construct library in which each element of the library comprises a minimal promoter driving a dTomato fluorescent protein is designed. Downstream of the transcription start site m. a 24bp (A/C/G) random transcript tag is inserted, while two TF binding sites are placed upstream of the promoter: one is a constant DNA sequence shared by all library elements, and the second is a variable feature that bears a ‘biased’ library of binding sites which are engineered to span a large collection of sequences that present many combinations of mutations away from the target sequence the programmable DNA targeting complex was designed to bind. This is achieved using degenerate oligonucleotides engineered to bear nucleotide frequencies at each position such that the target sequence nucleotide appears at a 79% frequency and each other nucleotide occurs at 7% frequency. See Patwardhan et al., Nature Biotechnology 30, 265-270 (2012) hereby incorporated by reference in its entirety. The reporter library is then sequenced to reveal the associations between the 24 bp dTomato transcript tags and their corresponding ‘biased’ target site in the library element. The large diversity of the transcript tags assures that sharing of tags between different targets will be extremely rare, while the biased construction of the target sequences means that sites with few mutations will be associated with more tags than sites with more mutations. Next, transcription of the dTomato reporter genes is stimulated with either a control-TF engineered to bind the shared DNA site, or the target-TF that was engineered to bind the target site. The abundance of each expressed transcript tag is measured in each sample by conducting RNAseq on the stimulated cells, which is then mapped back to their corresponding binding sites using the association table established earlier. The control-TF is expected to excite all library members equally since its binding site is shared across all library elements, while the target-TF is expected to skew the distribution of the expressed members to those that are preferentially targeted by it. This assumption is used in step 5 to compute a normalized expression level for each binding site by dividing the tag counts obtained for the target-TF by those obtained for the control-TF.

As shown in FIG. 2B, the targeting landscape of a Cas9-gRNA complex reveals that it is on average tolerant to 1-3 mutations in its target sequences. As shown in FIG. 2C, the Cas9-gRNA complex is also largely insensitive to point mutations, except those localized to the PAM sequence. Notably this data reveals that the predicted PAM for the S. pyogenes Cas9 is not just NGG but also NAG. As shown in FIG. 2D, introduction of 2 base mismatches significantly impairs the Cas9-gRNA complex activity, however only when these are localized to the 8-10 bases nearer the 3′ end of the gRNA target sequence (in the heat plot the target sequence positions are labeled from 1-23 starting from the 5′ end).

The mutational tolerance of another widely used genome editing tool, TALE domains, was determined using the transcriptional specificity assay described herein. As shown in FIG. 2E, the TALE off-targeting data for an 18-mer TALE reveals that it can tolerate on average 1-2 mutations in its target sequence, and fails to activate a large majority of 3 base mismatch variants in its targets. As shown in FIG. 2F, the 18-mer TALE is, similar to the Cas9-gRNA complexes, largely insensitive to single base mismatched in its target. As shown in FIG. 2G, introduction of 2 base mismatches significantly impairs the 18-mer TALE activity. TALE activity is more sensitive to mismatches nearer the 5′ end of its target sequence (in the heat plot the target sequence positions are labeled from 1-18 starting from the 5′ end).

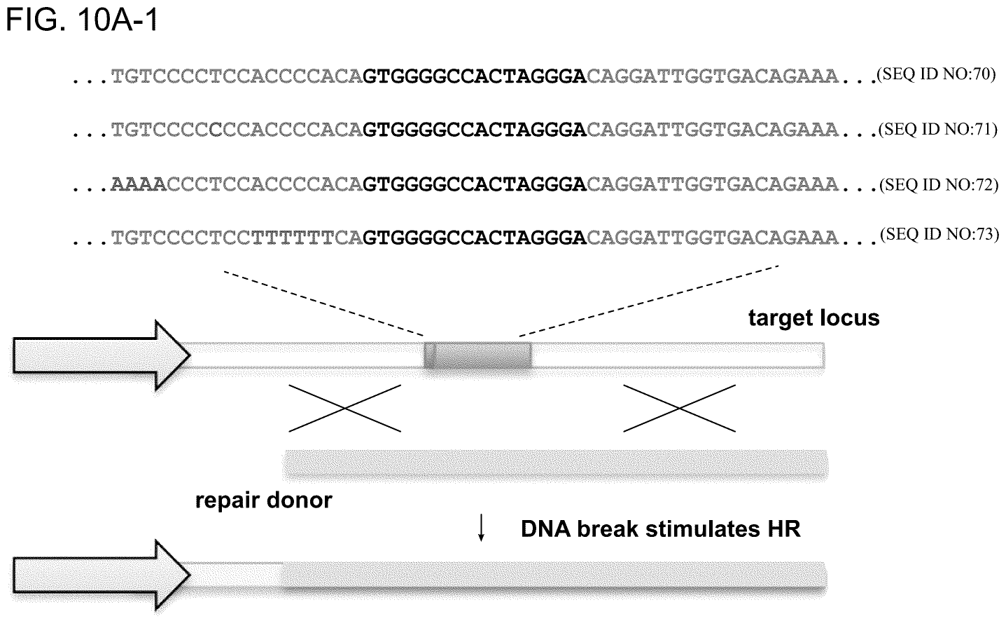

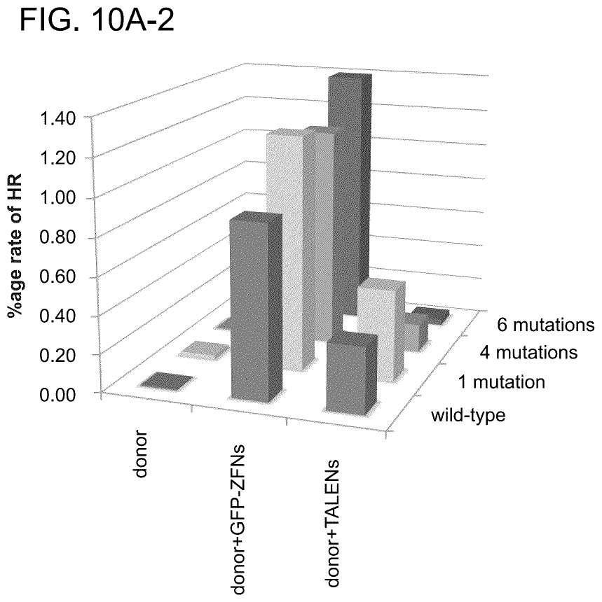

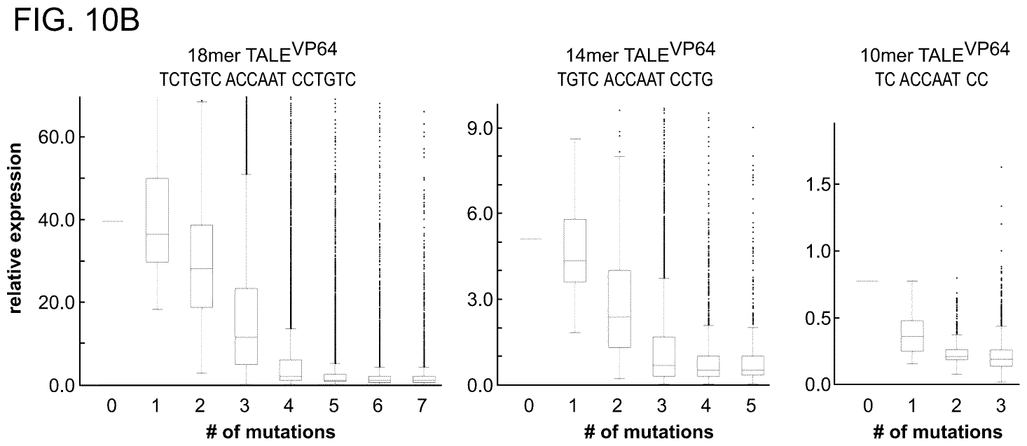

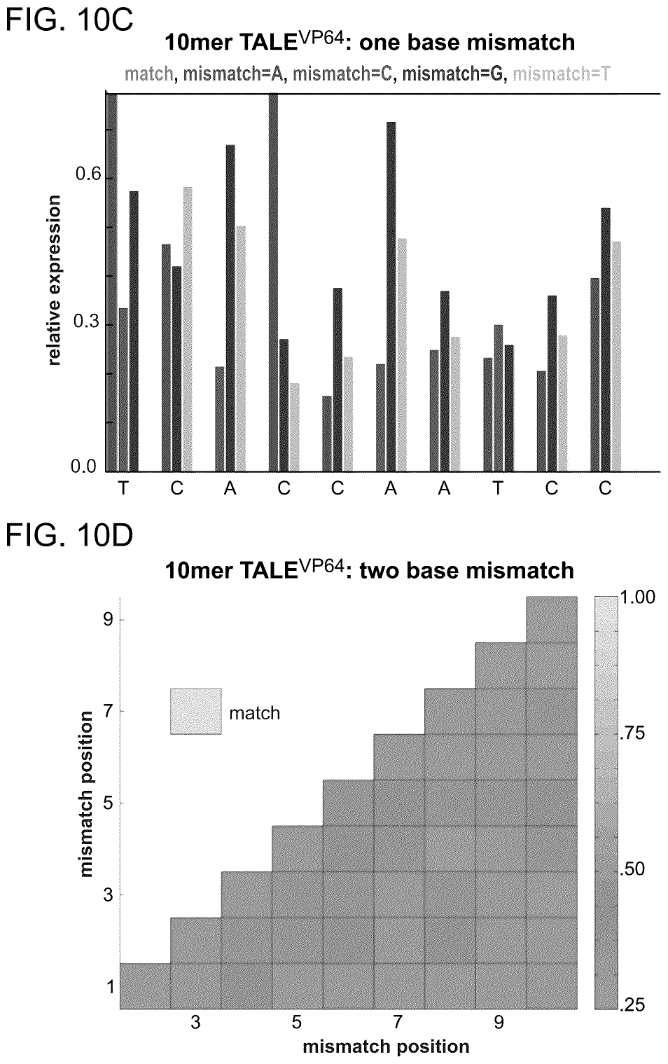

Results were confirmed using targeted experiments in a nuclease assay which is the subject of FIGS. 10A-C directed to evaluating the landscape of targeting by TALEs of different sizes. As shown in FIGS. 10A-1 and 10A-2, using a nuclease mediated HR assay, it was confirmed that 18-mer TALEs tolerate multiple mutations in their target sequences. As shown in FIG. 10B, using the approach described in FIG. 2, the targeting landscape of TALEs of 3 different sizes (18-mer, 14-mer and 10-mer) was analyzed. Shorter TALEs (14-mer and 10-mer) are progressively more specific in their targeting but also reduced in activity by nearly an order of magnitude. As shown in FIG. 10C and 10D, 10-mer TALEs show near single-base mismatch resolution, losing almost all activity against targets bearing 2 mismatches (in the heat plot the target sequence positions are labeled from 1-10 starting from the 5′ end). Taken together, these data imply that engineering shorter TALEs can yield higher specificity in genome engineering applications, while the requirement for FokI dimerization in TALE nuclease applications is essential to avoid off-target effect. Sec Kim et al., Proceedings of the National Academy of Sciences of the United States of America 93, 1156-1160 (1996) and Pattanayak et al., Nature Methods 8, 765-770 (2011) each of which are hereby incorporated by reference in its entirety.

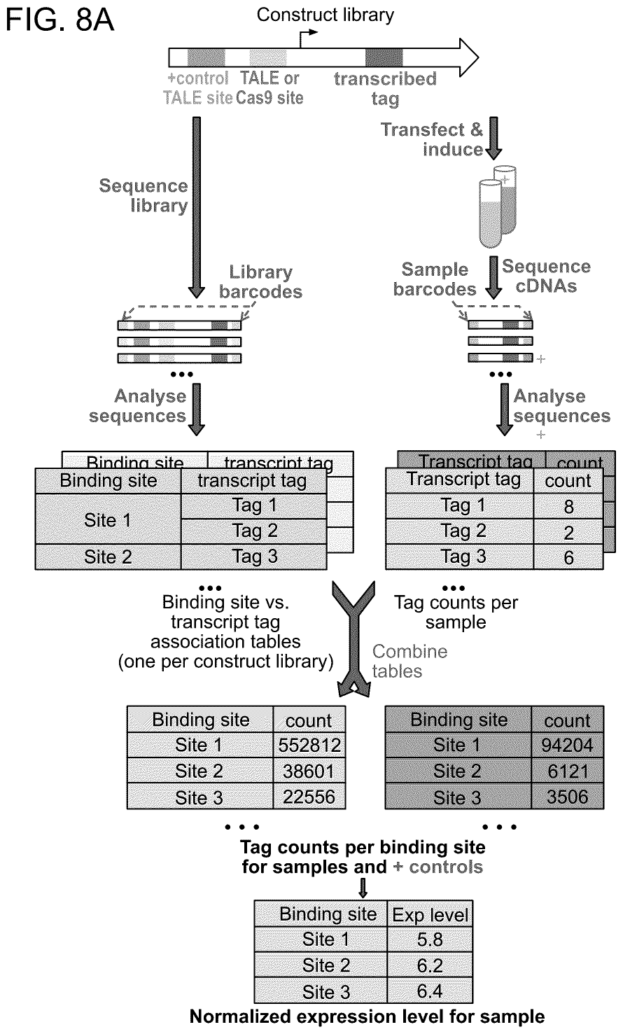

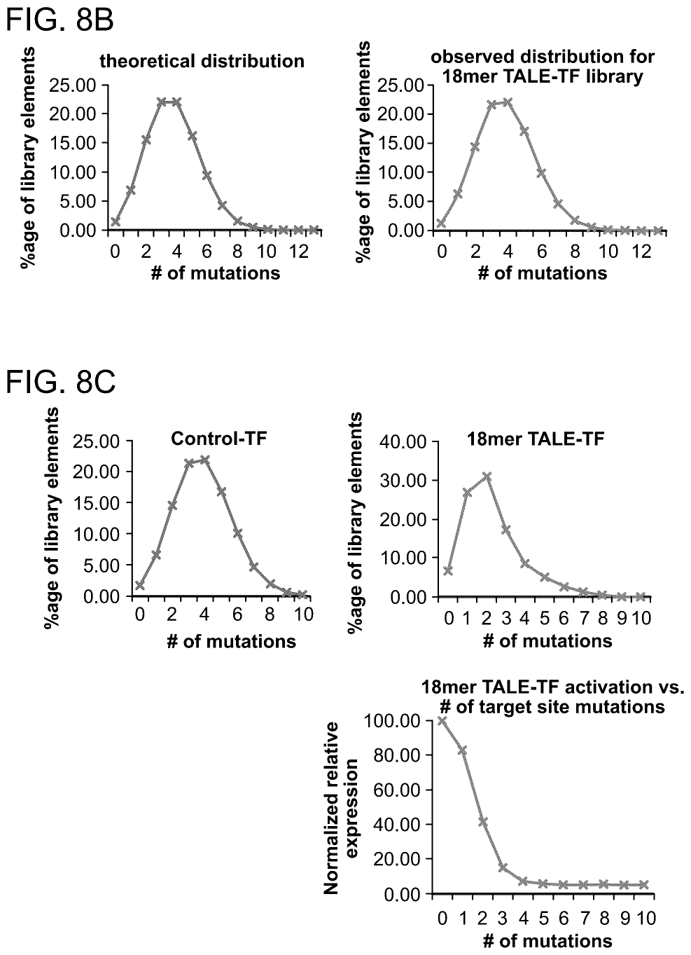

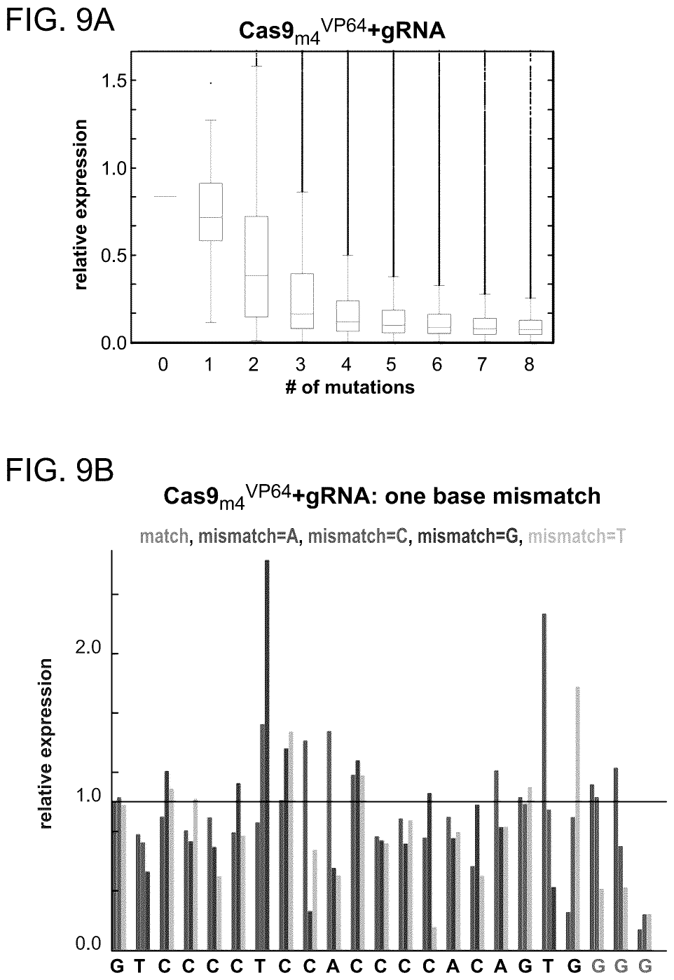

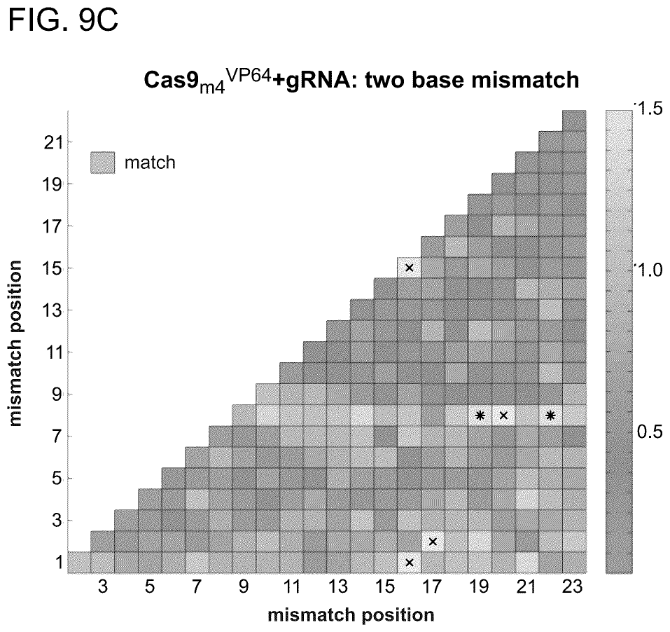

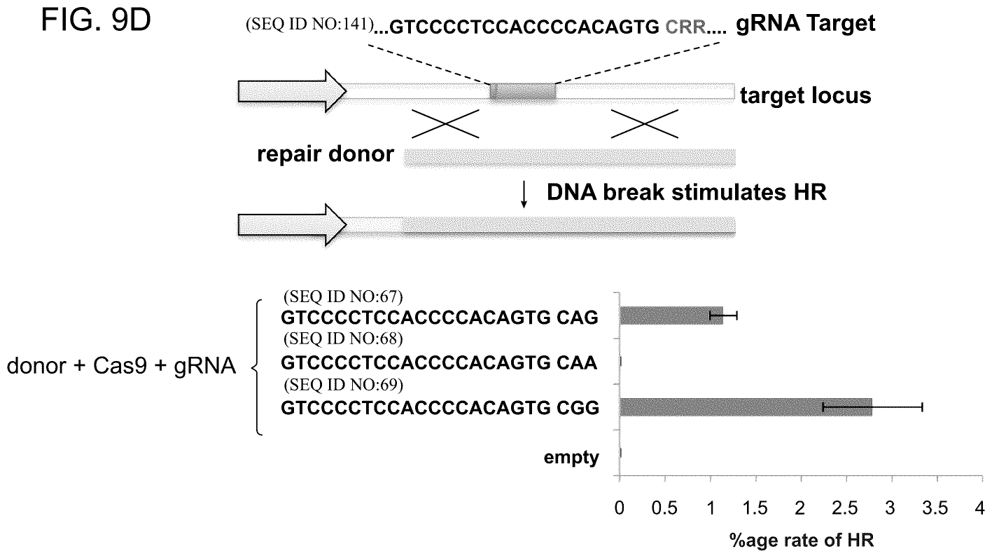

FIG. 8A-C is directed to high level specificity analysis processing flow for calculation of normalized expression levels illustrated with examples from experimental data. As shown in FIG. 8A. construct libraries are generated with a biased distribution of binding site sequences and random sequence 24 bp tags that will be incorporated into reporter gene transcripts (top). The transcribed tags are highly degenerate so that they should map many-to-one to Cas9 or TALE binding sequences. The construct libraries are sequenced (3^{rd} level, left) to establish which tags co-occur with binding sites, resulting in an association table of binding sites vs. transcribed tags (4^{th} level, left). Multiple construct libraries built for different binding sites may be sequenced at once using library barcodes (indicated here by the light blue and light yellow colors; levels 1-4, left). A construct library is then transfected into a cell population and a set of different Cas9/gRNA or TALE transcription factors are induced in samples of the populations (2^{nd} level, right). One sample is always induced with a fixed TALE activator targeted to a fixed binding site sequence within the construct (top level, green box); this sample serves as a positive control (green sample, also indicated by a +sign). cDNAs generated from the reporter mRNA molecules in the induced samples are then sequenced and analyzed to obtain tag counts for each tag in a sample (3^{rd} and 4^{th} level, right). As with the construct library sequencing, multiple samples, including the positive control, are sequenced and analyzed together by appending sample barcodes. Here the light red color indicates one non-control sample that has been sequenced and analyzed with the positive control (green). Because only the transcribed tags and not the construct binding sites appear in each read, the binding site vs. tag association table obtained from construct library sequencing is then used to tally up total counts of tags expressed from each binding site in each sample (5th level). The tallies for each non-positive control sample are then converted to normalized expression levels for each binding site by dividing them by the tallies obtained in the positive control sample. Examples of plots of normalized expression levels by numbers of mismatches are provided in FIGS. 2B and 2E, and in FIG. 9A and FIG. 10B. Not covered in this overall process flow are several levels of filtering for erroneous tags, for tags not associable with a construct library, and for tags apparently shared with multiple binding sites. FIG. 8B depicts example distributions of percentages of binding sites by numbers of mismatches generated within a biased construct library. Left: Theoretical distribution. Right: Distribution observed from an actual TALE construct library. FIG. 8C depicts example distributions of percentages of tag counts aggregated to binding sites by numbers of mismatches. Left: Distribution observed from the positive control sample. Right: Distribution observed from a sample in which a non-control TALE was induced. As the positive control TALE binds to a fixed site in the construct, the distribution of aggregated tag counts closely reflects the distribution of binding sites in FIG. 8B, while the distribution is skewed to the left for the non-control TALE sample because sites with fewer mismatches induce higher expression levels. Below: Computing the relative enrichment between these by dividing the tag counts obtained for the target-TF by those obtained for the control-TF reveals the average expression level versus the number of mutations in the target site.

These results are further reaffirmed by specificity data generated using a different Cas9-gRNA complex. As shown in FIG. 9A, a different Cas9-gRNA complex is tolerant to 1-3 mutations in its target sequence. As shown in FIG. 9B, the Cas9-gRNA complex is also largely insensitive to point mutations, except those localized to the PAM sequence. As shown in FIG. 9C, introduction of 2 base mismatches however significantly impairs activity (in the heat plot the target sequence positions are labeled from 1-23 starting from the 5′ end). As shown in FIG. 9D, it was confirmed using a nuclease mediated HR assay that the predicted PAM for the S. pyogenes Cas9 is NGG and also NAG.

According to certain aspects, binding specificity is increased according to methods described herein. Because synergy between multiple complexes is a factor in target gene activation by Cas9N-VP64, transcriptional regulation applications of Cas9N is naturally quite specific as individual off-target binding events should have minimal effect. According to one aspect, off-set nicks are used in methods of genome-editing. A large majority of nicks seldom result in NHEJ events, (see Certo et al., Nature Methods 8, 671-676 (2011) hereby incorporated by reference in its entirety) thus minimizing the effects of off-target nicking. In contrast, inducing off-set nicks to generate double stranded breaks (DSBs) is highly effective at inducing gene disruption. According to certain aspects, 5′ overhangs generate more significant NHEJ events as opposed to 3′ overhangs. Similarly, 3′ overhangs favor HR over NHEJ events, although the total number of HR events is significantly lower than when a 5′ overhang is generated. Accordingly, methods are provided for using nicks for homologous recombination and off-set nicks for generating double stranded breaks to minimize the effects of off-target Cas9-gRNA activity.

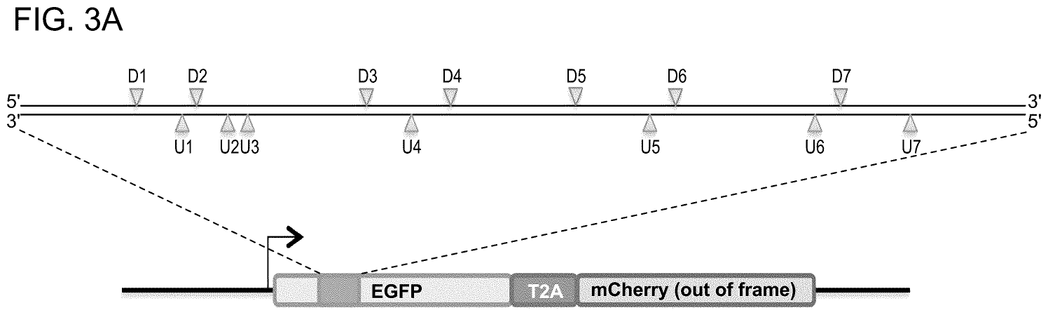

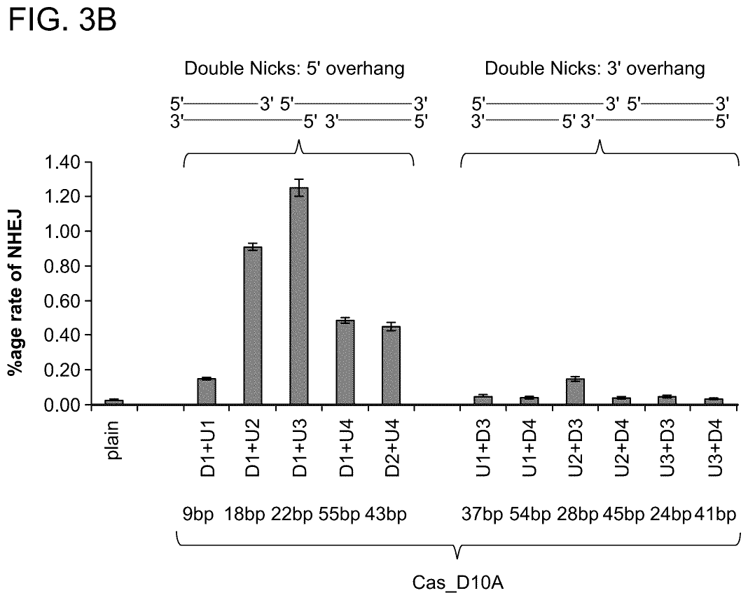

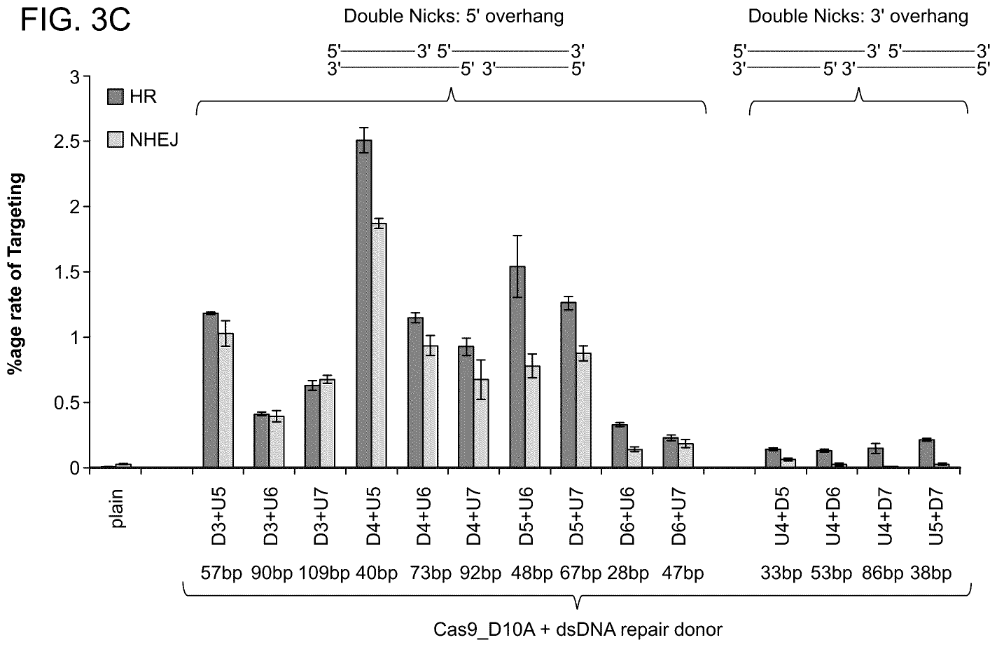

FIGS. 3A-C is directed to multiplex off-set nicking and methods for reducing the off-target binding with the guide RNAs. As shown in FIG. 3A, the traffic light reporter was used to simultaneously assay for HR and NHEJ events upon introduction of targeted nicks or breaks. DNA cleavage events resolved through the HDR pathway restore the GFP sequence, whereas mutagenic NHEJ causes frameshifts rendering the GFP out of frame and the downstream mCherry sequence in frame. For the assay, 14 gRNAs covering a 200 bp stretch of DNA: 7 targeting the sense strand (U1-7) and 7 the antisense strand (D1-7) were designed. Using the Cas9D10A mutant, which nicks the complementary strand, different two-way combinations of the gRNAs were used to induce a range of programmed 5′ or 3′ overhangs (the nicking sites for the 14 gRNAs are indicated). As shown in FIG. 3B, inducing off-set nicks to generate double stranded breaks (DSBs) is highly effective at inducing gene disruption. Notably off-set nicks leading to 5′ overhangs result in more NHEJ events as opposed to 3′ overhangs. As shown in FIG. 3C. generating 3′ overhangs also favors the ratio of HR over NHEJ events, but the total number of HR events is significantly lower than when a 5′ overhang is generated.

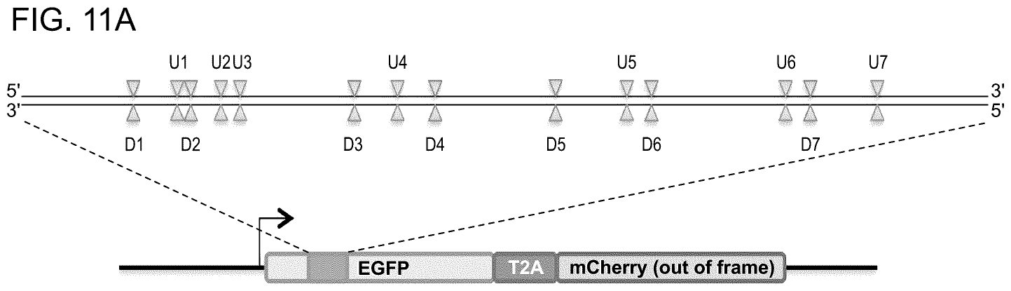

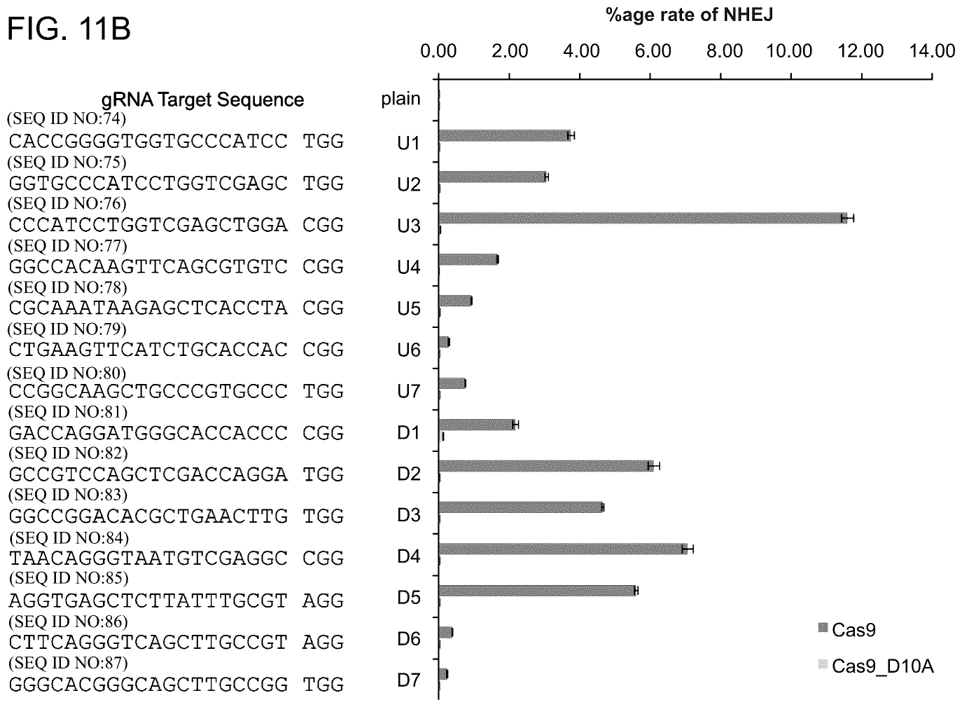

FIGS. 11A-B is directed to Cas9D10A nickase mediated NHEJ. As shown in FIG. 11A, the traffic light reporter was used to assay NHEJ events upon introduction of targeted nicks or double-stranded breaks. Briefly, upon introduction of DNA cleavage events, if the break goes through mutagenic NHEJ, the GFP is translated out of frame and the downstream mCherry sequences are rendered in frame resulting in red fluorescence. 14 gRNAs covering a 200 bp stretch of DNA: 7 targeting the sense strand (U1-7) and 7 the antisense strand (D1-7) were designed. As shown in FIG. 11B, it was observed that unlike the wild-type Cas9 which results in DSBs and robust NHEJ across all targets, most nicks (using the Cas9D10A mutant) seldom result in NHEJ events. All 14 sites are located within a contiguous 200 bp stretch of DNA and over 10-fold differences in targeting efficiencies were observed.

According to certain aspects, methods are described herein of modulating expression of a target nucleic acid in a cell that include introducing one or more, two or more or a plurality of foreign nucleic acids into the cell. The foreign nucleic acids introduced into the cell encode for a guide RNA or guide RNAs, a nuclease-null Cas9 protein or proteins and a transcriptional regulator protein or domain. Together. a guide RNA, a nuclease-null Cas9 protein and a transcriptional regulator protein or domain are referred to as a co-localization complex as that term is understood by one of skill in the art to the extent that the guide RNA, the nuclease-null Cas9 protein and the transcriptional regulator protein or domain bind to DNA and regulate expression of a target nucleic acid. According to certain additional aspects, the foreign nucleic acids introduced into the cell encode for a guide RNA or guide RNAs and a Cas9 protein nickase. Together, a guide RNA and a Cas9 protein nickase are referred to as a co-localization complex as that term is understood by one of skill in the art to the extent that the guide RNA and the Cas9 protein nickase bind to DNA and nick a target nucleic acid.

Cells according to the present disclosure include any cell into which foreign nucleic acids can be introduced and expressed as described herein. It is to be understood that the basic concepts of the present disclosure described herein are not limited by cell type. Cells according to the present disclosure include eukaryotic cells, prokaryotic cells, animal cells, plant cells, fungal cells, archael cells, eubacterial cells and the like. Cells include eukaryotic cells such as yeast cells, plant cells, and animal cells. Particular cells include mammalian cells. Further, cells include any in which it would be beneficial or desirable to regulate a target nucleic acid. Such cells may include those which are deficient in expression of a particular protein leading to a disease or detrimental condition. Such diseases or detrimental conditions are readily known to those of skill in the art. According to the present disclosure, the nucleic acid responsible for expressing the particular protein may be targeted by the methods described herein and a transcriptional activator resulting in upregulation of the target nucleic acid and corresponding expression of the particular protein. In this manner, the methods described herein provide therapeutic treatment.

Target nucleic acids include any nucleic acid sequence to which a co-localization complex as described herein can be useful to either regulate or nick. Target nucleic acids include genes. For purposes of the present disclosure, DNA, such as double stranded DNA, can include the target nucleic acid and a co-localization complex can bind to or otherwise co-localize with the DNA at or adjacent or near the target nucleic acid and in a manner in which the co-localization complex may have a desired effect on the target nucleic acid. Such target nucleic acids can include endogenous (or naturally occurring) nucleic acids and exogenous (or foreign) nucleic acids. One of skill based on the present disclosure will readily be able to identify or design guide RNAs and Cas9 proteins which co-localize to a DNA including a target nucleic acid. One of skill will further be able to identify transcriptional regulator proteins or domains which likewise co-localize to a DNA including a target nucleic acid. DNA includes genomic DNA, mitochondrial DNA, viral DNA or exogenous DNA.

Foreign nucleic acids (i.e. those which are not part of a cell's natural nucleic acid composition) may be introduced into a cell using any method known to those skilled in the art for such introduction. Such methods include transfection, transduction, viral transduction, microinjection, lipofection, nucleofection, nanoparticle bombardment, transformation, conjugation and the like. One of skill in the art will readily understand and adapt such methods using readily identifiable literature sources.

Transcriptional regulator proteins or domains which are transcriptional activators include VP16 and VP64 and others readily identifiable by those skilled in the art based on the present disclosure.

Diseases and detrimental conditions are those characterized by abnormal loss of expression of a particular protein. Such diseases or detrimental conditions can be treated by upregulation of the particular protein. Accordingly, methods of treating a disease or detrimental condition are provided where the co-localization complex as described herein associates or otherwise binds to DNA including a target nucleic acid, and the transcriptional activator of the co-localization complex upregulates expression of the target nucleic acid. For example upregulating PRDM16 and other genes promoting brown fat differentiation and increased metabolic uptake can be used to treat metabolic syndrome or obesity. Activating anti-inflammatory genes are useful in autoimmunity and cardiovascular disease. Activating tumor suppressor genes is useful in treating cancer. One of skill in the art will readily identify such diseases and detrimental conditions based on the present disclosure.

The following examples are set forth as being representative of the present disclosure. These examples are not to be construed as limiting the scope of the present disclosure as these and other equivalent embodiments will be apparent in view of the present disclosure, figures and accompanying claims.

### EXAMPLE I

### Cas9 Mutants

Sequences homologous to Cas9 with known structure were searched to identify candidate mutations in Cas9 that could ablate the natural activity of its RuvC and HNH domains. Using HHpred (world wide website toolkit.tuebingen.mpg.de/hhpred), the full sequence of Cas9 was queried against the full Protein Data Bank (January 2013). This search returned two different HNH endonucleases that had significant sequence homology to the HNH domain of Cas9; PacI and a putative endonuclease (PDB IDs: 3M7K and 4H9D respectively). These proteins were examined to find residues involved in magnesium ion coordination. The corresponding residues were then identified in the sequence alignment to Cas9. Two Mg-coordinating side-chains in each structure were identified that aligned to the same amino acid type in Cas9. They are 3M7K D92 and N113, and 4H9D D53 and N77. These residues corresponded to Cas9 D839 and N863. It was also reported that mutations of PacI residues D92 and N113 to alanine rendered the nuclease catalytically deficient. The Cas9 mutations D839A and N863A were made based on this analysis. Additionally, HHpred also predicts homology between Cas9 and the N-terminus of a Thermus thermophilus RuvC (PDB ID: 4EP4). This sequence alignment covers the previously reported mutation D10A which eliminates function of the RuvC domain in Cas9. To confirm this as an appropriate mutation, the metal binding residues were determined as before. In 4EP4, D7 helps to coordinate a magnesium ion. This position has sequence homology corresponding to Cas9 D10, confirming that this mutation helps remove metal binding, and thus catalytic activity from the Cas9 RuvC domain.

### EXAMPLE II

### Plasmid Construction

The Cas9 mutants were generated using the Quikchange kit (Agilent technologies). The target gRNA expression constructs were either (1) directly ordered as individual gBlocks from IDT and cloned into the pCR-BluntII-TOPO vector (Invitrogen); or (2) custom synthesized by Genewiz; or (3) assembled using Gibson assembly of oligonucleotides into the gRNA cloning vector (plasmid #41824). The vectors for the HR reporter assay involving a broken GFP were constructed by fusion PCR assembly of the GFP sequence bearing the stop codon and appropriate fragment assembled into the EGIP lentivector from Addgene (plasmid #26777). These lentivectors were then used to establish the GFP reporter stable lines. TALENs used in this study were constructed using standard protocols. See Sanjana et al., Nature Protocols 7, 171-192 (2012) hereby incorporated by reference in its entirety. Cas9N and MS2 VP64 fusions were performed using standard PCR fusion protocol procedures. The promoter luciferase constructs for OCT4 and REX1 were obtained from Addgene (plasmid #17221 and plasmid #17222).

### EXAMPLE III

### Cell Culture and Transfections

HEK 293T cells were cultured in Dulbecco's modified Eagle's medium (DMEM, Invitrogen) high glucose supplemented with 10% fetal bovine serum (FBS, Invitrogen), penicillin/streptomycin (pen/strep, Invitrogen), and non-essential amino acids (NEAA, Invitrogen). Cells were maintained at 37° ° C.and 5% CO₂ in a humidified incubator.

Transfections involving nuclease assays were as follows: 0.4×10⁶ cells were transfected with 2 μg Cas9 plasmid, 2 μg gRNA and/or 2 μg DNA donor plasmid using Lipofectamine 2000 as per the manufacturer's protocols. Cells were harvested 3 days after transfection and either analyzed by FACS, or for direct assay of genomic cuts the genomic DNA of ˜1×10⁶ cells was extracted using DNAeasy kit (Qiagen). For these PCR was conducted to amplify the targeting region with genomic DNA derived from the cells and amplicons were deep sequenced by MiSeq Personal Sequencer (Illumina) with coverage >200,000 reads. The sequencing data was analyzed to estimate NHEJ efficiencies.

For transfections involving transcriptional activation assays: 0.4×10⁶ cells were transfected with (1) 2 μg Cas9N-VP64 plasmid, 2 μg gRNA and/or 0.25 μg of reporter construct: or (2) 2 μg Cas9N plasmid, 2 μg MS2-VP64, 2 μg gRNA-2XMS2aptamer and/or 0.25 μg of reporter construct. Cells were harvested 24-48 hrs post transfection and assayed using FACS or immunofluorescence methods, or their total RNA was extracted and these were subsequently analyzed by RT-PCR. Here standard taqman probes from Invitrogen for OCT4 and REX1 were used, with normalization for each sample performed against GAPDH.

For transfections involving transcriptional activation assays for specificity profile of Cas9-gRNA complexes and TALEs: 0.4×10⁶ cells were transfected with (1) 2 μg Cas9N-VP64 plasmid, 2 μg gRNA and 0.25 μg of reporter library: or (2) 2 μg TALE-TF plasmid and 0.25 μg of reporter library: or (3) 2 μg control-TF plasmid and 0.25 μg of reporter library. Cells were harvested 24 hrs post transfection (to avoid the stimulation of reporters being in saturation mode). Total RNA extraction was performed using RNAeasy-plus kit (Qiagen), and standard RT-pcr performed using Superscript-III (Invitrogen). Libraries for next-generation sequencing were generated by targeted per amplification of the transcript-tags.

### EXAMPLE IV

### Computational and Sequence Analysis for Calculation of Cas9-TF and TALE-TF Reporter Expression Levels

The high-level logic flow for this process is depicted in FIG. 8A, and additional details are given here. For details on construct library composition, see FIGS. 8A (level 1) and 8B.

Sequencing: For Cas9 experiments, construct library (FIG. 8A, level 3, left) and reporter gene cDNA sequences (FIG. 8A, level 3, right) were obtained as 150 bp overlapping paired end reads on an Illumina MiSeq, while for TALE experiments, corresponding sequences were obtained as 51 bp non-overlapping paired end reads on an Illumina HiSeq.
Construct library sequence processing: Alignment: For Cas9 experiments, novoalign V2.07.17 (world wide website novocraft.com/main/index/php) was used to align paired reads to a set of 250 bp reference sequences that corresponded to 234 bp of the constructs flanked by the pairs of 8 bp library barcodes (see FIG. 8A, 3^{rd} level, left). In the reference sequences supplied to novoalign, the 23 bp degenerate Cas9 binding site regions and the 24 bp degenerate transcript tag regions (see FIG. 8A, first level) were specified as Ns, while the construct library barcodes were explicitly provided. For TALE experiments, the same procedures were used except that the reference sequences were 203 bp in length and the degenerate binding site regions were 18 bp vs. 23 bp in length. Validity checking: Novoalign output for comprised files in which left and right reads for each read pair were individually aligned to the reference sequences. Only read pairs that were both uniquely aligned to the reference sequence were subjected to additional validity conditions, and only read pairs that passed all of these conditions were retained. The validity conditions included: (i) Each of the two construct library barcodes must align in at least 4 positions to a reference sequence barcode. and the two barcodes must to the barcode pair for the same construct library. (ii) All bases aligning to the N regions of the reference sequence must be called by novoalign as As, Cs, Gs or Ts. Note that for neither Cas9 nor TALE experiments did left and right reads overlap in a reference N region, so that the possibility of ambiguous novoalign calls of these N bases did not arise. (iii) Likewise, no novoalign-called inserts or deletions must appear in these regions. (iv) No Ts must appear in the transcript tag region (as these random sequences were generated from As, Cs, and Gs only). Read pairs for which any one of these conditions were violated were collected in a rejected read pair file. These validity checks were implemented using custom perl scripts.
Induced sample reporter gene cDNA sequence processing: Alignment: SeqPrep (downloaded from world wide website github.com/jstjohn/SeqPrep) was first used to merge the overlapping read pairs to the 79 bp common segment, after which novoalign (version above) was used to align these 79 bp common segments as unpaired single reads to a set of reference sequences (see FIG. 8A, 3^{rd} level, right) in which (as for the construct library sequencing) the 24 bp degenerate transcript tag was specified as Ns while the sample barcodes were explicitly provided. Both TALE and Cas9 cDNA sequence regions corresponded to the same 63 bp regions of cDNA flanked by pairs of 8 bp sample barcode sequences. Validity checking: The same conditions were applied as for construct library sequencing (see above) except that: (a) Here, due prior SeqPrep merging of read pairs, validity processing did not have to filter for unique alignments of both reads in a read pair but only for unique alignments of the merged reads. (b) Only transcript tags appeared in the cDNA sequence reads, so that validity processing only applied these tag regions of the reference sequences and not also to a separate binding site region.
Assembly of table of binding sites vs. transcript tag associations: Custom perl was used to generate these tables from the validated construct library sequences (FIG. 8A, 4^{th} level, left). Although the 24 bp tag sequences composed of A. C. and G bases should be essentially unique across a construct library (probability of sharing =˜2.8e-11), carly analysis of binding site vs. tag associations revealed that a non-negligible fraction of tag sequences were in fact shared by multiple binding sequences, likely mainly caused by a combination of sequence errors in the binding sequences, or oligo synthesis errors in the oligos used to generate the construct libraries. In addition to tag sharing, tags found associated with binding sites in validated read pairs might also be found in the construct library read pair reject file if it was not clear, due to barcode mismatches, which construct library they might be from. Finally, the tag sequences themselves might contain sequence errors. To deal with these sources of error, tags were categorized with three attributes: (i) safe vs. unsafe, where unsafe meant the tag could be found in the construct library rejected read pair file; shared vs. nonshared, where shared meant the tag was found associated with multiple binding site sequences, and 2+ vs. 1-only, where 2+ meant that the tag appeared at least twice among the validated construct library sequences and so presumed to be less likely to contain sequence errors. Combining these three criteria yielded 8 classes of tags associated with each binding site, the most secure (but least abundant) class comprising only safe, nonshared, 2+ tags; and the least secure (but most abundant) class comprising all tags regardless of safety, sharing, or number of occurrences.
Computation of normalized expression levels: Custom perl code was used to implement the steps indicated in FIG. 8A, levels 5-6. First, tag counts obtained for each induced sample were aggregated for cach binding site, using the binding site vs. transcript tag table previously computed for the construct library (see FIG. 8C). For each sample, the aggregated tag counts for each binding site were then divided by the aggregated tag counts for the positive control sample to generate normalized expression levels. Additional considerations relevant to these calculations included:
1. For each sample, a subset of “novel” tags were found among the validity-checked cDNA gene sequences that could not be found in the binding site vs. transcript tag association table. These tags were ignored in the subsequent calculations.
2. The aggregations of tag counts described above were performed for each of the eight classes of tags described above in binding site vs. transcript tag association table. Because the binding sites in the construct libraries were biased to generate sequences similar to a central sequence frequently, but sequences with increasing numbers of mismatches increasingly rarely, binding sites with few mismatches generally aggregated to large numbers of tags, while binding sites with more mismatches aggregated to smaller numbers. Thus, although use of the most secure tag class was generally desirable, evaluation of binding sites with two or more mismatches might be based on small numbers of tags per binding site, making the secure counts and ratios less statistically reliable even if the tags themselves were more reliable. In such cases, all tags were used. Some compensation for this consideration obtains from the fact that the number of separate aggregated tag counts for n mismatching positions grew with the number of combinations of mismatching positions (equal to (ₙ^{L})3ⁿ), and so dramatically increases with n; thus the averages of aggregated tag counts for different numbers n of mismatches (shown in FIGS. 2b, 2e, and in FIGS. 9A and 10B) are based on a statistically very large set of aggregated tag counts for n≥2.
3. Finally, the binding site built into the TALE construct libraries was 18 bp and tag associations were assigned based on these 18 bp sequences, but some experiments were conducted with TALEs programmed to bind central 14 bp or 10 bp regions within the 18 bp construct binding site regions. In computing expression levels for these TALEs, tags were aggregated to binding sites based on the corresponding regions of the 18 bp binding sites in the association table, so that binding site mismatches outside of this region were ignored.

### EXAMPLE V

### RNA-guided SOX2 and NANOG Regulation Using Cas9N VP64

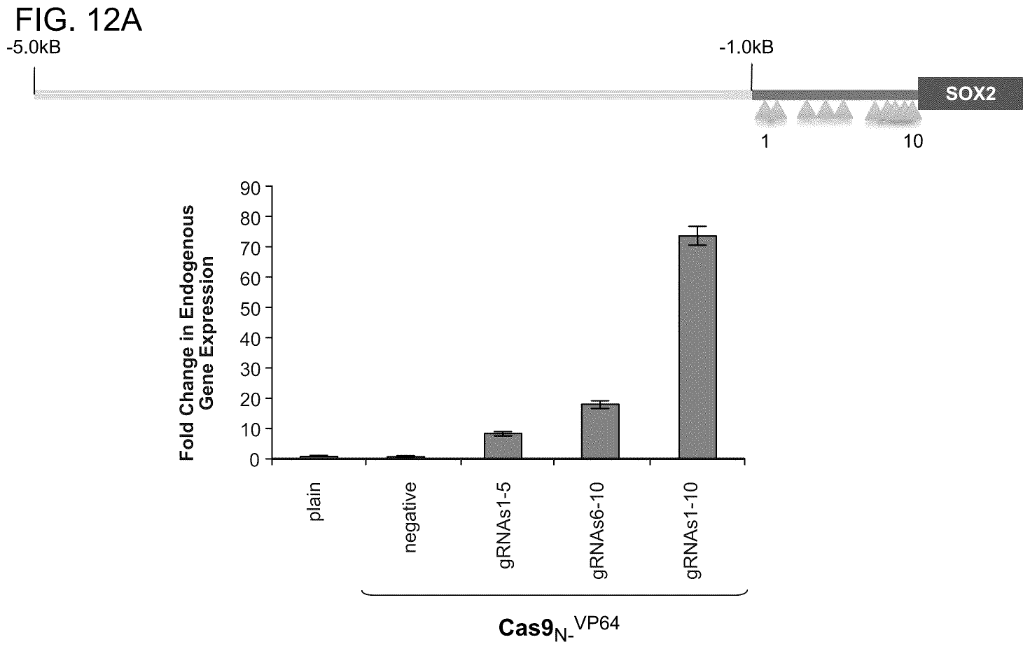

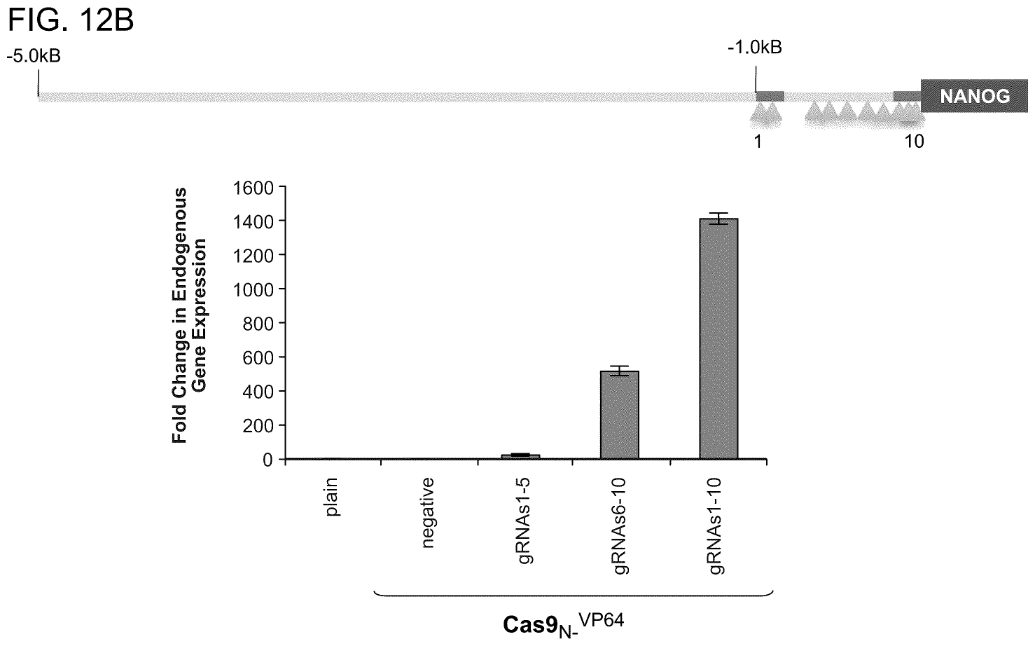

The sgRNA (aptamer-modified single guide RNA) tethering approach described herein allows different effector domains to be recruited by distinct sgRNAs so long as each sgRNA uses a different RNA-protein interaction pair, enabling multiplex gene regulation using the same Cas9N- protein. For the FIG. 12A SOX2 and FIG. 12B NANOG genes, 10 gRNAs were designed targeting a ˜1 kb stretch of DNA upstream of the transcription start site. The DNase hypersensitive sites are highlighted in green. Transcriptional activation via qPCR of the endogenous genes was assayed. In both instances, while introduction of individual gRNAs modestly stimulated transcription, multiple gRNAs acted synergistically to stimulate robust multi-fold transcriptional activation. Data are means +/− SEM (N=3). As shown in FIGS. 12A-B, two additional genes, SOX2 and NANOG, were regulated via sgRNAs targeting within an upstream ˜1kb stretch of promoter DNA. The sgRNAs proximal to the transcriptional start site resulted in robust gene activation.

### EXAMPLE VI

### Evaluating the Landscape of targeting by Cas9-gRNA Complexes

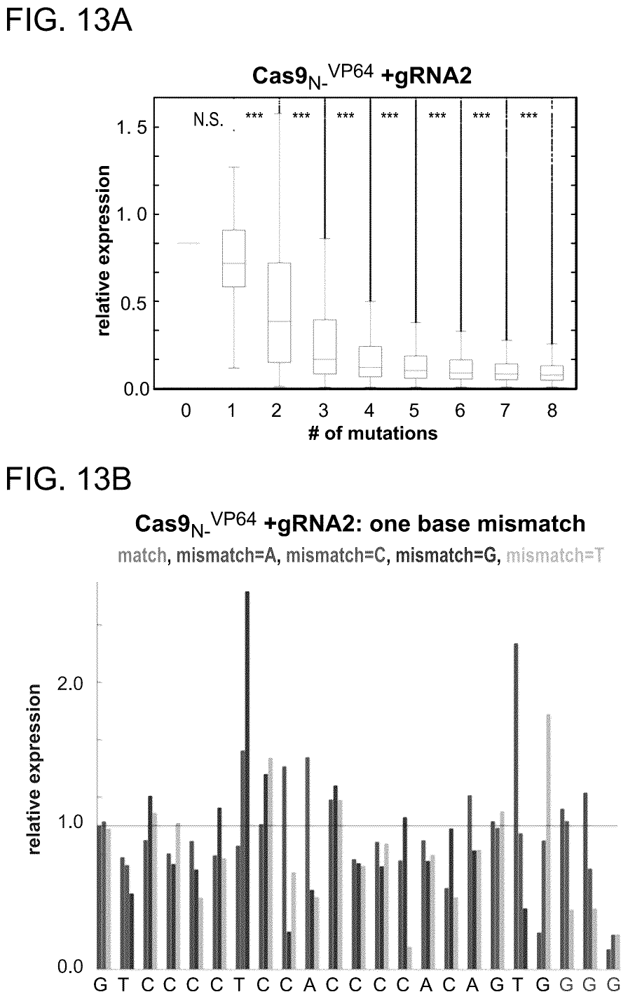

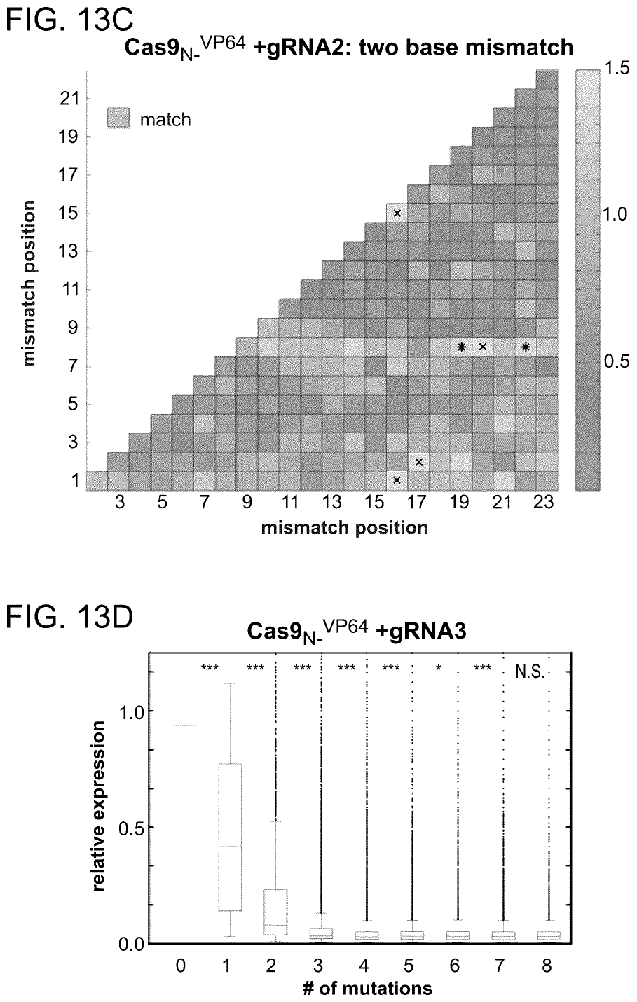

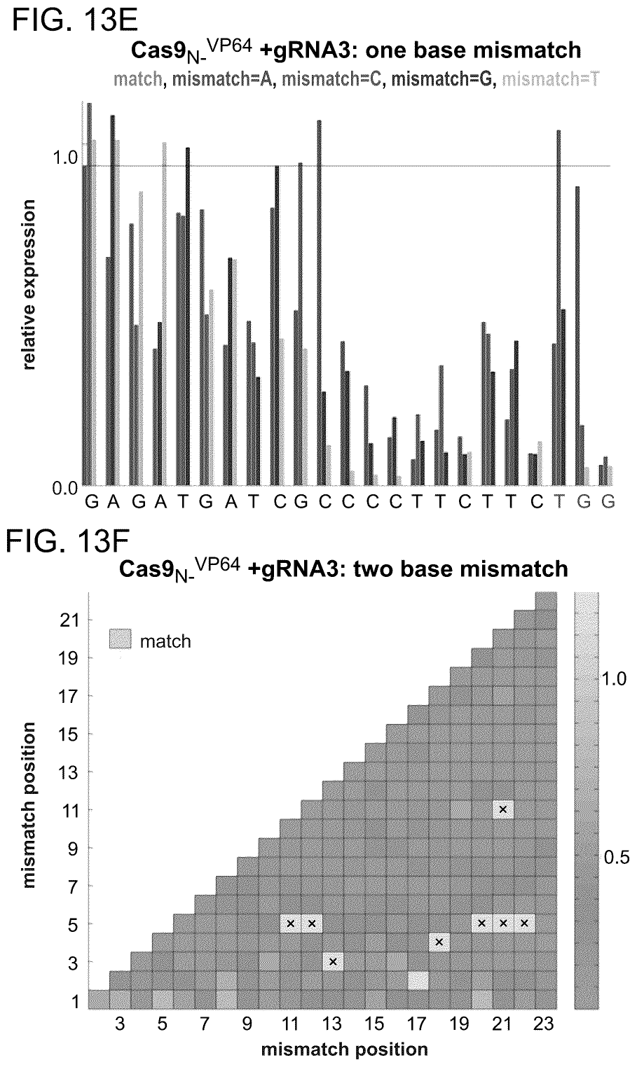

Using the approach described in FIG. 2, the targeting landscape of two additional Cas9-gRNA complexes (FIGS. 13A-C) and (FIGS. 13D-F) was analyzed. The two gRNAs have vastly different specificity profiles with gRNA2 tolerating up to 2-3 mismatches and gRNA3 only up to 1. These aspects are reflected in both the one base mismatch (FIGS. 13B, 13E) and two base mismatch plots (FIGS. 13C, 13F). In FIGS. 13C and 13F, base mismatch pairs for which insufficient data were available to calculate a normalized expression level are indicated as gray boxes containing an ‘x’, while, to improve data display, mismatch pairs whose normalized expression levels are outliers that exceed the top of the color scale are indicated as yellow boxes containing an asterisk ‘*’. Statistical significance symbols are: *** for P <. 0005/n, ** for P<0.005/n, * for P>0.05/n, and N.S. (Non-Significant) for P>=0.05/n, where n is the number of comparisons (refer Table 2).

### EXAMPLE VII

### Validations, Specificity of Reporter Assay

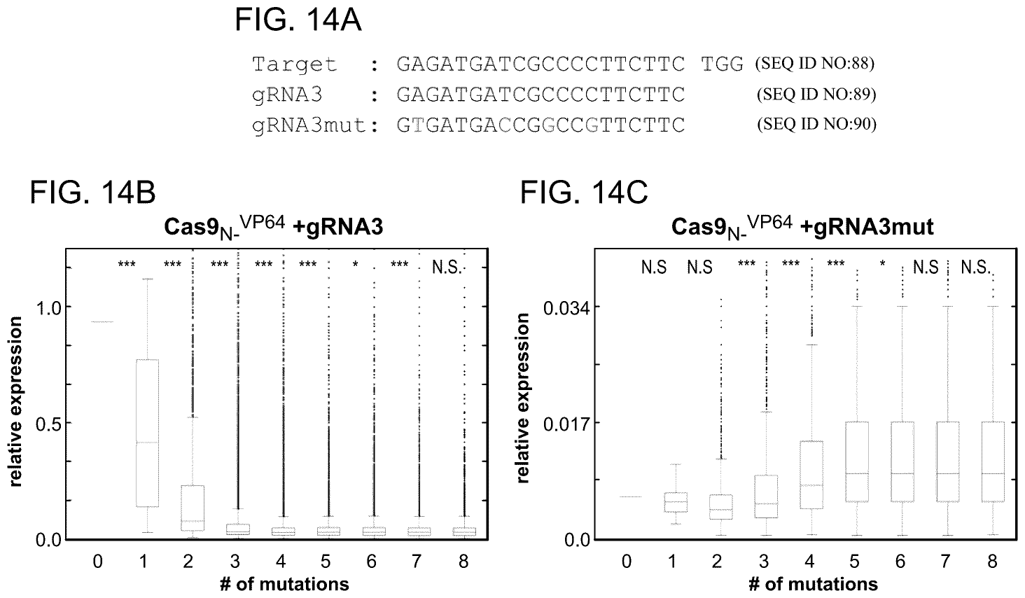

As shown in FIGS. 14A-C, specificity data was generated using two different sgRNA:Cas9 complexes. It was confirmed that the assay was specific for the sgRNA being evaluated, as a corresponding mutant sgRNA was unable to stimulate the reporter library. FIG. 14A: The specificity profile of two gRNAs (wild-type and mutant; sequence differences are highlighted in red) were evaluated using a reporter library designed against the wild-type gRNA target sequence. FIG. 14B: It was confirmed that this assay was specific for the gRNA being evaluated (data re-plotted from FIG. 13D), as the corresponding mutant gRNA is unable to stimulate the reporter library. Statistical significance symbols are: *** for P<0.0005/n, ** for P<0.005/n, * for P<0.05/n, and N.S. (Non-Significant) for P>=0.05/n, where n is the number of comparisons (refer Table 2). Different sgRNAs can have different specificity profiles (FIGS. 13A, 13D), specifically, sgRNA2 tolerates up to 3 mismatches and sgRNA3 only up to 1. The greatest sensitivity to mismatches was localized to the 3′ end of the spacer, albeit mismatches at other positions were also observed to affect activity.

### EXAMPLE VIII

### Validations, Single and Double-base gRNA Mismatches

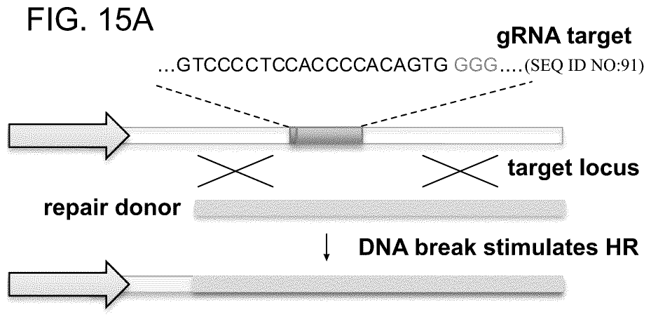

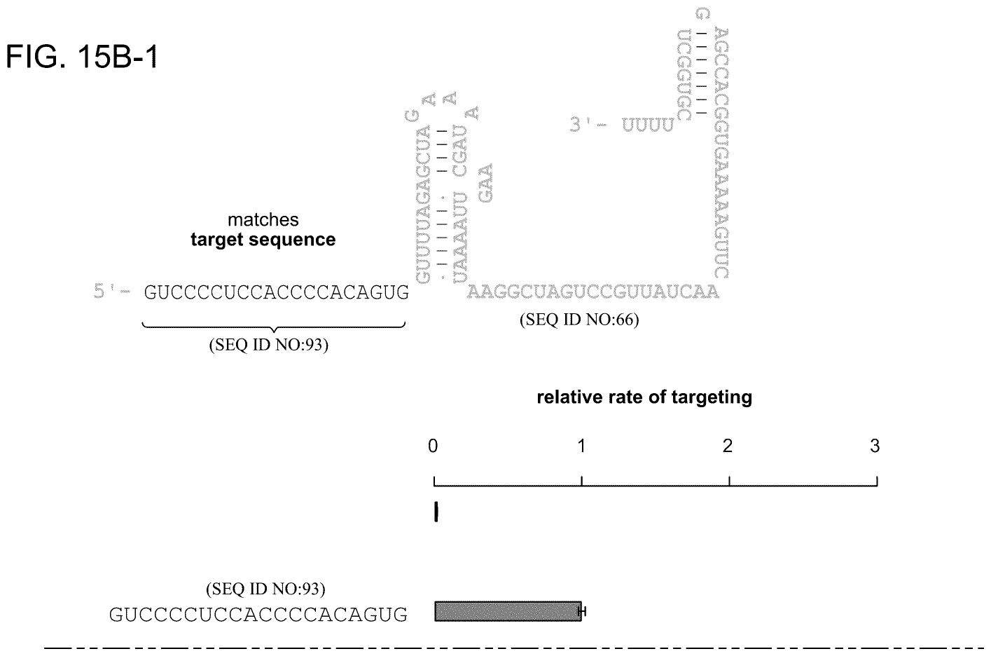

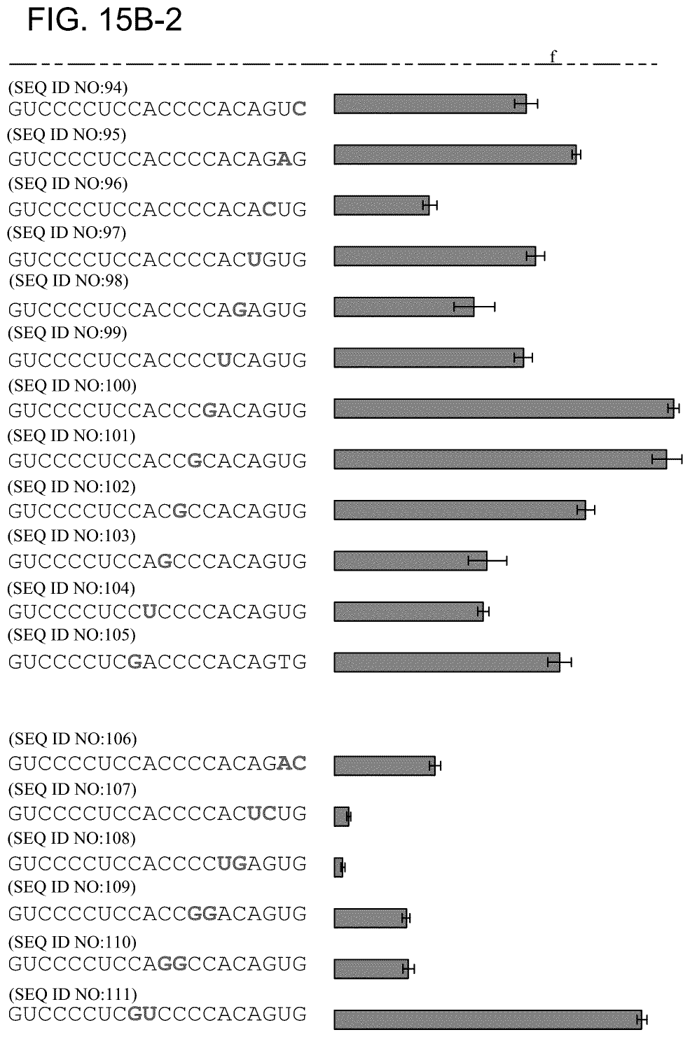

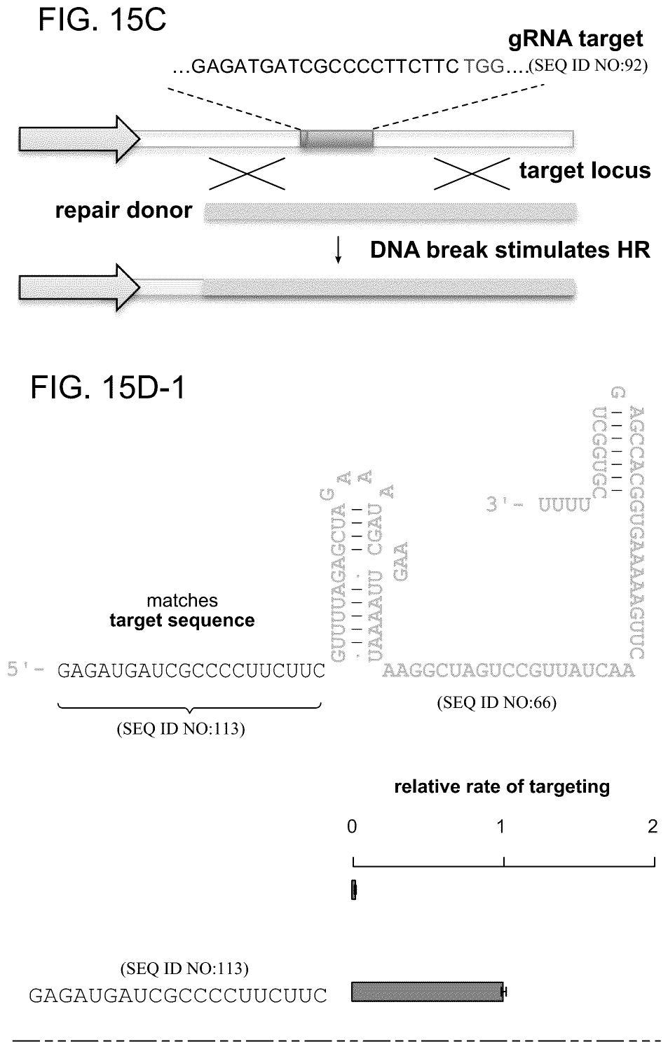

As shown in FIGS. 15A, 15B-1, 15B-2, 15C, 15D-1, and 15D-2, it was confirmed by targeted experiments that single-base mismatches within 12 bp of the 3′ end of the spacer in the assayed sgRNAs resulted in detectable targeting. However, 2 bp mismatches in this region resulted in significant loss of activity. Using a nuclease assay, 2 independent gRNAs were tested: gRNA2 (FIGS. 15A-15B-2) and gRNA3 (FIGS. 15C-15D-2) bearing single or double-base mismatches (highlighted in red) in the spacer sequence versus the target. It was confirmed that single-base mismatches within 12 bp of the 3′ end of the spacer in the assayed gRNAs result in detectable targeting, however 2 bp mismatches in this region result in rapid loss of activity. These results further highlight the differences in specificity profiles between different gRNAs consistent with the results in FIG. 13. Data are means +/− SEM (N=3).

### EXAMPLE IX

### Validations, 5′ gRNA truncations

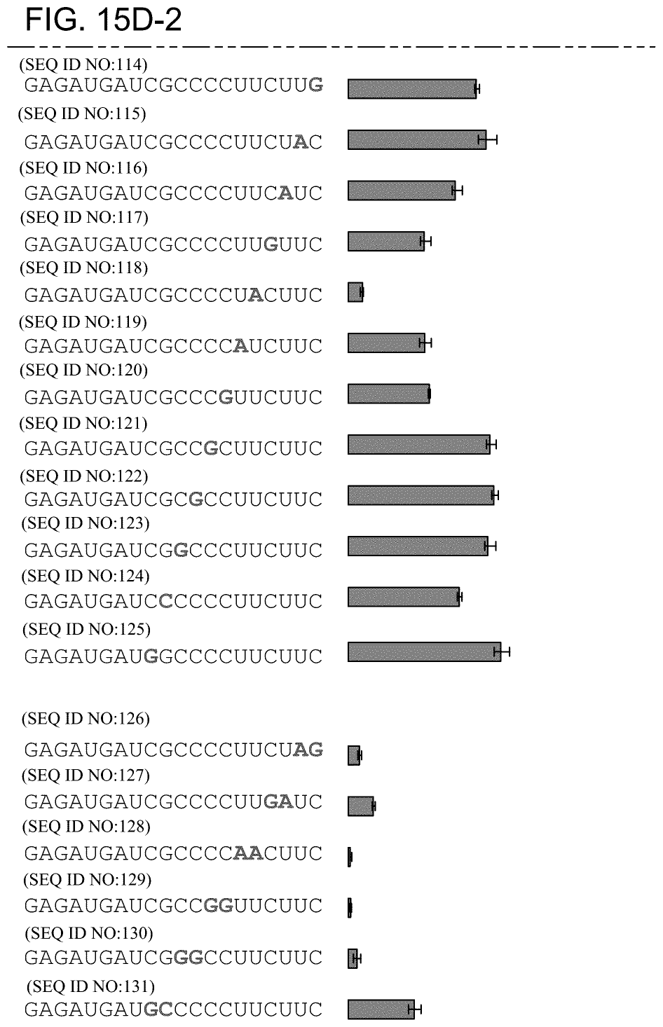

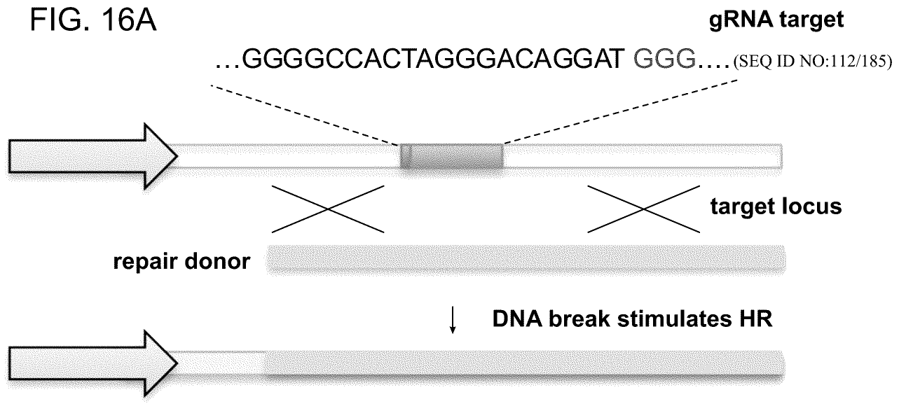

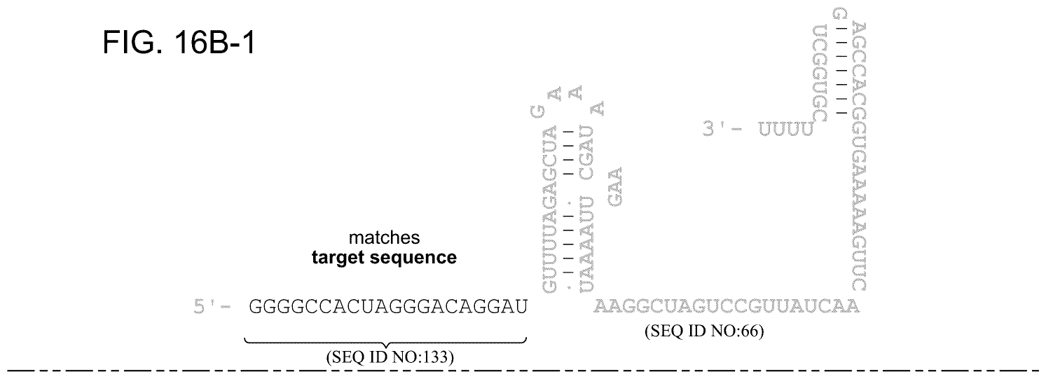

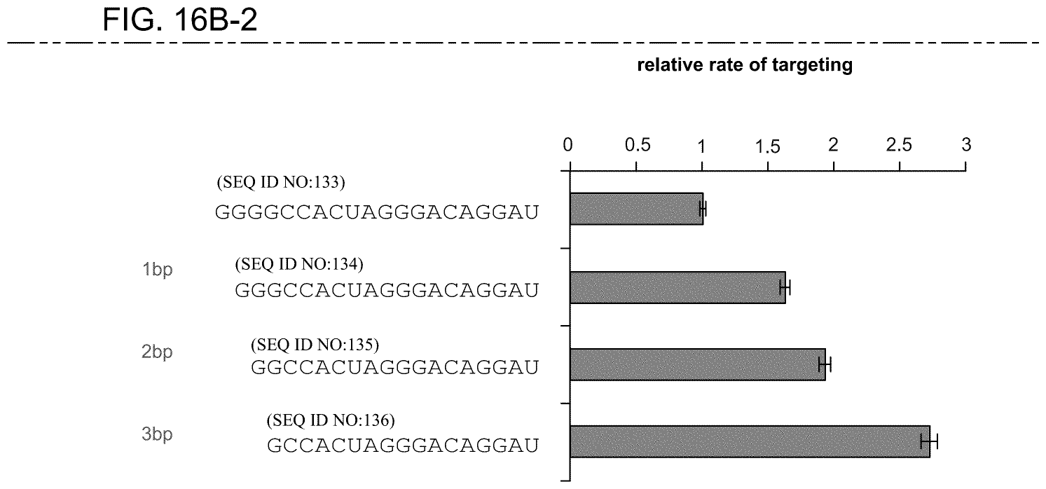

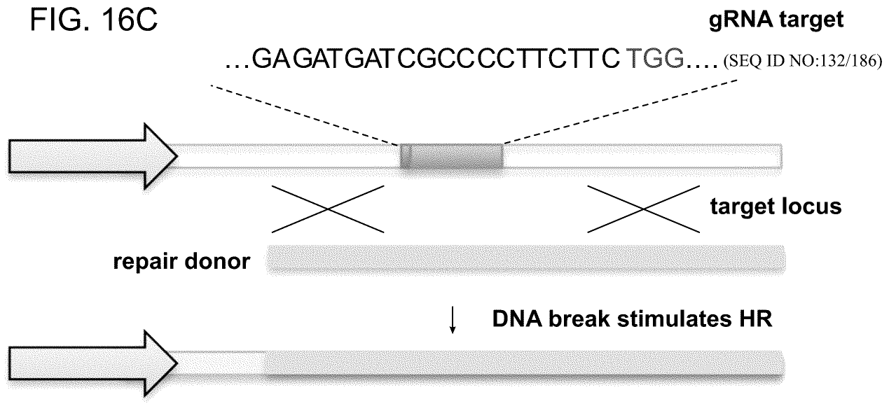

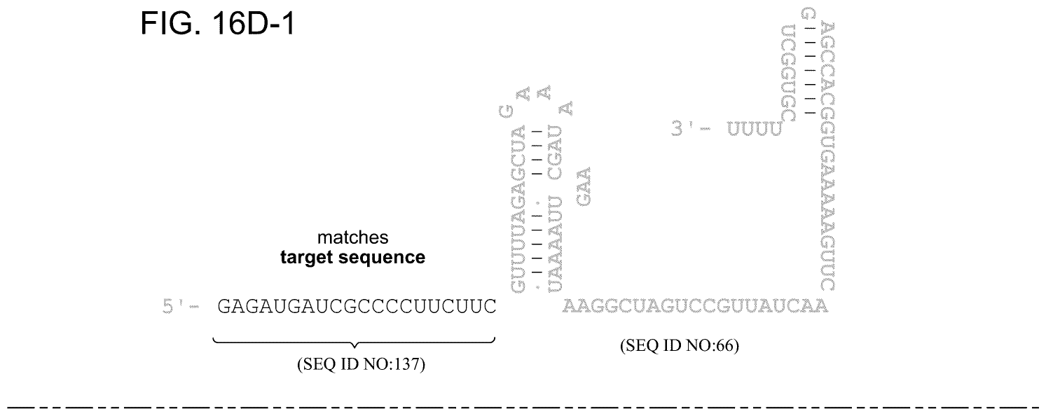

As shown in FIGS. 16A, 16B-1, 16B-2, 16C, 16D-1, and 16D-2, truncations in the 5′ portion of the spacer resulted in retention of sgRNA activity. Using a nuclease assay, 2 independent gRNA were tested: gRNA1 (FIGS. 16A-16B-2) and gRNA3 (FIGS. 16C-16D-2) bearing truncations at the 5′ end of their spacer. It was observed that 1-3 bp 5′ truncations are well tolerated, but larger deletions lead to loss of activity. Data are means +/− SEM (N=3).

### EXAMPLE X

### Validations, S. pyogenes PAM

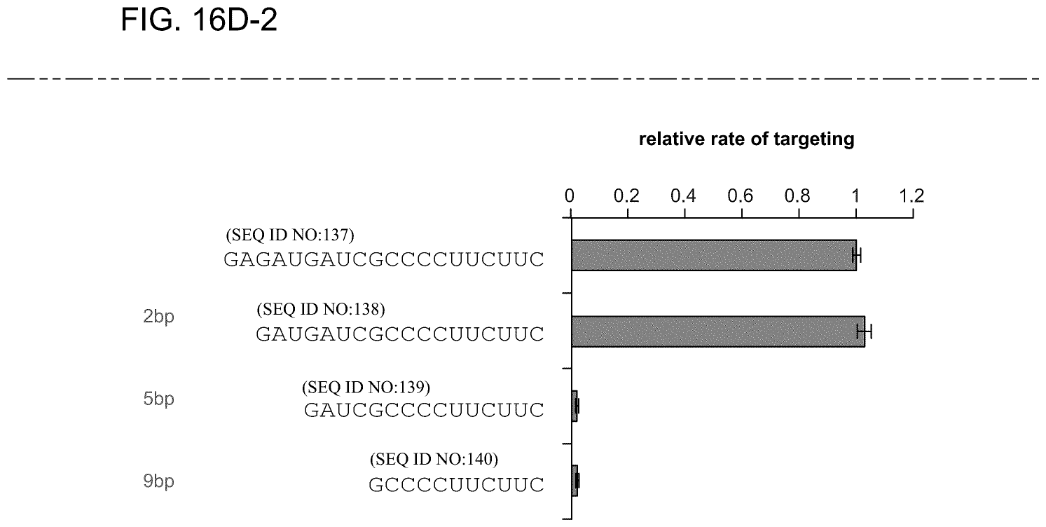

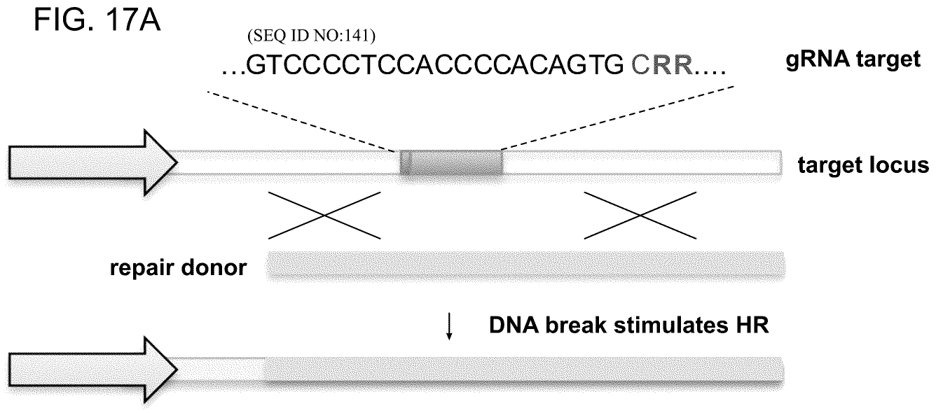

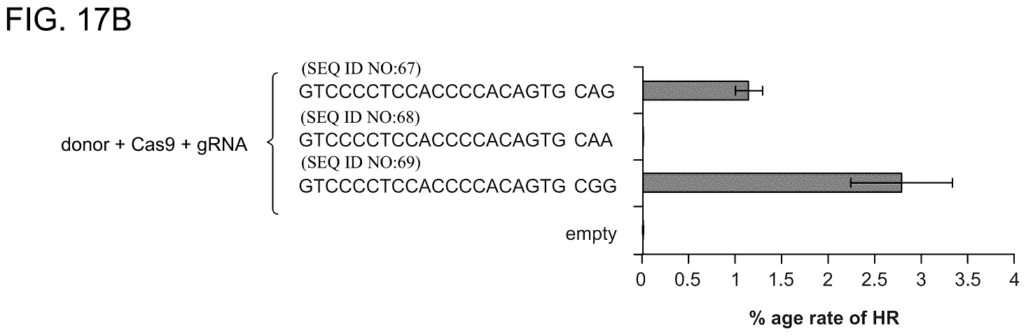

As shown in FIGS. 17A-B, it was confirmed using a nuclease mediated HR assay that the PAM for the S. pyogenes Cas9 is NGG and also NAG. Data are means +/− SEM (N=3). According to an additional investigation, a generated set of about 190K Cas9 targets in human exons that had no alternate NGG targets sharing the last 13 nt of the targeting sequence was scanned for the presence of alternate NAG sites or for NGG sites with a mismatch in the prior 13 nt. Only 0.4% were found to have no such alternate targets.

### EXAMPLE XI

### Validations, TALE Mutations

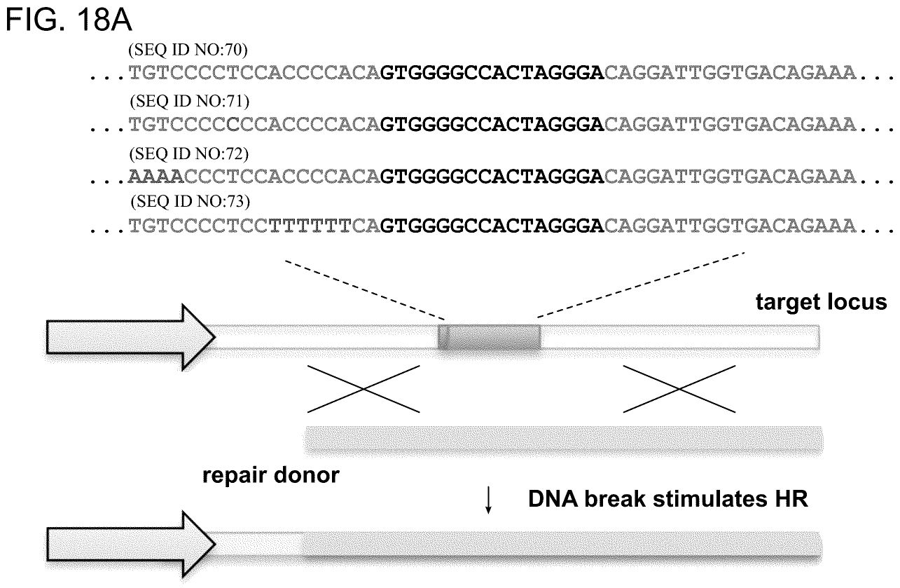

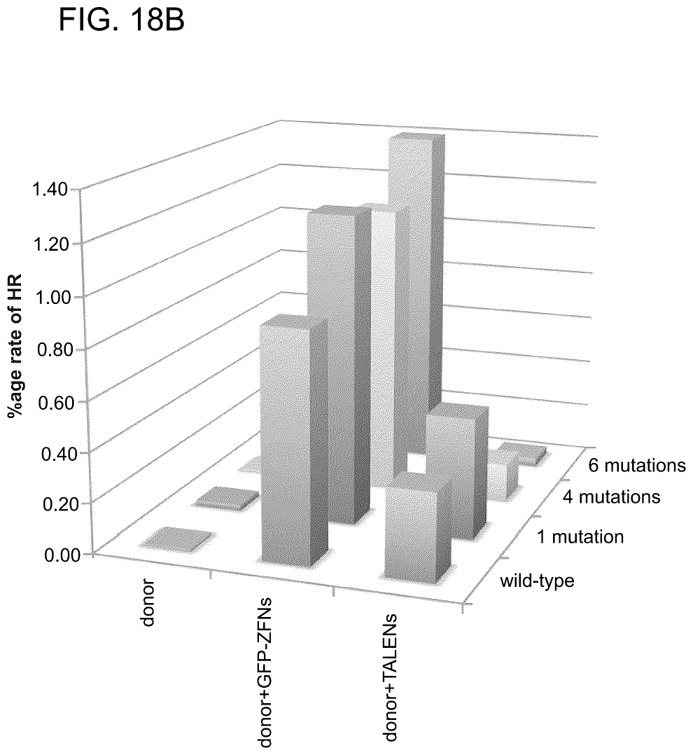

Using a nuclease mediated HR assay (FIGS. 18A-B) it was confirmed that 18-mer TALEs tolerate multiple mutations in their target sequences. As shown in FIGS. 18A-B certain mutations in the middle of the target lead to higher TALE activity, as determined via targeted experiments in a nuclease assay.

### EXAMPLE XII

### TALE Monomer Specificity Versus TALE Protein Specificity

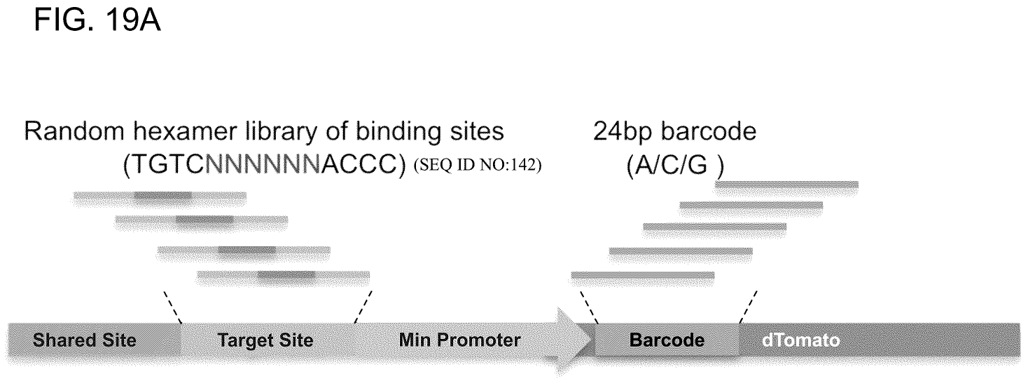

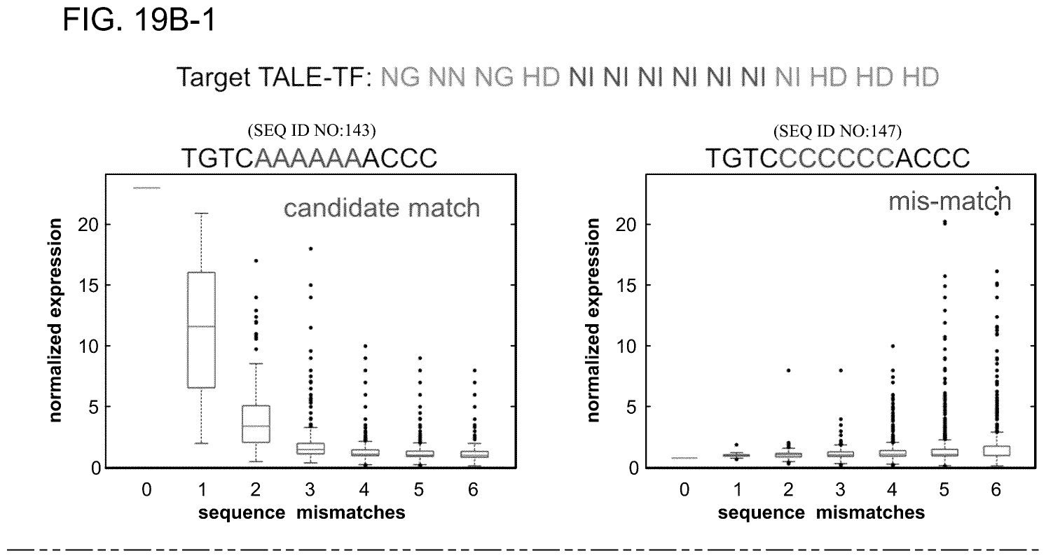

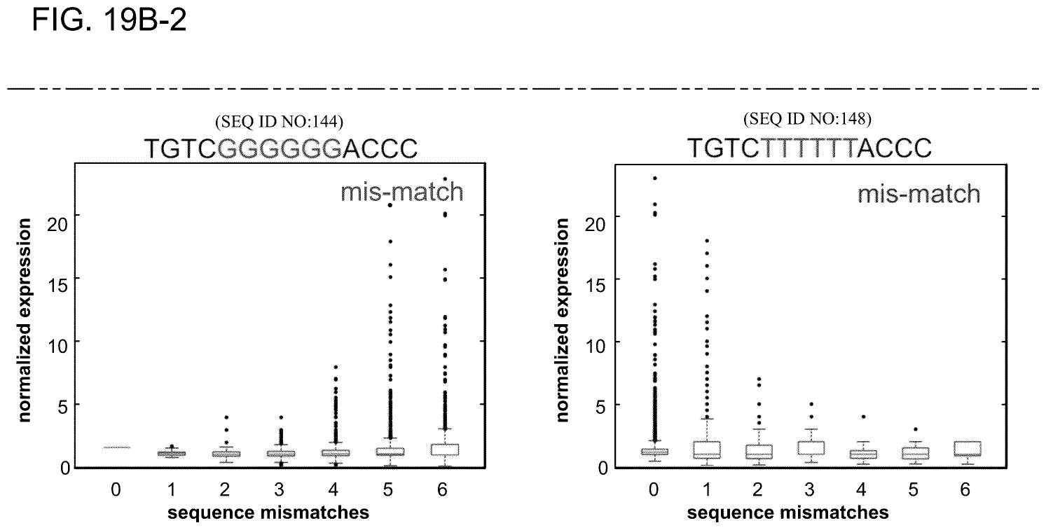

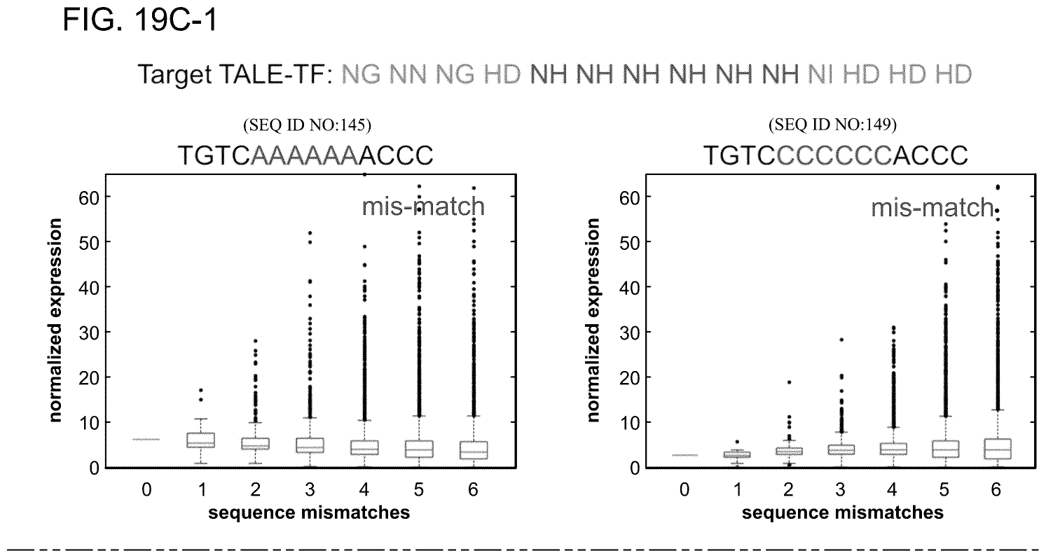

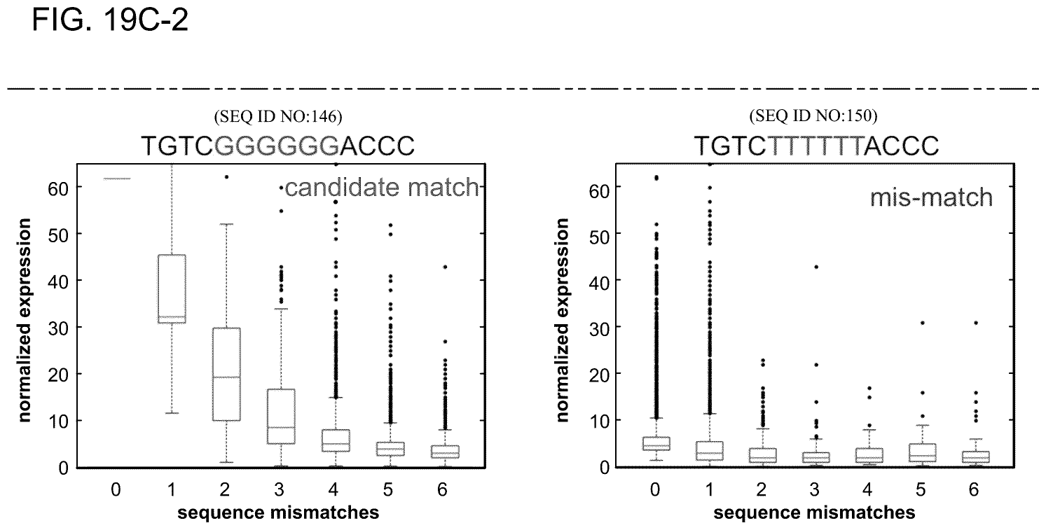

To decouple the role of individual repeat-variable diresidues (RVDs), it was confirmed that choice of RVDs did contribute to base specificity but TALE specificity is also a function of the binding energy of the protein as a whole. FIGS. 19A-19C-2 show a comparison of TALE monomer specificity versus TALE protein specificity. FIG. 19A: Using a modification of approach described in FIG. 2, the targeting landscape of 2 14-mer TALE-TFs bearing a contiguous set of 6 NI or 6 NH repeats was analyzed. In this approach, a reduced library of reporters bearing a degenerate 6-mer sequence in the middle was created and used to assay the TALE-TF specificity. FIGS. 19B-1-19C-2: In both instances, it was noted that the expected target sequence is enriched (i.e. one bearing 6 As for NI repeats, and 6 Gs for NH repeats). Each of these TALEs still tolerate 1-2 mismatches in the central 6-mer target sequence. While choice of monomers does contribute to base specificity, TALE specificity is also a function of the binding energy of the protein as a whole. According to one aspect, shorter engineered TALEs or TALEs bearing a composition of high and low affinity monomers result in higher specificity in genome engineering applications and FokI dimerization in nuclease applications allows for further reduction in off-target effects when using shorter TALEs.

### EXAMPLE XIII

### Off-set Nicking, Native Locus

FIG. 20A-B shows data related to off-set nicking. In the context of genome-editing, off-set nicks were created to generate DSBs. A large majority of nicks do not result in non-homologous end joining (NHEJ) mediated indels and thus when inducing off-set nicks, off-target single nick events will likely result in very low indel rates. Inducing off-set nicks to generate DSBs is effective at inducing gene disruption at both integrated reporter loci and at the native AAVSI genomic locus.

FIG. 20A: The native AAVSI locus with 8 gRNAs covering a 200 bp stretch of DNA was targeted: 4 targeting the sense strand (s1-4) and 4 the antisense strand (as1-4). Using the Cas9D10A mutant, which nicks the complementary strand, different two-way combinations of the gRNAs was used to induce a range of programmed 5′ or 3′ overhangs. FIG. 20B: Using a Sanger sequencing based assay, it was observed that while single gRNAs did not induce detectable NHEJ events, inducing off-set nicks to generate DSBs is highly effective at inducing gene disruption. Notably off-set nicks leading to 5′ overhangs result in more NHEJ events as opposed to 3′ overhangs. The number of Sanger sequencing clones is highlighted above the bars, and the predicted overhang lengths are indicated below the corresponding x-axis legends.

### EXAMPLE XIV

### Off-Set Nicking, NHEJ Profiles

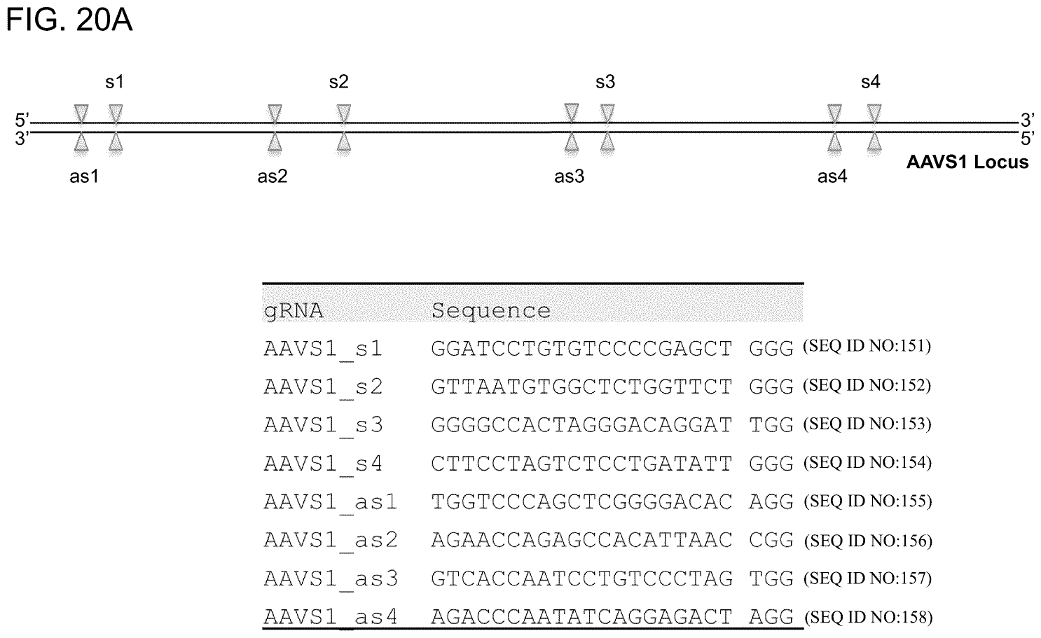

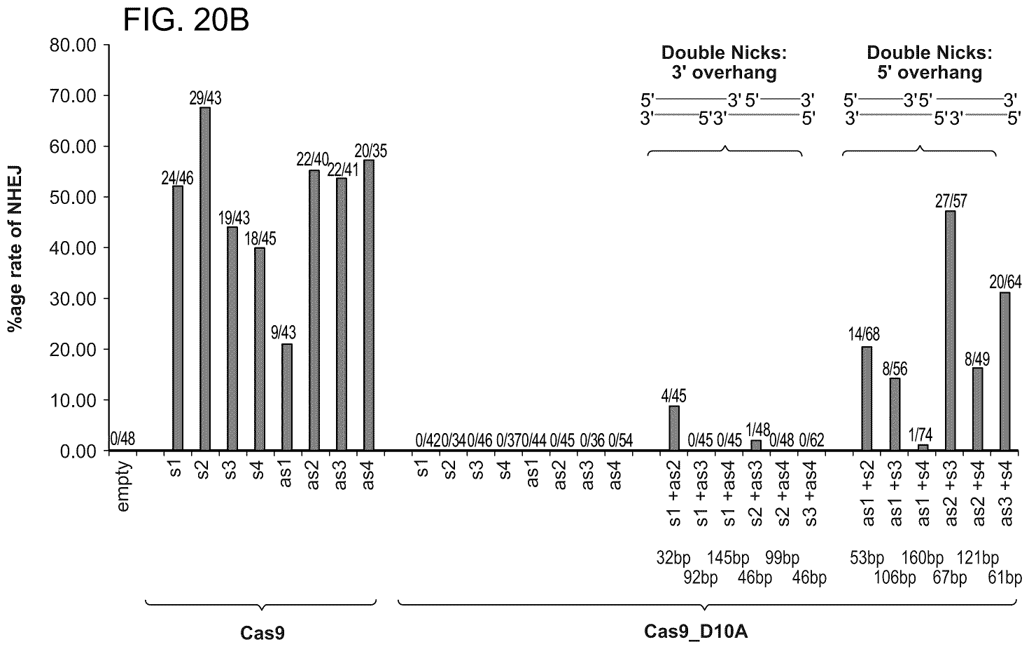

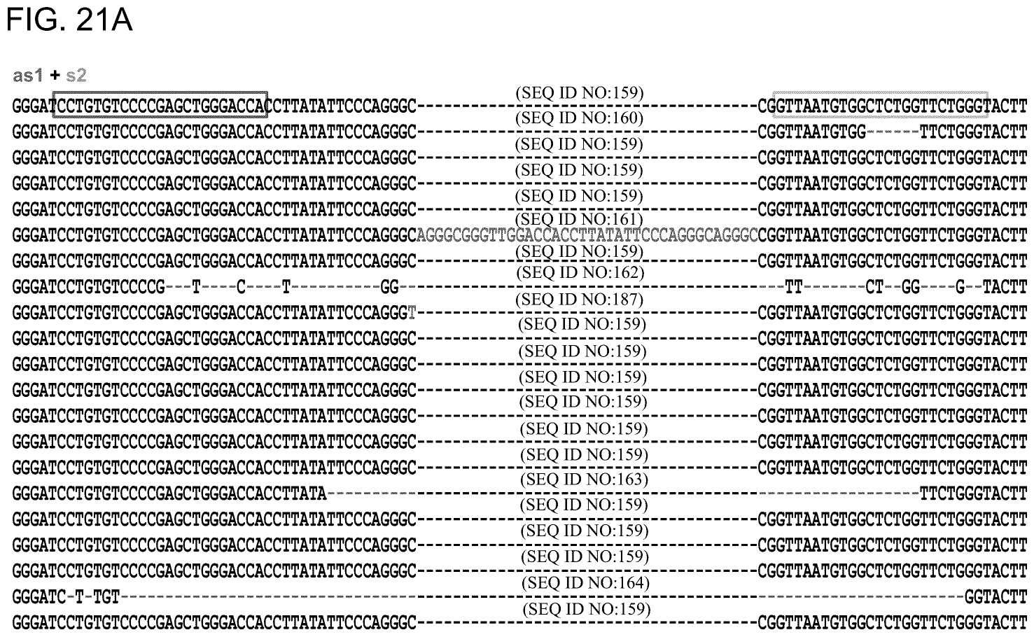

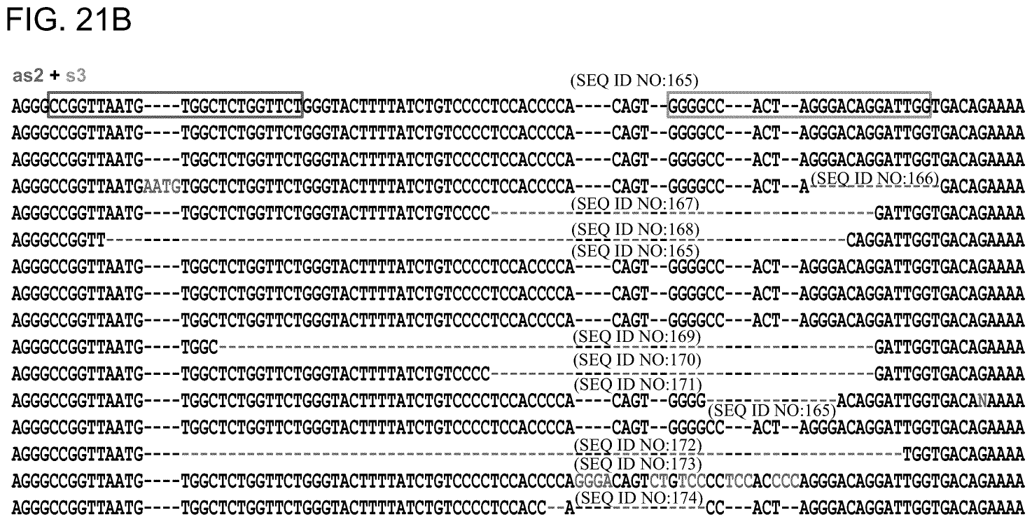

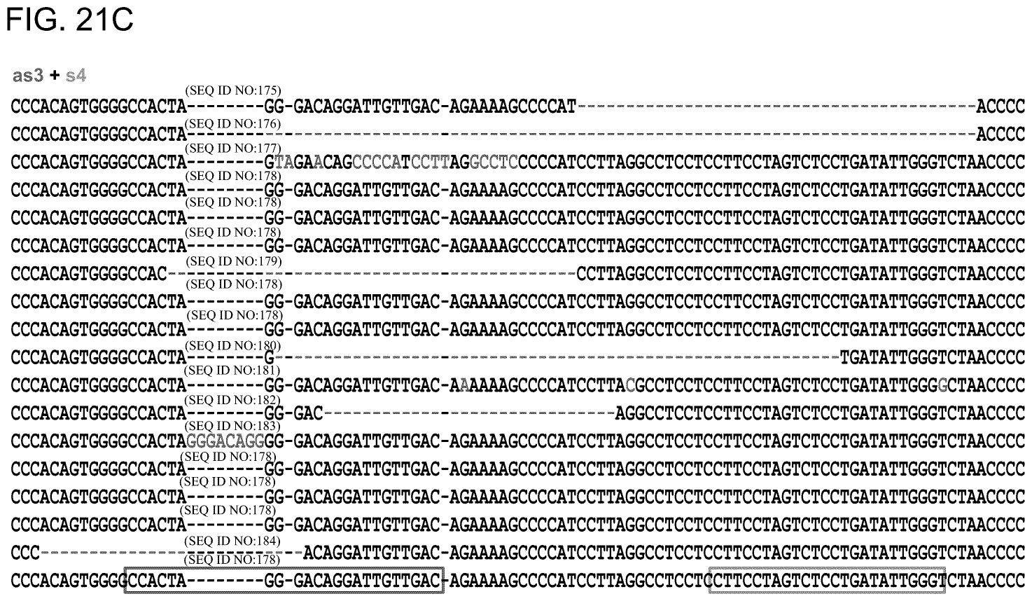

FIG. 21A-C is directed to off-set nicking and NHEJ profiles. Representative Sanger sequencing results of three different off-set nicking combinations is shown with positions of the targeting gRNAs highlighted by boxes. Furthermore, consistent with the standard model for homologous recombination (HR) mediated repair, engineering of 5′ overhangs via off-set nicks generated more robust NHEJ events than 3′ overhangs (FIG. 3B). In addition to a stimulation of NHEJ, robust induction of HR was observed when the 5′ overhangs were created. Generation of 3′ overhangs did not result in improvement of HR rates (FIG. 3C).

### EXAMPLE XV

TABLE 1
gRNA Targets for Endogenous Gene Regulation
Targets in the REX1, OCT4, SOX2 and NANOG
promoters used in Cas9-gRNA mediated activation
experiments are listed (SEQ ID NOs: 11-61).

| gRNA Name | gRNA Target |
|---|---|
| REX1 1 | ctggcggatcactcgcggtt agg |
| REX1 2 | cctcggcctccaaaagtgct agg |
| REX1 3 | acgctgattcctgcagatca ggg |
| REX1 4 | ccaggaatacgtatccacca ggg |
| REX1 5 | gccacacccaagcgatcaaa tgg |
| REX1 6 | aaataatacattctaaggta agg |
| REX1 7 | gctactggggaggctgaggc agg |
| REX1 8 | tagcaatacagtcacattaa tgg |
| REX1 9 | ctcatgtgatccccccgtct cgg |
| REX1 10 | ccgggcagagagtgaacgcg cgg |
| OCT4 1 | ttccttccctctcccgtgct tgg |
| OCT4 2 | tctctgcaaagcccctggag agg |
| OCT4 3 | aatgcagttgccgagtgcag tgg |
| OCT4 4 | cctcagcctcctaaagtgct ggg |
| OCT4 5 | gagtccaaatcctctttact agg |
| OCT4 6 | gagtgtctggatttgggata agg |
| OCT4 7 | cagcacctcatctcccagtg agg |
| OCT4 8 | tctaaaacccagggaatcat ggg |
| OCT4 9 | cacaaggcagccagggatcc agg |
| OCT4 10 | gatggcaagctgagaaacac tgg |
| OCT4 11 | tgaaatgcacgcatacaatt agg |
| OCT4 12 | ccagtccagacctggccttc tgg |
| OCT4 13 | cccagaaaaacagaccctga agg |
| OCT4 14 | aagggttgagcacttgttta ggg |
| OCT4 15 | atgtctgagttttggttgag agg |
| OCT4 16 | ggtcccttgaaggggaagta ggg |
| OCT4 17 | tggcagtctactcttgaaga tgg |
| OCT4 18 | ggcacagtgccagaggtctg tgg |
| OCT4 19 | taaaaataaaaaaactaaca ggg |
| OCT4 20 | tctgtgggggacctgcactg agg |
| OCT4 21 | ggccagaggtcaaggctagt ggg |
| SOX2 1 | cacgaccgaaacccttctta cgg |
| SOX2 2 | gttgaatgaagacagtctag tgg |
| SOX2 3 | taagaacagagcaagttacg tgg |
| SOX2 4 | tgtaaggtaagagaggagag cgg |
| SOX2 5 | tgacacaccaactcctgcac tgg |
| SOX2 6 | tttacccacttccttcgaaa agg |
| SOX2 7 | gtggctggcaggctggctct ggg |
| SOX2 8 | ctcccccggcctcccccgcg cgg |
| SOX2 9 | caaaacccggcagcgaggct ggg |
| SOX2 10 | aggagccgccgcgcgctgat tgg |
| NANOG 1 | cacacacacccacacgagat ggg |
| NANOG 2 | gaagaagctaaagagccaga ggg |
| NANOG 3 | atgagaatttcaataacctc agg |
| NANOG 4 | tcccgctctgttgcccaggc tgg |
| NANOG 5 | cagacacccaccaccatgct tgg |
| NANOG 6 | tcccaatttactgggattac agg |
| NANOG 7 | tgatttaaaagttggaaacg tgg |
| NANOG 8 | tctagttccccacctagtct ggg |
| NANOG 9 | gattaactgagaattcacaa ggg |
| NANOG 10 | cgccaggaggggtgggtcta agg |

### EXAMPLE XVI

### Table 2

### Summary of Statistical Analysis of Cas9-gRNA and TALE Specificity Data

Table 2(a) P-values for comparisons of normalized expression levels of TALE or Cas9-VP64 activators binding to target sequences with particular numbers of target site mutations. Normalized expression levels have been indicated by boxplots in the figures indicated in the Figure column, where the boxes represent the distributions of these levels by numbers of mismatches from the target site. P-values were computed using t-tests for each consecutive pair of numbers of mismatches in each boxplot, where the t-tests were either one sample or two sample t-tests (see Methods). Statistical significance was assessed using Bonferroni-corrected P-value thresholds, where the correction was based on the number of comparisons within each boxplot. Statistical significance symbols are: *** for P<0.0005/n, ** for P<0.005/n, * for P<0.05/n, and N.S. (Non-Significant) for P>=0.05/n, where n is the number of comparisons. Table 2(b) Statistical characterization of seed region in FIG. 2D: log10(P-values) indicating the degree of separation between expression values for Cas9N VP64+gRNA binding to target sequences with two mutations for those position pairs mutated within candidate seed regions at the 3′ end of the 20 bp target site vs. all other position pairs. The greatest separation, indicated by the largest -log10 (P-values) (highlighted above), is found in the last 8-9 bp of the target site. These positions may be interpreted as indicating the start of the “seed” region of this target site. See the section “Statistical characterization of seed region” in Methods for information on how the P-values were computed.

a

| | Expression level | | | | |
|---|---|---|---|---|---|
| | comparison: | | | | |
| Figure | mutations vs mutations | | t-test | P-value | Symbol |
| 2b | 0 | 1 | 1-samp | 7.8E−05 | ** |
| | 1 | 2 | 2-samp | 1.4E−06 | *** |
| | 2 | 3 | 2-samp | 4.0E−61 | *** |
| | 3 | 4 | 2-samp | 0 | *** |
| | 4 | 5 | 2-samp | 0 | *** |
| | 5 | 6 | 2-samp | 1.0E−217 | *** |
| | 6 | 7 | 2-samp | 1.7E−43 | *** |
| | 7 | 8 | 2-samp | 3.76−02 | N.S. |
| 2e | 0 | 1 | 1-samp | 8.9E−01 | N.S. |
| | 1 | 2 | 2-samp | 1.9E−06 | *** |
| | 2 | 3 | 2-samp | 5.0E−147 | *** |
| | 3 | 4 | 2-samp | 0 | *** |
| | 4 | 5 | 2-samp | 0 | *** |
| | 5 | 6 | 2-samp | 4.2E−62 | *** |
| | 6 | 7 | 2-samp | 1.6E−03 | * |
| | 7 | 8 | 2-samp | 4.7E−01 | N.S. |
| S7a | 0 | 1 | 1-samp | 5.2E−02 | N.S. |
| | 1 | 2 | 2-samp | 2.8E−05 | *** |
| | 2 | 3 | 2-samp | 3.5E−21 | *** |
| | 3 | 4 | 2-samp | 1.4E−58 | *** |
| | 4 | 5 | 2-samp | 8.3E−101 | *** |
| | 5 | 6 | 2-samp | 6.8E−94 | *** |
| | 6 | 7 | 2-samp | 1.8E−61 | *** |
| | 7 | 8 | 2-samp | 8.1E−24 | *** |
| S7d | 0 | 1 | 1-samp | 2.3E−18 | *** |
| and | 1 | 2 | 2-samp | 2.4E−08 | *** |
| S8d | 2 | 3 | 2-samp | 6.2E−54 | *** |
| | 3 | 4 | 2-samp | 4.0E−141 | *** |
| | 4 | 5 | 2-samp | 1.9E−20 | *** |
| | 5 | 6 | 2-samp | 1.2E−03 | * |
| | 6 | 7 | 2-samp | 3.8E−05 | *** |
| | 7 | 8 | 2-samp | 9.4E−01 | N.S. |
| S8c | 0 | 1 | 1-samp | 7.2E−03 | N.S. |
| | 1 | 2 | 2-samp | 5.0E−01 | N.S. |
| | 2 | 3 | 2-samp | 3.9E−84 | *** |
| | 3 | 4 | 2-samp | 8.5E−153 | *** |
| | 4 | 5 | 2-samp | 8.6E−76 | *** |
| | 5 | 6 | 2-samp | 1.6E−03 | * |
| | 6 | 7 | 2-samp | 7.1E−01 | N.S. |
| | 7 | 8 | 2-samp | 7.8E−02 | N.S. |
| S13a | 0 | 1 | 1-samp | 7.3E−01 | N.S. |
| (left) | 1 | 2 | 2-samp | 2.4E−06 | *** |
| | 2 | 3 | 2-samp | 7.2E−140 | *** |
| | 3 | 4 | 2-samp | 0 | *** |
| | 4 | 5 | 2-samp | 0 | *** |
| | 5 | 6 | 2-samp | 1.0E−72 | *** |
| | 6 | 7 | 2-samp | 4.0E−03 | * |
| S13a | 0 | 1 | 1-samp | 9.4E−02 | N.S. |
| (middle) | 1 | 2 | 2-samp | 5.2E−09 | *** |
| | 2 | 3 | 2-samp | 7.9E−86 | *** |
| | 3 | 4 | 2-samp | 2.9E−53 | *** |
| | 4 | 5 | 2-samp | 3.5E−10 | *** |
| S13a | 0 | 1 | 1-samp | 1.3E−13 | *** |
| (right) | 1 | 2 | 2-samp | 1.1E−04 | *** |
| | 2 | 3 | 2-samp | 3.7E−08 | *** |

b

| seed | Number | | |
|---|---|---|---|
| start | position pairs | | log10 |
| position | both in | not both | P-value |
| 2 | 171 | 19 | 3.11 |
| 3 | 153 | 37 | 1.46 |
| 4 | 136 | 54 | 2.01 |
| 5 | 120 | 70 | 3.34 |
| 6 | 105 | 85 | 5.65 |
| 7 | 91 | 99 | 7.34 |
| 8 | 78 | 112 | 6.61 |
| 9 | 66 | 124 | 7.1 |
| 10 | 55 | 135 | 9.72 |
| 11 | 45 | 145 | 9.83 |
| 12 | 36 | 154 | 10.44 |
| 13 | 28 | 162 | 10.72 |
| 14 | 21 | 169 | 8.97 |
| 15 | 15 | 175 | 5.61 |
| 16 | 10 | 180 | 3.34 |
| 17 | 6 | 184 | 2.26 |
| 18 | 3 | 187 | 1.16 |

### EXAMPLE XVII

### Sequences of Proteins and RNAs in the Examples

A. Sequences of the Cas9_{N-}VP64 activator constructs based on the m4 mutant are displayed below. Three versions were constructed with the Cas9ₘ₄^{VP64} and Cas9ₘ₄^{VP64}N fusion protein formats showing highest activity. Corresponding vectors for the m3 and m2 mutants (FIG. 4A) were also constructed (NLS and VP64 domains are highlighted).
Cas9ₘ₄^{VP64}

(SEQ ID NO: 2)
gccaccATGGACAAGAAGTACTCCATTGGGCTCGCTATCGGCACAAACAG
CGTCGGCTGGGCCGTCATTACGGACGAGTACAAGGTGCCGAGCAAAAAAT
TCAAAGTTCTGGGCAATACCGATCGCCACAGCATAAAGAAGAACCTCATT
GGCGCCCTCCTGTTCGACTCCGGGGAGACGGCCGAAGCCACGCGGCTCAA
AAGAACAGCACGGCGCAGATATACCCGCAGAAAGAATCGGATCTGCTACC
TGCAGGAGATCTTTAGTAATGAGATGGCTAAGGTGGATGACTCTTTCTTC
CATAGGCTGGAGGAGTCCTTTTTGGTGGAGGAGGATAAAAAGCACGAGCG
CCACCCAATCTTTGGCAATATCGTGGACGAGGTGGCGTACCATGAAAAGT
ACCCAACCATATATCATCTGAGGAAGAAGCTTGTAGACAGTACTGATAAG
GCTGACTTGCGGTTGATCTATCTCGCGCTGGCGCATATGATCAAATTTCG
GGGACACTTCCTCATCGAGGGGGACCTGAACCCAGACAACAGCGATGTCG
ACAAACTCTTTATCCAACTGGTTCAGACTTACAATCAGCTTTTCGAAGAG
AACCCGATCAACGCATCCGGAGTTGACGCCAAAGCAATCCTGAGCGCTAG
GCTGTCCAAATCCCGGCGGCTCGAAAACCTCATCGCACAGCTCCCTGGGG
AGAAGAAGAACGGCCTGTTTGGTAATCTTATCGCCCTGTCACTCGGGCTG
ACCCCCAACTTTAAATCTAACTTCGACCTGGCCGAAGATGCCAAGCTTCA
ACTGAGCAAAGACACCTACGATGATGATCTCGACAATCTGCTGGCCCAGA
TCGGCGACCAGTACGCAGACCTTTTTTTGGCGGCAAAGAACCTGTCAGAC
GCCATTCTGCTGAGTGATATTCTGCGAGTGAACACGGAGATCACCAAAGC
TCCGCTGAGCGCTAGTATGATCAAGCGCTATGATGAGCACCACCAAGACT
TGACTTTGCTGAAGGCCCTTGTCAGACAGCAACTGCCTGAGAAGTACAAG
GAAATTTTCTTCGATCAGTCTAAAAATGGCTACGCCGGATACATTGACGG
CGGAGCAAGCCAGGAGGAATTTTACAAATTTATTAAGCCCATCTTGGAAA
AAATGGACGGCACCGAGGAGCTGCTGGTAAAGCTTAACAGAGAAGATCTG
TTGCGCAAACAGCGCACTTTCGACAATGGAAGCATCCCCCACCAGATTCA
CCTGGGCGAACTGCACGCTATCCTCAGGCGGCAAGAGGATTTCTACCCCT
TTTTGAAAGATAACAGGGAAAAGATTGAGAAAATCCTCACATTTCGGATA
CCCTACTATGTAGGCCCCCTCGCCCGGGGAAATTCCAGATTCGCGTGGAT
GACTCGCAAATCAGAAGAGACCATCACTCCCTGGAACTTCGAGGAAGTCG
TGGATAAGGGGGCCTCTGCCCAGTCCTTCATCGAAAGGATGACTAACTTT
GATAAAAATCTGCCTAACGAAAAGGTGCTTCCTAAACACTCTCTGCTGTA
CGAGTACTTCACAGTTTATAACGAGCTCACCAAGGTCAAATACGTCACAG
AAGGGATGAGAAAGCCAGCATTCCTGTCTGGAGAGCAGAAGAAAGCTATC
GTGGACCTCCTCTTCAAGACGAACCGGAAAGTTACCGTGAAACAGCTCAA
AGAAGACTATTTCAAAAAGATTGAATGTTTCGACTCTGTTGAAATCAGCG
GAGTGGAGGATCGCTTCAACGCATCCCTGGGAACGTATCACGATCTCCTG
AAAATCATTAAAGACAAGGACTTCCTGGACAATGAGGAGAACGAGGACAT
TCTTGAGGACATTGTCCTCACCCTTACGTTGTTTGAAGATAGGGAGATGA
TTGAAGAACGCTTGAAAACTTACGCTCATCTCTTCGACGACAAAGTCATG
AAACAGCTCAAGAGGCGCCGATATACAGGATGGGGGCGGCTGTCAAGAAA
ACTGATCAATGGGATCCGAGACAAGCAGAGTGGAAAGACAATCCTGGATT
TTCTTAAGTCCGATGGATTTGCCAACCGGAACTTCATGCAGTTGATCCAT
GATGACTCTCTCACCTTTAAGGAGGACATCCAGAAAGCACAAGTTTCTGG
CCAGGGGGACAGTCTTCACGAGCACATCGCTAATCTTGCAGGTAGCCCAG
CTATCAAAAAGGGAATACTGCAGACCGTTAAGGTCGTGGATGAACTCGTC
AAAGTAATGGGAAGGCATAAGCCCGAGAATATCGTTATCGAGATGGCCCG
AGAGAACCAAACTACCCAGAAGGGACAGAAGAACAGTAGGGAAAGGATGA
AGAGGATTGAAGAGGGTATAAAAGAACTGGGGTCCCAAATCCTTAAGGAA
CACCCAGTTGAAAACACCCAGCTTCAGAATGAGAAGCTCTACCTGTACTA
CCTGCAGAACGGCAGGGACATGTACGTGGATCAGGAACTGGACATCAATC
GGCTCTCCGACTACGACGTGGCTGCTATCGTGCCCCAGTCTTTTCTCAAA
GATGATTCTATTGATAATAAAGTGTTGACAAGATCCGATAAAgcTAGAGG
GAAGAGTGATAACGTCCCCTCAGAAGAAGTTGTCAAGAAAATGAAAAATT
ATTGGCGGCAGCTGCTGAACGCCAAACTGATCACACAACGGAAGTTCGAT
AATCTGACTAAGGCTGAACGAGGTGGCCTGTCTGAGTTGGATAAAGCCGG
CTTCATCAAAAGGCAGCTTGTTGAGACACGCCAGATCACCAAGCACGTGG
CCCAAATTCTCGATTCACGCATGAACACCAAGTACGATGAAAATGACAAA
CTGATTCGAGAGGTGAAAGTTATTACTCTGAAGTCTAAGCTGGTCTCAGA
TTTCAGAAAGGACTTTCAGTTTTATAAGGTGAGAGAGATCAACAATTACC
ACCATGCGCATGATGCCTACCTGAATGCAGTGGTAGGCACTGCACTTATC
AAAAAATATCCCAAGCTTGAATCTGAATTTGTTTACGGAGACTATAAAGT
GTACGATGTTAGGAAAATGATCGCAAAGTCTGAGCAGGAAATAGGCAAGG
CCACCGCTAAGTACTTCTTTTACAGCAATATTATGAATTTTTTCAAGACC
GAGATTACACTGGCCAATGGAGAGATTCGGAAGCGACCACTTATCGAAAC
AAACGGAGAAACAGGAGAAATCGTGTGGGACAAGGGTAGGGATTTCGCGA
CAGTCCGGAAGGTCCTGTCCATGCCGCAGGTGAACATCGTTAAAAAGACC
GAAGTACAGACCGGAGGCTTCTCCAAGGAAAGTATCCTCCCGAAAAGGAA
CAGCGACAAGCTGATCGCACGCAAAAAAGATTGGGACCCCAAGAAATACG
GCGGATTCGATTCTCCTACAGTCGCTTACAGTGTACTGGTTGTGGCCAAA
GTGGAGAAAGGGAAGTCTAAAAAACTCAAAAGCGTCAAGGAACTGCTGGG
CATCACAATCATGGAGCGATCAAGCTTCGAAAAAAACCCCATCGACTTTC
TCGAGGCGAAAGGATATAAAGAGGTCAAAAAAGACCTCATCATTAAGCTT
CCCAAGTACTCTCTCTTTGAGCTTGAAAACGGCCGGAAACGAATGCTCGC
TAGTGCGGGCGAGCTGCAGAAAGGTAACGAGCTGGCACTGCCCTCTAAAT
ACGTTAATTTCTTGTATCTGGCCAGCCACTATGAAAAGCTCAAAGGGTCT
CCCGAAGATAATGAGCAGAAGCAGCTGTTCGTGGAACAACACAAACACTA
CCTTGATGAGATCATCGAGCAAATAAGCGAATTCTCCAAAAGAGTGATCC
TCGCCGACGCTAACCTCGATAAGGTGCTTTCTGCTTACAATAAGCACAGG
GATAAGCCCATCAGGGAGCAGGCAGAAAACATTATCCACTTGTTTACTCT
GACCAACTTGGGCGCGCCTGCAGCCTTCAAGTACTTCGACACCACCATAG
ACAGAAAGCGGTACACCTCTACAAAGGAGGTCCTGGACGCCACACTGATT
CATCAGTCAATTACGGGGCTCTATGAAACAAGAATCGACCTCTCTCAGCT
CGGTGGAGACAGCAGGGCTGACCCCAAGAAGAAGAGGAAGGTGGAGGCCA
GCGGTTCCGGACGGGCTGACGCATTGGACGATTTTGATCTGGATATGCTG
GGAAGTGACGCCCTCGATGATTTTGACCTTGACATGCTTGGTTCGGATGC
CCTTGATGACTTTGACCTCGACATGCTCGGCAGTGACGCCCTTGATGATT
CGACCTGGACATGCTGATTAACTCTAGATGA

Cas9ₘ₄^{VP64}N Sequences

(SEQ ID NO: 3)
gccaccATGCCCAAGAAGAAGAGGAAGGTGGGAAGGGGGATGGACAAGAA
GTACTCCATTGGGCTCGCTATCGGCACAAACAGCGTCGGCTGGGCCGTCA
TTACGGACGAGTACAAGGTGCCGAGCAAAAAATTCAAAGTTCTGGGCAAT
ACCGATCGCCACAGCATAAAGAAGAACCTCATTGGCGCCCTCCTGTTCGA
CTCCGGGGAGACGGCCGAAGCCACGCGGCTCAAAAGAACAGCACGGCGCA
GATATACCCGCAGAAAGAATCGGATCTGCTACCTGCAGGAGATCTTTAGT
AATGAGATGGCTAAGGTGGATGACTCTTTCTTCCATAGGCTGGAGGAGTC
CTTTTTGGTGGAGGAGGATAAAAAGCACGAGCGCCACCCAATCTTTGGCA
ATATCGTGGACGAGGTGGCGTACCATGAAAAGTACCCAACCATATATCAT
CTGAGGAAGAAGCTTGTAGACAGTACTGATAAGGCTGACTTGCGGTTGAT
CTATCTCGCGCTGGCGCATATGATCAAATTTCGGGGACACTTCCTCATCG
AGGGGGACCTGAACCCAGACAACAGCGATGTCGACAAACTCTTTATCCAA
CTGGTTCAGACTTACAATCAGCTTTTCGAAGAGAACCCGATCAACGCATC
CGGAGTTGACGCCAAAGCAATCCTGAGCGCTAGGCTGTCCAAATCCCGGC
GGCTCGAAAACCTCATCGCACAGCTCCCTGGGGAGAAGAAGAACGGCCTG
TTTGGTAATCTTATCGCCCTGTCACTCGGGCTGACCCCCAACTTTAAATC
TAACTTCGACCTGGCCGAAGATGCCAAGCTTCAACTGAGCAAAGACACCT
ACGATGATGATCTCGACAATCTGCTGGCCCAGATCGGCGACCAGTACGCA
GACCTTTTTTTGGCGGCAAAGAACCTGTCAGACGCCATTCTGCTGAGTGA
TATTCTGCGAGTGAACACGGAGATCACCAAAGCTCCGCTGAGCGCTAGTA
TGATCAAGCGCTATGATGAGCACCACCAAGACTTGACTTTGCTGAAGGCC
CTTGTCAGACAGCAACTGCCTGAGAAGTACAAGGAAATTTTCTTCGATCA
GTCTAAAAATGGCTACGCCGGATACATTGACGGCGGAGCAAGCCAGGAGG
AATTTTACAAATTTATTAAGCCCATCTTGGAAAAAATGGACGGCACCGAG
GAGCTGCTGGTAAAGCTTAACAGAGAAGATCTGTTGCGCAAACAGCGCAC
TTTCGACAATGGAAGCATCCCCCACCAGATTCACCTGGGCGAACTGCACG
CTATCCTCAGGCGGCAAGAGGATTTCTACCCCTTTTTGAAAGATAACAGG
GAAAAGATTGAGAAAATCCTCACATTTCGGATACCCTACTATGTAGGCCC
CCTCGCCCGGGGAAATTCCAGATTCGCGTGGATGACTCGCAAATCAGAAG
AGACCATCACTCCCTGGAACTTCGAGGAAGTCGTGGATAAGGGGGCCTCT
GCCCAGTCCTTCATCGAAAGGATGACTAACTTTGATAAAAATCTGCCTAA
CGAAAAGGTGCTTCCTAAACACTCTCTGCTGTACGAGTACTTCACAGTTT
ATAACGAGCTCACCAAGGTCAAATACGTCACAGAAGGGATGAGAAAGCCA
GCATTCCTGTCTGGAGAGCAGAAGAAAGCTATCGTGGACCTCCTCTTCAA
GACGAACCGGAAAGTTACCGTGAAACAGCTCAAAGAAGACTATTTCAAAA
AGATTGAATGTTTCGACTCTGTTGAAATCAGCGGAGTGGAGGATCGCTTC
AACGCATCCCTGGGAACGTATCACGATCTCCTGAAAATCATTAAAGACAA
GGACTTCCTGGACAATGAGGAGAACGAGGACATTCTTGAGGACATTGTCC
TCACCCTTACGTTGTTTGAAGATAGGGAGATGATTGAAGAACGCTTGAAA
ACTTACGCTCATCTCTTCGACGACAAAGTCATGAAACAGCTCAAGAGGCG
CCGATATACAGGATGGGGGCGGCTGTCAAGAAAACTGATCAATGGGATCC
GAGACAAGCAGAGTGGAAAGACAATCCTGGATTTTCTTAAGTCCGATGGA
TTTGCCAACCGGAACTTCATGCAGTTGATCCATGATGACTCTCTCACCTT
TAAGGAGGACATCCAGAAAGCACAAGTTTCTGGCCAGGGGGACAGTCTTC
ACGAGCACATCGCTAATCTTGCAGGTAGCCCAGCTATCAAAAAGGGAATA
CTGCAGACCGTTAAGGTCGTGGATGAACTCGTCAAAGTAATGGGAAGGCA
TAAGCCCGAGAATATCGTTATCGAGATGGCCCGAGAGAACCAAACTACCC
AGAAGGGACAGAAGAACAGTAGGGAAAGGATGAAGAGGATTGAAGAGGGT
ATAAAAGAACTGGGGTCCCAAATCCTTAAGGAACACCCAGTTGAAAACAC
CCAGCTTCAGAATGAGAAGCTCTACCTGTACTACCTGCAGAACGGCAGGG
ACATGTACGTGGATCAGGAACTGGACATCAATCGGCTCTCCGACTACGAC
GTGGCTGCTATCGTGCCCCAGTCTTTTCTCAAAGATGATTCTATTGATAA
TAAAGTGTTGACAAGATCCGATAAAgcTAGAGGGAAGAGTGATAACGTCC
CCTCAGAAGAAGTTGTCAAGAAAATGAAAAATTATTGGCGGCAGCTGCTG
AACGCCAAACTGATCACACAACGGAAGTTCGATAATCTGACTAAGGCTGA
ACGAGGTGGCCTGTCTGAGTTGGATAAAGCCGGCTTCATCAAAAGGCAGC
TTGTTGAGACACGCCAGATCACCAAGCACGTGGCCCAAATTCTCGATTCA
CGCATGAACACCAAGTACGATGAAAATGACAAACTGATTCGAGAGGTGAA
AGTTATTACTCTGAAGTCTAAGCTGGTCTCAGATTTCAGAAAGGACTTTC
AGTTTTATAAGGTGAGAGAGATCAACAATTACCACCATGCGCATGATGCC
TACCTGAATGCAGTGGTAGGCACTGCACTTATCAAAAAATATCCCAAGCT
TGAATCTGAATTTGTTTACGGAGACTATAAAGTGTACGATGTTAGGAAAA
TGATCGCAAAGTCTGAGCAGGAAATAGGCAAGGCCACCGCTAAGTACTTC
TTTTACAGCAATATTATGAATTTTTTCAAGACCGAGATTACACTGGCCAA
TGGAGAGATTCGGAAGCGACCACTTATCGAAACAAACGGAGAAACAGGAG
AAATCGTGTGGGACAAGGGTAGGGATTTCGCGACAGTCCGGAAGGTCCTG
TCCATGCCGCAGGTGAACATCGTTAAAAAGACCGAAGTACAGACCGGAGG
CTTCTCCAAGGAAAGTATCCTCCCGAAAAGGAACAGCGACAAGCTGATCG
CACGCAAAAAAGATTGGGACCCCAAGAAATACGGCGGATTCGATTCTCCT
ACAGTCGCTTACAGTGTACTGGTTGTGGCCAAAGTGGAGAAAGGGAAGTC
TAAAAAACTCAAAAGCGTCAAGGAACTGCTGGGCATCACAATCATGGAGC
GATCAAGCTTCGAAAAAAACCCCATCGACTTTCTCGAGGCGAAAGGATAT
AAAGAGGTCAAAAAAGACCTCATCATTAAGCTTCCCAAGTACTCTCTCTT
TGAGCTTGAAAACGGCCGGAAACGAATGCTCGCTAGTGCGGGCGAGCTGC
AGAAAGGTAACGAGCTGGCACTGCCCTCTAAATACGTTAATTTCTTGTAT
CTGGCCAGCCACTATGAAAAGCTCAAAGGGTCTCCCGAAGATAATGAGCA
GAAGCAGCTGTTCGTGGAACAACACAAACACTACCTTGATGAGATCATCG
AGCAAATAAGCGAATTCTCCAAAAGAGTGATCCTCGCCGACGCTAACCTC
GATAAGGTGCTTTCTGCTTACAATAAGCACAGGGATAAGCCCATCAGGGA
GCAGGCAGAAAACATTATCCACTTGTTTACTCTGACCAACTTGGGCGCGC
CTGCAGCCTTCAAGTACTTCGACACCACCATAGACAGAAAGCGGTACACC
TCTACAAAGGAGGTCCTGGACGCCACACTGATTCATCAGTCAATTACGGG
GCTCTATGAAACAAGAATCGACCTCTCTCAGCTCGGTGGAGACAGCAGGG
CTGACCCCAAGAAGAAGAGGAAGGTGGAGGCCAGCGGTTCCGGACGGGCT
GACGCATTGGACGATTTTGATCTGGATATGCTGGGAAGTGACGCCCTCGA
TGATTTTGACCTTGACATGCTTGGTTCGGATGCCCTTGATGACTTTCAGG
TCGACATGCTCGGCAGTGACGCCCTTGATCATTTCGACCTGGACATGCTG
ATTAACTCTAGATGA

(SEQ ID NO: 4)
gccaccATGGACAAGAAGTACTCCATTGGGCTCGCTATCGGCACAAACAG
CGTCGGCTGGGCCGTCATTACGGACGAGTACAAGGTGCCGAGCAAAAAAT
TCAAAGTTCTGGGCAATACCGATCGCCACAGCATAAAGAAGAACCTCATT
GGCGCCCTCCTGTTCGACTCCGGGGAGACGGCCGAAGCCACGCGGCTCAA
AAGAACAGCACGGCGCAGATATACCCGCAGAAAGAATCGGATCTGCTACC
TGCAGGAGATCTTTAGTAATGAGATGGCTAAGGTGGATGACTCTTTCTTC
CATAGGCTGGAGGAGTCCTTTTTGGTGGAGGAGGATAAAAAGCACGAGCG
CCACCCAATCTTTGGCAATATCGTGGACGAGGTGGCGTACCATGAAAAGT
ACCCAACCATATATCATCTGAGGAAGAAGCTTGTAGACAGTACTGATAAG
GCTGACTTGCGGTTGATCTATCTCGCGCTGGCGCATATGATCAAATTTCG
GGGACACTTCCTCATCGAGGGGGACCTGAACCCAGACAACAGCGATGTCG
ACAAACTCTTTATCCAACTGGTTCAGACTTACAATCAGCTTTTCGAAGAG
AACCCGATCAACGCATCCGGAGTTGACGCCAAAGCAATCCTGAGCGCTAG
GCTGTCCAAATCCCGGCGGCTCGAAAACCTCATCGCACAGCTCCCTGGGG
AGAAGAAGAACGGCCTGTTTGGTAATCTTATCGCCCTGTCACTCGGGCTG
ACCCCCAACTTTAAATCTAACTTCGACCTGGCCGAAGATGCCAAGCTTCA
ACTGAGCAAAGACACCTACGATGATGATCTCGACAATCTGCTGGCCCAGA
TCGGCGACCAGTACGCAGACCTTTTTTTGGCGGCAAAGAACCTGTCAGAC
GCCATTCTGCTGAGTGATATTCTGCGAGTGAACACGGAGATCACCAAAGC
TCCGCTGAGCGCTAGTATGATCAAGCGCTATGATGAGCACCACCAAGACT
TGACTTTGCTGAAGGCCCTTGTCAGACAGCAACTGCCTGAGAAGTACAAG
GAAATTTTCTTCGATCAGTCTAAAAATGGCTACGCCGGATACATTGACGG
CGGAGCAAGCCAGGAGGAATTTTACAAATTTATTAAGCCCATCTTGGAAA
AAATGGACGGCACCGAGGAGCTGCTGGTAAAGCTTAACAGAGAAGATCTG
TTGCGCAAACAGCGCACTTTCGACAATGGAAGCATCCCCCACCAGATTCA
CCTGGGCGAACTGCACGCTATCCTCAGGCGGCAAGAGGATTTCTACCCCT
TTTTGAAAGATAACAGGGAAAAGATTGAGAAAATCCTCACATTTCGGATA
CCCTACTATGTAGGCCCCCTCGCCCGGGGAAATTCCAGATTCGCGTGGAT
GACTCGCAAATCAGAAGAGACCATCACTCCCTGGAACTTCGAGGAAGTCG
TGGATAAGGGGGCCTCTGCCCAGTCCTTCATCGAAAGGATGACTAACTTT
GATAAAAATCTGCCTAACGAAAAGGTGCTTCCTAAACACTCTCTGCTGTA
CGAGTACTTCACAGTTTATAACGAGCTCACCAAGGTCAAATACGTCACAG
AAGGGATGAGAAAGCCAGCATTCCTGTCTGGAGAGCAGAAGAAAGCTATC
GTGGACCTCCTCTTCAAGACGAACCGGAAAGTTACCGTGAAACAGCTCAA
AGAAGACTATTTCAAAAAGATTGAATGTTTCGACTCTGTTGAAATCAGCG
GAGTGGAGGATCGCTTCAACGCATCCCTGGGAACGTATCACGATCTCCTG
AAAATCATTAAAGACAAGGACTTCCTGGACAATGAGGAGAACGAGGACAT
TCTTGAGGACATTGTCCTCACCCTTACGTTGTTTGAAGATAGGGAGATGA
TTGAAGAACGCTTGAAAACTTACGCTCATCTCTTCGACGACAAAGTCATG
AAACAGCTCAAGAGGCGCCGATATACAGGATGGGGGCGGCTGTCAAGAAA
ACTGATCAATGGGATCCGAGACAAGCAGAGTGGAAAGACAATCCTGGATT
TTCTTAAGTCCGATGGATTTGCCAACCGGAACTTCATGCAGTTGATCCAT
GATGACTCTCTCACCTTTAAGGAGGACATCCAGAAAGCACAAGTTTCTGG
CCAGGGGGACAGTCTTCACGAGCACATCGCTAATCTTGCAGGTAGCCCAG
CTATCAAAAAGGGAATACTGCAGACCGTTAAGGTCGTGGATGAACTCGTC
AAAGTAATGGGAAGGCATAAGCCCGAGAATATCGTTATCGAGATGGCCCG
AGAGAACCAAACTACCCAGAAGGGACAGAAGAACAGTAGGGAAAGGATGA
AGAGGATTGAAGAGGGTATAAAAGAACTGGGGTCCCAAATCCTTAAGGAA
CACCCAGTTGAAAACACCCAGCTTCAGAATGAGAAGCTCTACCTGTACTA
CCTGCAGAACGGCAGGGACATGTACGTGGATCAGGAACTGGACATCAATC
GGCTCTCCGACTACGACGTGGCTGCTATCGTGCCCCAGTCTTTTCTCAAA
GATGATTCTATTGATAATAAAGTGTTGACAAGATCCGATAAAgcTAGAGG
GAAGAGTGATAACGTCCCCTCAGAAGAAGTTGTCAAGAAAATGAAAAATT
ATTGGCGGCAGCTGCTGAACGCCAAACTGATCACACAACGGAAGTTCGAT
AATCTGACTAAGGCTGAACGAGGTGGCCTGTCTGAGTTGGATAAAGCCGG
CTTCATCAAAAGGCAGCTTGTTGAGACACGCCAGATCACCAAGCACGTGG
CCCAAATTCTCGATTCACGCATGAACACCAAGTACGATGAAAATGACAAA
CTGATTCGAGAGGTGAAAGTTATTACTCTGAAGTCTAAGCTGGTCTCAGA
TTTCAGAAAGGACTTTCAGTTTTATAAGGTGAGAGAGATCAACAATTACC
ACCATGCGCATGATGCCTACCTGAATGCAGTGGTAGGCACTGCACTTATC
AAAAAATATCCCAAGCTTGAATCTGAATTTGTTTACGGAGACTATAAAGT
GTACGATGTTAGGAAAATGATCGCAAAGTCTGAGCAGGAAATAGGCAAGG
CCACCGCTAAGTACTTCTTTTACAGCAATATTATGAATTTTTTCAAGACC
GAGATTACACTGGCCAATGGAGAGATTCGGAAGCGACCACTTATCGAAAC
AAACGGAGAAACAGGAGAAATCGTGTGGGACAAGGGTAGGGATTTCGCGA
CAGTCCGGAAGGTCCTGTCCATGCCGCAGGTGAACATCGTTAAAAAGACC
GAAGTACAGACCGGAGGCTTCTCCAAGGAAAGTATCCTCCCGAAAAGGAA
CAGCGACAAGCTGATCGCACGCAAAAAAGATTGGGACCCCAAGAAATACG
GCGGATTCGATTCTCCTACAGTCGCTTACAGTGTACTGGTTGTGGCCAAA
GTGGAGAAAGGGAAGTCTAAAAAACTCAAAAGCGTCAAGGAACTGCTGGG
CATCACAATCATGGAGCGATCAAGCTTCGAAAAAAACCCCATCGACTTTC
TCGAGGCGAAAGGATATAAAGAGGTCAAAAAAGACCTCATCATTAAGCTT
CCCAAGTACTCTCTCTTTGAGCTTGAAAACGGCCGGAAACGAATGCTCGC
TAGTGCGGGCGAGCTGCAGAAAGGTAACGAGCTGGCACTGCCCTCTAAAT
ACGTTAATTTCTTGTATCTGGCCAGCCACTATGAAAAGCTCAAAGGGTCT
CCCGAAGATAATGAGCAGAAGCAGCTGTTCGTGGAACAACACAAACACTA
CCTTGATGAGATCATCGAGCAAATAAGCGAATTCTCCAAAAGAGTGATCC
TCGCCGACGCTAACCTCGATAAGGTGCTTTCTGCTTACAATAAGCACAGG
GATAAGCCCATCAGGGAGCAGGCAGAAAACATTATCCACTTGTTTACTCT
GACCAACTTGGGCGCGCCTGCAGCCTTCAAGTACTTCGACACCACCATAG
ACAGAAAGCGGTACACCTCTACAAAGGAGGTCCTGGACGCCACACTGATT
CATCAGTCAATTACGGGGCTCTATGAAACAAGAATCGACCTCTCTCAGCT
CGGTGGAGACAGCAGGGCTGACCCCAAGAAGAAGAGGAAGGTGGAGGCCA
GCGGTTCCGGACGGGCTGACGCATTGGACGATTTTGATCTGGATATGCTG
GGAAGTGACGCCCTCGATGATTTTGACCTTGACATGCTTGGTCTGGACAT
GCTGATTAACTCTAGAGCGGCCGCAGATCCAAAAAAGAAGAGAAAGGTAG
ATCCAAAAAAGAAGAGAAAGGTAGATCCAAAAAAGAAGAGAAAGGTAGAT
ACGGCCGCATAG

B. Sequences of the MS2-activator constructs and corresponding gRNA backbone vector with 2X MS2 aptamer domains is provided below (NLS, VP64, gRNA spacer, and MS2-binding RNA stem loop domains are highlighted). Two versions of the former were constructed with the MS2_{VP64}N fusion protein format showing highest activity.

### MS2_{VP64}N

(SEQ ID NO: 5)
gccaccATGGGACCTAAGAAAAAGAGGAAGGTGGCGGCCGCTTCTAGAAT
GGCTTCTAACTTTACTCAGTTCGTTCTCGTCGACAATGGCGGAACTGGCG
ACGTGACTGTCGCCCCAAGCAACTTCGCTAACGGGATCGCTGAATGGATC
AGCTCTAACTCGCGTTCACAGGCTTACAAAGTAACCTGTAGCGTTCGTCA
GAGCTCTGCGCAGAATCGCAAATACACCATCAAAGTCGAGGTGCCTAAAG
GCGCCTGGCGTTCGTACTTAAATATGGAACTAACCATTCCAATTTTCGCC
ACGAATTCCGACTGCGAGCTTATTGTTAAGGCAATGCAAGGTCTCCTAAA
AGATGGAAACCCGATTCCCTCAGCAATCGCAGCAAACTCCGGCATCTACG
AGGCCAGCGGTTCCGGACGGGCTGACGCATTGGACGATTTTGATCTGGAT
ATGCTGGGAAGTGACGCCCTCGATGATTTTGACCTTGACATGCTTGGTCG
GATGCCCTTGATGACTTTGACCTCGACATGCTCGGCAGTGACGCCCTTGA
TCATTTCGACCTGGACATGCTGATTAACTCTAGATGA

### MS2_{VP64}C

(SEQ ID NO: 6)
gccaccATGGGACCTAAGAAAAAGAGGAAGGTGGCGGCCGCTTCTAGAAT
GGCTTCTAACTTTACTCAGTTCGTTCTCGTCGACAATGGCGGAACTGGCG
ACGTGACTGTCGCCCCAAGCAACTTCGCTAACGGGATCGCTGAATGGATC
AGCTCTAACTCGCGTTCACAGGCTTACAAAGTAACCTGTAGCGTTCGTCA
GAGCTCTGCGCAGAATCGCAAATACACCATCAAAGTCGAGGTGCCTAAAG
GCGCCTGGCGTTCGTACTTAAATATGGAACTAACCATTCCAATTTTCGCC
ACGAATTCCGACTGCGAGCTTATTGTTAAGGCAATGCAAGGTCTCCTAAA
AGATGGAAACCCGATTCCCTCAGCAATCGCAGCAAACTCCGGCATCTACG
AGGCCAGCGGTTCCGGACGGGCTGACGCATTGGACGATTTTGATCTGGAT
ATGCTGGGAAGTGACGCCCTCGATCATTTTGACCTTGACATGCTTGGTTC
GGATGCCCTTGATGACTTGACCTCGACATGCTCGGCAGTGACGCCCTTGA
TGATTTCGACCTGGACATGCTGATTAACTCTAGAGCGGCCGCAGATCCAA
AAAAGAAGAGAAAGGTAGATCCAAAAAAGAAGAGAAAGGTAGATCCAAAA
AAGAAGAGAAAGGTAGATACGGCCGCATAG

gRNA_{2XMS2}

(SEQ ID NO: 7)
TGTACAAAAAAGCAGGCTTTAAAGGAACCAATTCAGTCGACTGGATCCGG
TACCAAGGTCGGGCAGGAAGAGGGCCTATTTCCCATGATTCCTTCATATT
TGCATATACGATACAAGGCTGTTAGAGAGATAATTAGAATTAATTTGACT
GTAAACACAAAGATATTAGTACAAAATACGTGACGTAGAAAGTAATAATT
TCTTGGGTAGTTTGCAGTTTTAAAATTATGTTTTAAAATGGACTATCATA
TGCTTACCGTAACTTGAAAGTATTTCGATTTCTTGGCTTTATATATCTTG
TGGAAAGGACGAAACACCGNNNNNNNNNNNNNNNNNNNNGTTTTAGAGCT
AGAAATAGCAAGTTAAAATAAGGCTAGTCCGTTATCAACTTGAAAAAGTG
GCACCGAGTCGGTGCTCTGCAGGTCGACTCTAGAAAACATGAGGATCACC
CATGTCTGCAGTATTCCCGGGTTCATTAGATCCTAAGGTACCTAATTGCC
TAGAAAACATGAGGATCACCCATGTCTGCAGGTCGACTCTAGAAATTTTT
TCTAGAC

C. dTomato fluorescence based transcriptional activation reporter sequences are listed below (ISceI control-TF target. gRNA targets, minCMV promoter and FLAG tag+dTomato sequences are highlighted).

### >TF Reporter 1

(SEQ ID NO: 8)
TAGGGATAACAGGTAATAGTGTCCCCTCCACCCCACAGTGGGGCGAGGTA
GGCGTGTACGGTGGGAGGCCTATATAAGCAGAGCTCGTTTAGTGAACCGT
CAGATCGCCTGGAGAATTCgccaccatgGACTACAAGGATGACGACGATA
AAACTTCCGGTGGCGGACTGGGTTCCACCGTGAGCAAGGGCGAGGAGGTC
ATCAAAGAGTTCATGCGCTTCAAGGTGCGCATGGAGGGCTCCATGAACGG
CCACGAGTTCGAGATCGAGGGCGAGGGCCGCCCCTACGAGGGCACCCAGA
CCGCCAAGCTGAAGGTGACCAAGGGCGGCCCCCTGCCCTTCGCCTGGGAC
ATCCTGTCCCCCCAGTTCATGTACGGCTCCAAGGCGTACGTGAAGCACCC
CGCCGACATCCCCGATTACAAGAAGCTGTCCTTCCCCGAGGGCTTCAAGT
GGGAGCGCGTGATGAACTTCGAGGACGGCGGTCTGGTGACCGTGACCCAG
GACTCCTCCCTGCAGGACGGCACGCTGATCTACAAGGTGAAGATGCGCGG
CACCAACTTCCCCCCGACGGCCCCGTAATGCAGAAGAAGACCAGGGCTGG
GAGGCCTCCACCGAGCGCCTGTACCCCCGCGCGGCGTGCTGAAGGGCGAG
ATCCACCAGGCCCTGAAGCTGAAGGACGGCGGCCACTACCTGGTGGAGTT
CAAGACCATCTACATGGCCAAGAAGCCCGTGCAACTGCCCGGCTACTACT
ACGTGGACACCAAGCTGGACATCACCTCCCACAACGAGGACTACACCATC
GTGGAACAGTACGAGCGCTCCGAGGGCCGCCACCACCTGTTCCTGTACGG
CATGGACGAGCTGTACAAGTAA

### TF Reporter 2

(SEQ ID NO: 9)
TAGGGATAACAGGGTAATAGTGGGGCCACTAGGGACAGGATTGGCGAGGT
AGGCGTGTACGGTGGGAGGCCTATATAAGCAGAGCTCGTTTAGTGAACCG
TCAGATCGCCTGGAGAATTCgccaccatgGACTACAAGGATGACGACGAT
AAAACTTCCGGTGGCGGACTGGGTTCCACCGTGAGCAAGGGCGAGGAGGT
CATCAAAGAGTTCATGCGCTTCAAGGTGCGCATGGAGGGCTCCATGAACG
GCCACGAGTTCGAGATCGAGGGCGAGGGCGAGGGCCGCCCCTACGAGGGC
ACCCAGACCGCCAAGCTGAAGGTGACCAAGGGCGGCCCCCTGCCCTTCGC
CTGGGACATCCTGTCCCCCCAGTTCATGTACGGCTCCAAGGCGTACGTGA
AGCACCCCGCCGACATCCCCGATTACAAGAAGCTGTCCTTCCCCGAGGGC
TTCAAGTGGGAGCGCGTGATGAACTTCGAGGACGGCGGTCTGGTGACCGT
GACCCAGGACTCCTCCCTGCAGGACGGCACGCTGATCTACAAGGTGAAGA
TGCGCGGCACCAACTCCCCCCCGACGGCCCCGTAATGCAGAAGAAGACCA
TGGGCTGGGAGGCCTCCACCGAGCGCCTGTACCCCCGCGACGGCGTGCTG
AAGGGCGAGATCCACCAGGCCCTGAAGCGAAGGACGGCGGCCACTACCTG
GTGGAGTTCAAGACCATCTACATGGCCAAGAAGCCCGTGCAACGCCCGGC
TACTACTACGTGGACACCAAGCTGGACATCACCTCCCACAACGAGGACTA
CACCATCGTGGAACAGTACGAGCGCTCCGAGGGCCGCCACCACCTGTTCC
TGTACGGCATGGACGAGCTGTACAAGTAA

D. General format of the reporter libraries used for TALE and Cas9-gRNA specificity assays is provided below (ISceI control-TF target, gRNA/TALE target site (23 bp for gRNAs and 18 bp for TALEs), minCMV promoter. RNA barcode, and dTomato sequences are highlighted).

### Specificity Reporter Libraries

(SEQ ID NO: 10)
TAGGGATAACAGGGTAATAGTNNNNNNNNNNNNNNNNNNNNNNNCGAGGT
AGGCGTGTACGGTGGGAGGCCTATATAAGCAGAGCTCGTTTAGTGAACCG
TCAGATCGCCTGGAGAATTCgccaccatgGACTACAAGGATGACGACGAT
AAANNNNNNNNNNNNNNNNNNNNNNNNACTTCCGGTGGCGGACTGGGTTC
CACCGTGAGCAAGGGCGAGGAGGTCATCAAAGAGTTCATGCGCTTCAAGG
TGCGCATGGAGGGCTCCATGAACGGCCACGAGTTCGAGATCGAGGGCGAG
GGCGAGGGCCGCCCCTACGAGGGCACCCAGACCGCCAAGCTGAAGGTGAC
CAAGGGCGGCCCCCTGCCCTTCGCCTGGGACATCCTGTCCCCCCAGTTCA
TGTACGGCTCCAAGGCGTACGTGAAGCACCCCGCCGACATCCCCGATACA
AGAAGCTGTCCTCCCCGAGGGCTTCAAGTGGGAGCGCGTGATGAACTTCG
AGGACGGCGGTCTGGTGACCGTGACCCAGGACTCCTCCCTGCAGGACGGC
ACGCTGATCTACAAGGTGAAGATGCGCGGCACCAACTTCCCCCCCGACGG
CCCCGTAATGCAGAAGAAGACCATGGGCTGGGAGGCCTCCACCGAGCGCC
TGTACCCCCGCGACGGCGTGCTGAAGGGCGAGATCCACCAGGCCCTGAAG
CTGAAGGACGGCGGCCACTACCTGGTGGAGTTCAAGACCATCTACATGGC
CAAGAAGCCCGTGCAACTGCCCGGCTACTACTACGTGGACACCAAGCTGG
ACATCACCTCCCACAACGAGGACTACACCATCGTGGAACAGTACGAGCGC
TCCGAGGGCCGCCACCACCTGTTCCTGTACGGCATGGACGAGCTGTACAA
GTAAGAATTC

1. A method of localizing an effector domain to a target nucleic acid sequence in a eukaryotic cell comprising
providing to the cell a guide RNA complementary to the target nucleic acid sequence and an aptamer comprising a target of an RNA binding domain, wherein the guide RNA is a tracrRNA-crRNA fusion,
providing to the cell a nucleic acid encoding the effector domain and an RNA binding domain, wherein the RNA binding domain binds to the target of the RNA binding domain,
providing to the cell a nucleic acid encoding a nuclease null or nickase Cas9 protein that interacts with the guide RNA, and
wherein the cell expresses the effector domain having the RNA binding domain and the Cas9 protein, and
wherein the guide RNA including the effector domain connected thereto and the Cas9 protein co-localize to the target nucleic acid sequence.

2. The method of claim 1 wherein the cell is a yeast cell, a plant cell or a mammalian cell.

3. The method or claim 1 wherein the cell is a human cell.

4. The method of claim 1 wherein the guide RNA is between about 10 to about 250 nucleotides.

5. The method of claim 1 wherein the guide RNA is between about 20 to about 100 nucleotides.

6. The method of claim 1 wherein the guide RNA is between about 100 to about 250 nucleotides.

7. The method of claim 1 wherein the target nucleic acid sequence is genomic DNA, mitochondrial DNA, viral DNA or exogenous DNA.

8. The method of claim 1 wherein the aptamer comprises two copies of MS2 bacteriophage coat-protein binding RNA stem-loop.

9. The method of claim 1 wherein the RNA binding domain comprises MS2 bacteriophage coat-protein.

10. The method of claim 1 wherein multiple guide RNAs are provided to the cell with each guide RNA being complementary to a different target nucleic acid sequence and having an aptamer comprising a target of an RNA binding domain, wherein each guide RNA is a tracrRNA-crRNA fusion, and wherein multiple guide RNAs have the effector domain connected thereto.

11. A method of editing a target gene in a eukaryotic cell comprising
providing to the cell a guide RNA complementary to a target DNA sequence within or adjacent to the target gene and an aptamer comprising a target of an RNA binding domain, wherein the guide RNA is a tracrRNA-crRNA fusion,
providing to the cell a nucleic acid encoding an effector domain and an RNA binding domain, wherein the RNA binding domain binds to the target of the RNA binding domain,
providing to the cell a nucleic acid encoding a nuclease null or nickase Cas9 protein that interacts with the guide RNA, and
wherein the cell expresses the effector domain having the RNA binding domain and the Cas9 protein, and
wherein the guide RNA including the effector domain connected thereto and the Cas9 protein co-localize to the target DNA sequence and thereby edit the target gene.

12. The method of claim 11 wherein the cell is a yeast cell, a plant cell or a mammalian cell.

13. The method or claim 11 wherein the cell is a human cell.

14. The method of claim 11 wherein the guide RNA is between about 10 to about 250 nucleotides.

15. The method of claim 11 wherein the guide RNA is between about 20 to about 100 nucleotides.

16. The method of claim 11 wherein the guide RNA is between about 100 to about 250 nucleotides.

17. The method of claim 11 wherein the target DNA sequence is genomic DNA, mitochondrial DNA, viral DNA or exogenous DNA.

18. The method of claim 11 wherein the aptamer comprises two copies of MS2 bacteriophage coat-protein binding RNA stem-loop.

19. The method of claim 11 wherein the RNA binding domain comprises MS2 bacteriophage coat-protein.

20. The method of claim 11 wherein multiple guide RNAs are provided to the cell with each guide RNA being complementary to a different target nucleic acid sequence and having an aptamer comprising a target of an RNA binding domain, wherein each guide RNA is a tracrRNA-crRNA fusion, and wherein multiple guide RNAs have the effector domain connected thereto.

21. The method of claim 1 wherein the aptamer is attached to the 3′ end of the guide RNA.

22. The method of claim 1 wherein wherein the aptamer is attached to the 5′ end of the guide RNA.

23. The method of claim 10 wherein the aptamer is attached to the 3′ end of the guide RNA.

24. The method of claim 10 wherein wherein the aptamer is attached to the 5′ end of the guide RNA.

25. The method of claim 11 wherein the aptamer is attached to the 3′ end of the guide RNA.

26. The method of claim 11 wherein wherein the aptamer is attached to the 5′ end of the guide RNA.

27. The method of claim 20 wherein the aptamer is attached to the 3′ end of the guide RNA.

28. The method of claim 20 wherein wherein the aptamer is attached to the 5′ end of the guide RNA.


SEQUENCE LISTING (187 sequences)

SEQ ID NO: 1 - protein, 1368 aa, Streptococcus pyogenes [sequence omitted]

SEQ ID NO: 2 - DNA, 4332 bp, synthetic construct
  misc_feature 1..4332: note = VP64-activator construct
  source 1..4332: mol_type = other DNA
gccaccatggacaagaagtactccattgggctcgctatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggctgctatcgtgccccagtcttttctcaaagatgattctattgataataaagtgttgacaagatccgataaagctagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtggaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagatga

SEQ ID NO: 3 - DNA, 4365 bp, synthetic construct
  misc_feature 1..4365: note = VP64-activator construct
  source 1..4365: mol_type = other DNA
gccaccatgcccaagaagaagaggaaggtgggaagggggatggacaagaagtactccattgggctcgctatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggctgctatcgtgccccagtcttttctcaaagatgattctattgataataaagtgttgacaagatccgataaagctagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtggaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagatga

SEQ ID NO: 4 - DNA, 4425 bp, synthetic construct
  misc_feature 1..4425: note = VP64-activator construct
  source 1..4425: mol_type = other DNA
gccaccatggacaagaagtactccattgggctcgctatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggctgctatcgtgccccagtcttttctcaaagatgattctattgataataaagtgttgacaagatccgataaagctagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtggaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagagcggccgcagatccaaaaaagaagagaaaggtagatccaaaaaagaagagaaaggtagatccaaaaaagaagagaaaggtagatacggccgcatag

SEQ ID NO: 5 - DNA, 587 bp, synthetic construct
  misc_feature 1..587: note = MS2-activator construct
  source 1..587: mol_type = other DNA
ccaccatgggacctaagaaaaagaggaaggtggcggccgcttctagaatggcttctaactttactcagttcgttctcgtcgacaatggcggaactggcgacgtgactgtcgccccaagcaacttcgctaacgggatcgctgaatggatcagctctaactcgcgttcacaggcttacaaagtaacctgtagcgttcgtcagagctctgcgcagaatcgcaaatacaccatcaaagtcgaggtgcctaaaggcgcctggcgttcgtacttaaatatggaactaaccattccaattttcgccacgaattccgactgcgagcttattgttaaggcaatgcaaggtctcctaaaagatggaaacccgattccctcagcaatcgcagcaaactccggcatctacgaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagatga

SEQ ID NO: 6 - DNA, 681 bp, synthetic construct
  misc_feature 1..681: note = MS2-activator construct
  source 1..681: mol_type = other DNA
gccaccatgggacctaagaaaaagaggaaggtggcggccgcttctagaatggcttctaactttactcagttcgttctcgtcgacaatggcggaactggcgacgtgactgtcgccccaagcaacttcgctaacgggatcgctgaatggatcagctctaactcgcgttcacaggcttacaaagtaacctgtagcgttcgtcagagctctgcgcagaatcgcaaatacaccatcaaagtcgaggtgcctaaaggcgcctggcgttcgtacttaaatatggaactaaccattccaattttcgccacgaattccgactgcgagcttattgttaaggcaatgcaaggtctcctaaaagatggaaacccgattccctcagcaatcgcagcaaactccggcatctacgaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagagcggccgcagatccaaaaaagaagagaaaggtagatccaaaaaagaagagaaaggtagatccaaaaaagaagagaaaggtagatacggccgcatag

SEQ ID NO: 7 - DNA, 557 bp, synthetic construct
  variation 320..339: note = wherein N is G, A, T or C
  source 1..557: mol_type = other DNA
tgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgnnnnnnnnnnnnnnnnnnnngttttagagctagaaatagcaagttaaaataaggctagtccgttatcaacttgaaaaagtggcaccgagtcggtgctctgcaggtcgactctagaaaacatgaggatcacccatgtctgcagtattcccgggttcattagatcctaaggtacctaattgcctagaaaacatgaggatcacccatgtctgcaggtcgactctagaaattttttctagac

SEQ ID NO: 8 - DNA, 882 bp, synthetic construct
  misc_feature 1..882: note = Activation reporter construct
  source 1..882: mol_type = other DNA
tagggataacagggtaatagtgtcccctccaccccacagtggggcgaggtaggcgtgtacggtgggaggcctatataagcagagctcgtttagtgaaccgtcagatcgcctggagaattcgccaccatggactacaaggatgacgacgataaaacttccggtggcggactgggttccaccgtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagtaa

SEQ ID NO: 9 - DNA, 882 bp, synthetic construct
  misc_feature 1..882: note = Activation reporter construct
  source 1..882: mol_type = other DNA
tagggataacagggtaatagtggggccactagggacaggattggcgaggtaggcgtgtacggtgggaggcctatataagcagagctcgtttagtgaaccgtcagatcgcctggagaattcgccaccatggactacaaggatgacgacgataaaacttccggtggcggactgggttccaccgtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagtaa

SEQ ID NO: 10 - DNA, 912 bp, synthetic construct
  misc_feature 1..912: note = Specificity reporter library
  variation 22..44: note = wherein N is G, A, T or C
  variation 154..177: note = wherein N is G, A, T or C
  source 1..912: mol_type = other DNA
tagggataacagggtaatagtnnnnnnnnnnnnnnnnnnnnnnncgaggtaggcgtgtacggtgggaggcctatataagcagagctcgtttagtgaaccgtcagatcgcctggagaattcgccaccatggactacaaggatgacgacgataaannnnnnnnnnnnnnnnnnnnnnnnacttccggtggcggactgggttccaccgtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagtaagaattc

SEQ ID NO: 11 - DNA, 23 bp, synthetic construct
  misc_feature 1..23: note = Target probe
  source 1..23: mol_type = other DNA
ctggcggatcactcgcggttagg

SEQ ID NO: 12 - DNA, 23 bp, synthetic construct
  misc_feature 1..23: note = Target probe
  source 1..23: mol_type = other DNA
cctcggcctccaaaagtgctagg

SEQ ID NO: 13 - DNA, 23 bp, synthetic construct
  misc_feature 1..23: note = Target probe
  source 1..23: mol_type = other DNA
acgctgattcctgcagatcaggg

SEQ ID NO: 14 - DNA, 23 bp, synthetic construct
  misc_feature 1..23: note = Target probe
  source 1..23: mol_type = other DNA
ccaggaatacgtatccaccaggg

SEQ ID NO: 15 - DNA, 23 bp, synthetic construct
  misc_feature 1..23: note = Target probe
  source 1..23: mol_type = other DNA
gccacacccaagcgatcaaatgg

SEQ ID NO: 16 - DNA, 23 bp, synthetic construct
  misc_feature 1..23: note = Target probe
  source 1..23: mol_type = other DNA
aaataatacattctaaggtaagg

SEQ ID NO: 17 - DNA, 23 bp, synthetic construct
  misc_feature 1..23: note = Target probe
  source 1..23: mol_type = other DNA
gctactggggaggctgaggcagg

SEQ ID NO: 18 - DNA, 23 bp, synthetic construct
  misc_feature 1..23: note = Target probe
  source 1..23: mol_type = other DNA
tagcaatacagtcacattaatgg

SEQ ID NO: 19 - DNA, 23 bp, synthetic construct
  misc_feature 1..23: note = Target probe
  source 1..23: mol_type = other DNA
ctcatgtgatccccccgtctcgg

SEQ ID NO: 20 - DNA, 23 bp, synthetic construct
  misc_feature 1..23: note = Target probe
  source 1..23: mol_type = other DNA
ccgggcagagagtgaacgcgcgg

SEQ ID NO: 21 - DNA, 23 bp, synthetic construct
  misc_feature 1..23: note = Target probe
  source 1..23: mol_type = other DNA
ttccttccctctcccgtgcttgg

SEQ ID NO: 22 - DNA, 23 bp, synthetic construct
  misc_feature 1..23: note = Target probe
  source 1..23: mol_type = other DNA
tctctgcaaagcccctggagagg

SEQ ID NO: 23 - DNA, 23 bp, synthetic construct
  misc_feature 1..23: note = Target probe
  source 1..23: mol_type = other DNA
aatgcagttgccgagtgcagtgg

SEQ ID NO: 24 - DNA, 23 bp, synthetic construct
  misc_feature 1..23: note = Target probe
  source 1..23: mol_type = other DNA
cctcagcctcctaaagtgctggg

SEQ ID NO: 25 - DNA, 23 bp, synthetic construct
  misc_feature 1..23: note = Target probe
  source 1..23: mol_type = other DNA
gagtccaaatcctctttactagg

SEQ ID NO: 26 - DNA, 23 bp, synthetic construct
  misc_feature 1..23: note = Target probe
  source 1..23: mol_type = other DNA
gagtgtctggatttgggataagg

SEQ ID NO: 27 - DNA, 23 bp, synthetic construct
  misc_feature 1..23: note = Target probe
  source 1..23: mol_type = other DNA
cagcacctcatctcccagtgagg

SEQ ID NO: 28 - DNA, 23 bp, synthetic construct
  misc_feature 1..23: note = Target probe
  source 1..23: mol_type = other DNA
tctaaaacccagggaatcatggg

SEQ ID NO: 29 - DNA, 23 bp, synthetic construct
  misc_feature 1..23: note = Target probe
  source 1..23: mol_type = other DNA
cacaaggcagccagggatccagg

SEQ ID NO: 30 - DNA, 23 bp, synthetic construct
  misc_feature 1..23: note = Target probe
  source 1..23: mol_type = other DNA
gatggcaagctgagaaacactgg

SEQ ID NO: 31 - DNA, 23 bp, synthetic construct
  misc_feature 1..23: note = Target probe
  source 1..23: mol_type = other DNA
tgaaatgcacgcatacaattagg

SEQ ID NO: 32 - DNA, 23 bp, synthetic construct
  misc_feature 1..23: note = Target probe
  source 1..23: mol_type = other DNA
ccagtccagacctggccttctgg

SEQ ID NO: 33 - DNA, 23 bp, synthetic construct
  misc_feature 1..23: note = Target probe
  source 1..23: mol_type = other DNA
cccagaaaaacagaccctgaagg

SEQ ID NO: 34 - DNA, 23 bp, synthetic construct
  misc_feature 1..23: note = Target probe
  source 1..23: mol_type = other DNA
aagggttgagcacttgtttaggg

SEQ ID NO: 35 - DNA, 23 bp, synthetic construct
  misc_feature 1..23: note = Target probe
  source 1..23: mol_type = other DNA
atgtctgagttttggttgagagg

SEQ ID NO: 36 - DNA, 23 bp, synthetic construct
  misc_feature 1..23: note = Target probe
  source 1..23: mol_type = other DNA
ggtcccttgaaggggaagtaggg

SEQ ID NO: 37 - DNA, 23 bp, synthetic construct
  misc_feature 1..23: note = Target probe
  source 1..23: mol_type = other DNA
tggcagtctactcttgaagatgg

SEQ ID NO: 38 - DNA, 23 bp, synthetic construct
  misc_feature 1..23: note = Target probe
  source 1..23: mol_type = other DNA
ggcacagtgccagaggtctgtgg

SEQ ID NO: 39 - DNA, 23 bp, synthetic construct
  misc_feature 1..23: note = Target probe
  source 1..23: mol_type = other DNA
taaaaataaaaaaactaacaggg

SEQ ID NO: 40 - DNA, 23 bp, synthetic construct
  misc_feature 1..23: note = Target probe
  source 1..23: mol_type = other DNA
tctgtgggggacctgcactgagg

SEQ ID NO: 41 - DNA, 23 bp, synthetic construct
  misc_feature 1..23: note = Target probe
  source 1..23: mol_type = other DNA
ggccagaggtcaaggctagtggg

SEQ ID NO: 42 - DNA, 23 bp, synthetic construct
  misc_feature 1..23: note = Target probe
  source 1..23: mol_type = other DNA
cacgaccgaaacccttcttacgg

SEQ ID NO: 43 - DNA, 23 bp, synthetic construct
  misc_feature 1..23: note = Target probe
  source 1..23: mol_type = other DNA
gttgaatgaagacagtctagtgg

SEQ ID NO: 44 - DNA, 23 bp, synthetic construct
  misc_feature 1..23: note = Target probe
  source 1..23: mol_type = other DNA
taagaacagagcaagttacgtgg

SEQ ID NO: 45 - DNA, 23 bp, synthetic construct
  misc_feature 1..23: note = Target probe
  source 1..23: mol_type = other DNA
tgtaaggtaagagaggagagcgg

SEQ ID NO: 46 - DNA, 23 bp, synthetic construct
  misc_feature 1..23: note = Target probe
  source 1..23: mol_type = other DNA
tgacacaccaactcctgcactgg

SEQ ID NO: 47 - DNA, 23 bp, synthetic construct
  misc_feature 1..23: note = Target probe
  source 1..23: mol_type = other DNA
tttacccacttccttcgaaaagg

SEQ ID NO: 48 - DNA, 23 bp, synthetic construct
  misc_feature 1..23: note = Target probe
  source 1..23: mol_type = other DNA
gtggctggcaggctggctctggg

SEQ ID NO: 49 - DNA, 23 bp, synthetic construct
  misc_feature 1..23: note = Target probe
  source 1..23: mol_type = other DNA
ctcccccggcctcccccgcgcgg

SEQ ID NO: 50 - DNA, 23 bp, synthetic construct
  misc_feature 1..23: note = Target probe
  source 1..23: mol_type = other DNA
caaaacccggcagcgaggctggg

SEQ ID NO: 51 - DNA, 23 bp, synthetic construct
  misc_feature 1..23: note = Target probe
  source 1..23: mol_type = other DNA
aggagccgccgcgcgctgattgg

SEQ ID NO: 52 - DNA, 23 bp, synthetic construct
  misc_feature 1..23: note = Target probe
  source 1..23: mol_type = other DNA
cacacacacccacacgagatggg

SEQ ID NO: 53 - DNA, 23 bp, synthetic construct
  misc_feature 1..23: note = Target probe
  source 1..23: mol_type = other DNA
gaagaagctaaagagccagaggg

SEQ ID NO: 54 - DNA, 23 bp, synthetic construct
  misc_feature 1..23: note = Target probe
  source 1..23: mol_type = other DNA
atgagaatttcaataacctcagg

SEQ ID NO: 55 - DNA, 23 bp, synthetic construct
  misc_feature 1..23: note = Target probe
  source 1..23: mol_type = other DNA
tcccgctctgttgcccaggctgg

SEQ ID NO: 56 - DNA, 23 bp, synthetic construct
  misc_feature 1..23: note = Target probe
  source 1..23: mol_type = other DNA
cagacacccaccaccatgcgtgg

SEQ ID NO: 57 - DNA, 23 bp, synthetic construct
  misc_feature 1..23: note = Target probe
  source 1..23: mol_type = other DNA
tcccaatttactgggattacagg

SEQ ID NO: 58 - DNA, 23 bp, synthetic construct
  misc_feature 1..23: note = Target probe
  source 1..23: mol_type = other DNA
tgatttaaaagttggaaacgtgg

SEQ ID NO: 59 - DNA, 23 bp, synthetic construct
  misc_feature 1..23: note = Target probe
  source 1..23: mol_type = other DNA
tctagttccccacctagtctggg

SEQ ID NO: 60 - DNA, 23 bp, synthetic construct
  misc_feature 1..23: note = Target probe
  source 1..23: mol_type = other DNA
gattaactgagaattcacaaggg

SEQ ID NO: 61 - DNA, 23 bp, synthetic construct
  misc_feature 1..23: note = Target probe
  source 1..23: mol_type = other DNA
cgccaggaggggtgggtctaagg

SEQ ID NO: 62 - DNA, 23 bp, synthetic construct
  misc_feature 1..23: note = Reporter construct
  source 1..23: mol_type = other DNA
gtcccctccaccccacagtgggg

SEQ ID NO: 63 - DNA, 23 bp, synthetic construct
  misc_feature 1..23: note = Reporter construct
  source 1..23: mol_type = other DNA
ggggccactagggacaggattgg

SEQ ID NO: 64 - DNA, 71 bp, synthetic construct
  misc_feature 1..71: note = Target oligonucleotide sequence
  source 1..71: mol_type = other DNA
taatacttttatctgtcccctccaccccacagtggggccactagggacaggattggtgacagaaaagcccc

SEQ ID NO: 65 - DNA, 20 bp, synthetic construct
  misc_feature 1..20: note = Target oligonucleotide sequence
  source 1..20: mol_type = other DNA
ggggccactagggacaggat

SEQ ID NO: 66 - RNA, 80 nt, synthetic construct
  misc_feature 1..80: note = Guide RNA
  source 1..80: mol_type = other RNA
gttttagagctagaaatagcaagttaaaataaggctagtccgttatcaacttgaaaaagtggcaccgagtcggtgctttt

SEQ ID NO: 67 - DNA, 23 bp, synthetic construct
  misc_feature 1..23: note = Target oligonucleotide sequence
  source 1..23: mol_type = other DNA
gtcccctccaccccacagtgcag

SEQ ID NO: 68 - DNA, 23 bp, synthetic construct
  misc_feature 1..23: note = Target oligonucleotide sequence
  source 1..23: mol_type = other DNA
gtcccctccaccccacagtgcaa

SEQ ID NO: 69 - DNA, 23 bp, synthetic construct
  misc_feature 1..23: note = Target oligonucleotide sequence
  source 1..23: mol_type = other DNA
gtcccctccaccccacagtgcgg

SEQ ID NO: 70 - DNA, 52 bp, synthetic construct
  misc_feature 1..52: note = Target oligonucleotide sequence
  source 1..52: mol_type = other DNA
tgtcccctccaccccacagtggggccactagggacaggattggtgacagaaa

SEQ ID NO: 71 - DNA, 52 bp, synthetic construct
  misc_feature 1..52: note = Target oligonucleotide sequence
  source 1..52: mol_type = other DNA
tgtcccccccaccccacagtggggccactagggacaggattggtgacagaaa

SEQ ID NO: 72 - DNA, 52 bp, synthetic construct
  misc_feature 1..52: note = Target oligonucleotide sequence
  source 1..52: mol_type = other DNA
aaaaccctccaccccacagtggggccactagggacaggattggtgacagaaa

SEQ ID NO: 73 - DNA, 52 bp, synthetic construct
  misc_feature 1..52: note = Target oligonucleotide sequence
  source 1..52: mol_type = other DNA
tgtcccctccttttttcagtggggccactagggacaggattggtgacagaaa

SEQ ID NO: 74 - DNA, 23 bp, synthetic construct
  misc_feature 1..23: note = Target oligonucleotide sequence
  source 1..23: mol_type = other DNA
caccggggtggtgcccatcctgg

SEQ ID NO: 75 - DNA, 23 bp, synthetic construct
  misc_feature 1..23: note = Target oligonucleotide sequence
  source 1..23: mol_type = other DNA
ggtgcccatcctggtcgagctgg

SEQ ID NO: 76 - DNA, 23 bp, synthetic construct
  misc_feature 1..23: note = Target oligonucleotide sequence
  source 1..23: mol_type = other DNA
cccatcctggtcgagctggacgg

SEQ ID NO: 77 - DNA, 23 bp, synthetic construct
  misc_feature 1..23: note = Target oligonucleotide sequence
  source 1..23: mol_type = other DNA
ggccacaagttcagcgtgtccgg

SEQ ID NO: 78 - DNA, 23 bp, synthetic construct
  misc_feature 1..23: note = Target oligonucleotide sequence
  source 1..23: mol_type = other DNA
cgcaaataagagctcacctacgg

SEQ ID NO: 79 - DNA, 23 bp, synthetic construct
  misc_feature 1..23: note = Target oligonucleotide sequence
  source 1..23: mol_type = other DNA
ctgaagttcatctgcaccaccgg

SEQ ID NO: 80 - DNA, 23 bp, synthetic construct
  misc_feature 1..23: note = Target oligonucleotide sequence
  source 1..23: mol_type = other DNA
ccggcaagctgcccgtgccctgg

SEQ ID NO: 81 - DNA, 23 bp, synthetic construct
  misc_feature 1..23: note = Target oligonucleotide sequence
  source 1..23: mol_type = other DNA
gaccaggatgggcaccaccccgg

SEQ ID NO: 82 - DNA, 23 bp, synthetic construct
  misc_feature 1..23: note = Target oligonucleotide sequence
  source 1..23: mol_type = other DNA
gccgtccagctcgaccaggatgg

SEQ ID NO: 83 - DNA, 23 bp, synthetic construct
  misc_feature 1..23: note = Target oligonucleotide sequence
  source 1..23: mol_type = other DNA
ggccggacacgctgaacttgtgg

SEQ ID NO: 84 - DNA, 23 bp, synthetic construct
  misc_feature 1..23: note = Target oligonucleotide sequence
  source 1..23: mol_type = other DNA
taacagggtaatgtcgaggccgg

SEQ ID NO: 85 - DNA, 23 bp, synthetic construct
  misc_feature 1..23: note = Target oligonucleotide sequence
  source 1..23: mol_type = other DNA
aggtgagctcttatttgcgtagg

SEQ ID NO: 86 - DNA, 23 bp, synthetic construct
  misc_feature 1..23: note = Target oligonucleotide sequence
  source 1..23: mol_type = other DNA
cttcagggtcagcttgccgtagg

SEQ ID NO: 87 - DNA, 23 bp, synthetic construct
  misc_feature 1..23: note = Target oligonucleotide sequence
  source 1..23: mol_type = other DNA
gggcacgggcagcttgccggtgg

SEQ ID NO: 88 - DNA, 23 bp, synthetic construct
  misc_feature 1..23: note = Target oligonucleotide sequence
  source 1..23: mol_type = other DNA
gagatgatcgccccttcttctgg

SEQ ID NO: 89 - DNA, 20 bp, synthetic construct
  misc_feature 1..20: note = Target oligonucleotide sequence
  source 1..20: mol_type = other DNA
gagatgatcgccccttcttc

SEQ ID NO: 90 - DNA, 20 bp, synthetic construct
  misc_feature 1..20: note = Target oligonucleotide sequence
  source 1..20: mol_type = other DNA
gtgatgaccggccgttcttc

SEQ ID NO: 91 - DNA, 23 bp, synthetic construct
  misc_feature 1..23: note = Target oligonucleotide sequence
  source 1..23: mol_type = other DNA
gtcccctccaccccacagtgggg

SEQ ID NO: 92 - DNA, 23 bp, synthetic construct
  misc_feature 1..23: note = Target oligonucleotide sequence
  source 1..23: mol_type = other DNA
gagatgatcgcccgttcttctgg

SEQ ID NO: 93 - RNA, 20 nt, synthetic construct
  misc_feature 1..20: note = RNA target sequence
  source 1..20: mol_type = other RNA
gtcccctccaccccacagtg

SEQ ID NO: 94 - RNA, 20 nt, synthetic construct
  misc_feature 1..20: note = RNA target sequence
  source 1..20: mol_type = other RNA
gtcccctccaccccacagtc

SEQ ID NO: 95 - RNA, 20 nt, synthetic construct
  misc_feature 1..20: note = RNA target sequence
  source 1..20: mol_type = other RNA
gtcccctccaccccacagag

SEQ ID NO: 96 - RNA, 20 nt, synthetic construct
  misc_feature 1..20: note = RNA target sequence
  source 1..20: mol_type = other RNA
gtcccctccaccccacactg

SEQ ID NO: 97 - RNA, 20 nt, synthetic construct
  misc_feature 1..20: note = RNA target sequence
  source 1..20: mol_type = other RNA
gtcccctccaccccactgtg

SEQ ID NO: 98 - RNA, 20 nt, synthetic construct
  misc_feature 1..20: note = RNA target sequence
  source 1..20: mol_type = other RNA
gtcccctccaccccagagtg

SEQ ID NO: 99 - RNA, 20 nt, synthetic construct
  misc_feature 1..20: note = RNA target sequence
  source 1..20: mol_type = other RNA
gtcccctccacccctcagtg

SEQ ID NO: 100 - RNA, 20 nt, synthetic construct
  misc_feature 1..20: note = RNA target sequence
  source 1..20: mol_type = other RNA
gtcccctccacccgacagtg

SEQ ID NO: 101 - RNA, 20 nt, synthetic construct
  misc_feature 1..20: note = RNA target sequence
  source 1..20: mol_type = other RNA
gtcccctccaccgcacagtg

SEQ ID NO: 102 - RNA, 20 nt, synthetic construct
  misc_feature 1..20: note = RNA target sequence
  source 1..20: mol_type = other RNA
gtcccctccacgccacagtg

SEQ ID NO: 103 - RNA, 20 nt, synthetic construct
  misc_feature 1..20: note = RNA target sequence
  source 1..20: mol_type = other RNA
gtcccctccagcccacagtg

SEQ ID NO: 104 - RNA, 20 nt, synthetic construct
  misc_feature 1..20: note = RNA target sequence
  source 1..20: mol_type = other RNA
gtcccctcctccccacagtg

SEQ ID NO: 105 - RNA, 20 nt, synthetic construct
  misc_feature 1..20: note = RNA target sequence
  source 1..20: mol_type = other RNA
gtcccctcgaccccacagtg

SEQ ID NO: 106 - RNA, 20 nt, synthetic construct
  misc_feature 1..20: note = RNA target sequence
  source 1..20: mol_type = other RNA
gtcccctccaccccacagac

SEQ ID NO: 107 - RNA, 20 nt, synthetic construct
  misc_feature 1..20: note = RNA target sequence
  source 1..20: mol_type = other RNA
gtcccctccaccccactctg

SEQ ID NO: 108 - RNA, 20 nt, synthetic construct
  misc_feature 1..20: note = RNA target sequence
  source 1..20: mol_type = other RNA
gtcccctccacccctgagtg

SEQ ID NO: 109 - RNA, 20 nt, synthetic construct
  misc_feature 1..20: note = RNA target sequence
  source 1..20: mol_type = other RNA
gtcccctccaccggacagtg

SEQ ID NO: 110 - RNA, 20 nt, synthetic construct
  misc_feature 1..20: note = RNA target sequence
  source 1..20: mol_type = other RNA
gtcccctccaggccacagtg

SEQ ID NO: 111 - RNA, 20 nt, synthetic construct
  misc_feature 1..20: note = RNA target sequence
  source 1..20: mol_type = other RNA
gtcccctcgtccccacagtg

SEQ ID NO: 112 - DNA, 23 bp, synthetic construct
  misc_feature 1..23: note = Target oligonucleotide sequence
  source 1..23: mol_type = other DNA
ggggccactagggacaggatggg

SEQ ID NO: 113 - RNA, 20 nt, synthetic construct
  misc_feature 1..20: note = RNA target sequence
  source 1..20: mol_type = other RNA
gagatgatcgccccttcttc

SEQ ID NO: 114 - RNA, 20 nt, synthetic construct
  misc_feature 1..20: note = RNA target sequence
  source 1..20: mol_type = other RNA
gagatgatcgccccttcttg

SEQ ID NO: 115 - RNA, 20 nt, synthetic construct
  misc_feature 1..20: note = RNA target sequence
  source 1..20: mol_type = other RNA
gagatgatcgccccttctac

SEQ ID NO: 116 - RNA, 20 nt, synthetic construct
  misc_feature 1..20: note = RNA target sequence
  source 1..20: mol_type = other RNA
gagatgatcgccccttcatc

SEQ ID NO: 117 - RNA, 20 nt, synthetic construct
  misc_feature 1..20: note = RNA target sequence
  source 1..20: mol_type = other RNA
gagatgatcgccccttgttc

SEQ ID NO: 118 - RNA, 20 nt, synthetic construct
  misc_feature 1..20: note = RNA target sequence
  source 1..20: mol_type = other RNA
gagatgatcgcccctacttc

SEQ ID NO: 119 - RNA, 20 nt, synthetic construct
  misc_feature 1..20: note = RNA target sequence
  source 1..20: mol_type = other RNA
gagatgatcgccccatcttc

SEQ ID NO: 120 - RNA, 20 nt, synthetic construct
  misc_feature 1..20: note = RNA target sequence
  source 1..20: mol_type = other RNA
gagatgatcgcccgttcttc

SEQ ID NO: 121 - RNA, 20 nt, synthetic construct
  misc_feature 1..20: note = RNA target sequence
  source 1..20: mol_type = other RNA
gagatgatcgccgcttcttc

SEQ ID NO: 122 - RNA, 20 nt, synthetic construct
  misc_feature 1..20: note = RNA target sequence
  source 1..20: mol_type = other RNA
gagatgatcgcgccttcttc

SEQ ID NO: 123 - RNA, 20 nt, synthetic construct
  misc_feature 1..20: note = RNA target sequence
  source 1..20: mol_type = other RNA
gagatgatcggcccttcttc

SEQ ID NO: 124 - RNA, 20 nt, synthetic construct
  misc_feature 1..20: note = RNA target sequence
  source 1..20: mol_type = other RNA
gagatgatccccccttcttc

SEQ ID NO: 125 - RNA, 20 nt, synthetic construct
  misc_feature 1..20: note = RNA target sequence
  source 1..20: mol_type = other RNA
gagatgatggccccttcttc

SEQ ID NO: 126 - RNA, 20 nt, synthetic construct
  misc_feature 1..20: note = RNA target sequence
  source 1..20: mol_type = other RNA
gagatgatcgccccttctag

SEQ ID NO: 127 - RNA, 20 nt, synthetic construct
  misc_feature 1..20: note = RNA target sequence
  source 1..20: mol_type = other RNA
gagatgatcgccccttgatc

SEQ ID NO: 128 - RNA, 20 nt, synthetic construct
  misc_feature 1..20: note = RNA target sequence
  source 1..20: mol_type = other RNA
gagatgatcgccccaacttc

SEQ ID NO: 129 - RNA, 20 nt, synthetic construct
  misc_feature 1..20: note = RNA target sequence
  source 1..20: mol_type = other RNA
gagatgatcgccggttcttc

SEQ ID NO: 130 - RNA, 20 nt, synthetic construct
  misc_feature 1..20: note = RNA target sequence
  source 1..20: mol_type = other RNA
gagatgatcgggccttcttc

SEQ ID NO: 131 - RNA, 20 nt, synthetic construct
  misc_feature 1..20: note = RNA target sequence
  source 1..20: mol_type = other RNA
gagatgatgcccccttcttc

SEQ ID NO: 132 - DNA, 23 bp, synthetic construct
  misc_feature 1..23: note = Target oligonucleotide sequence
  source 1..23: mol_type = other DNA
gagatgatcgccccttcttctgg

SEQ ID NO: 133 - RNA, 20 nt, synthetic construct
  misc_feature 1..20: note = RNA target sequence
  source 1..20: mol_type = other RNA
ggggccactagggacaggat

SEQ ID NO: 134 - RNA, 19 nt, synthetic construct
  misc_feature 1..19: note = RNA target sequence
  source 1..19: mol_type = other RNA
gggccactagggacaggat

SEQ ID NO: 135 - RNA, 18 nt, synthetic construct
  misc_feature 1..18: note = RNA target sequence
  source 1..18: mol_type = other RNA
ggccactagggacaggat

SEQ ID NO: 136 - RNA, 17 nt, synthetic construct
  misc_feature 1..17: note = RNA target sequence
  source 1..17: mol_type = other RNA
gccactagggacaggat

SEQ ID NO: 137 - RNA, 20 nt, synthetic construct
  misc_feature 1..20: note = RNA target sequence
  source 1..20: mol_type = other RNA
gagatgatcgccccttcttc

SEQ ID NO: 138 - RNA, 18 nt, synthetic construct
  misc_feature 1..18: note = RNA target sequence
  source 1..18: mol_type = other RNA
gatgatcgccccttcttc

SEQ ID NO: 139 - RNA, 15 nt, synthetic construct
  misc_feature 1..15: note = RNA target sequence
  source 1..15: mol_type = other RNA
gatcgccccttcttc

SEQ ID NO: 140 - RNA, 11 nt, synthetic construct
  misc_feature 1..11: note = RNA target sequence
  source 1..11: mol_type = other RNA
gccccttcttc

SEQ ID NO: 141 - DNA, 23 bp, synthetic construct
  misc_feature 1..23: note = Target oligonucleotide sequence
  source 1..23: mol_type = other DNA
gtcccctccaccccacagtgcrr

SEQ ID NO: 142 - skipped

SEQ ID NO: 143 - DNA, 14 bp, synthetic construct
  misc_feature 1..14: note = Target oligonucleotide sequence
  source 1..14: mol_type = other DNA
tgtcaaaaaaaccc

SEQ ID NO: 144 - DNA, 14 bp, synthetic construct
  misc_feature 1..14: note = Target oligonucleotide sequence
  source 1..14: mol_type = other DNA
tgtcggggggaccc

SEQ ID NO: 145 - DNA, 14 bp, synthetic construct
  misc_feature 1..14: note = Target oligonucleotide sequence
  source 1..14: mol_type = other DNA
tgtcaaaaaaaccc

SEQ ID NO: 146 - DNA, 14 bp, synthetic construct
  misc_feature 1..14: note = Target oligonucleotide sequence
  source 1..14: mol_type = other DNA
tgtcggggggaccc

SEQ ID NO: 147 - DNA, 14 bp, synthetic construct
  misc_feature 1..14: note = Target oligonucleotide sequence
  source 1..14: mol_type = other DNA
tgtcccccccaccc

SEQ ID NO: 148 - DNA, 14 bp, synthetic construct
  misc_feature 1..14: note = Target oligonucleotide sequence
  source 1..14: mol_type = other DNA
tgtcttttttaccc

SEQ ID NO: 149 - DNA, 14 bp, synthetic construct
  misc_feature 1..14: note = Target oligonucleotide sequence
  source 1..14: mol_type = other DNA
tgtcccccccaccc

SEQ ID NO: 150 - DNA, 14 bp, synthetic construct
  misc_feature 1..14: note = Target oligonucleotide sequence
  source 1..14: mol_type = other DNA
tgtcttttttaccc

SEQ ID NO: 151 - DNA, 23 bp, synthetic construct
  misc_feature 1..23: note = Target oligonucleotide sequence
  source 1..23: mol_type = other DNA
ggatcctgtgtccccgagctggg

SEQ ID NO: 152 - DNA, 23 bp, synthetic construct
  misc_feature 1..23: note = Target oligonucleotide sequence
  source 1..23: mol_type = other DNA
gttaatgtggctctggttctggg

SEQ ID NO: 153 - DNA, 23 bp, synthetic construct
  misc_feature 1..23: note = Target oligonucleotide sequence
  source 1..23: mol_type = other DNA
ggggccactagggacaggattgg

SEQ ID NO: 154 - DNA, 23 bp, synthetic construct
  misc_feature 1..23: note = Target oligonucleotide sequence
  source 1..23: mol_type = other DNA
cttcctagtctcctgatattggg

SEQ ID NO: 155 - DNA, 23 bp, synthetic construct
  misc_feature 1..23: note = Target oligonucleotide sequence
  source 1..23: mol_type = other DNA
tggtcccagctcggggacacagg

SEQ ID NO: 156 - DNA, 23 bp, synthetic construct
  misc_feature 1..23: note = Target oligonucleotide sequence
  source 1..23: mol_type = other DNA
agaaccagagccacattaaccgg

SEQ ID NO: 157 - DNA, 23 bp, synthetic construct
  misc_feature 1..23: note = Target oligonucleotide sequence
  source 1..23: mol_type = other DNA
gtcaccaatcctgtccctagtgg

SEQ ID NO: 158 - DNA, 23 bp, synthetic construct
  misc_feature 1..23: note = Target oligonucleotide sequence
  source 1..23: mol_type = other DNA
agacccaatatcaggagactagg

SEQ ID NO: 159 - DNA, 75 bp, synthetic construct
  misc_feature 1..75: note = Target oligonucleotide sequence
  source 1..75: mol_type = other DNA
gggatcctgtgtccccgagctgggaccaccttatattcccagggccggttaatgtggctctggttctgggtactt

SEQ ID NO: 160 - DNA, 69 bp, synthetic construct
  misc_feature 1..69: note = Target oligonucleotide sequence
  source 1..69: mol_type = other DNA
gggatcctgtgtccccgagctgggaccaccttatattcccagggccggttaatgtggttctgggtactt

SEQ ID NO: 161 - DNA, 113 bp, synthetic construct
  misc_feature 1..113: note = Target oligonucleotide sequence
  source 1..113: mol_type = other DNA
gggatcctgtgtccccgagctgggaccaccttatattcccagggcagggccggttggaccaccttatattcccagggcagggccggttaatgtggctctggttctgggtactt

SEQ ID NO: 162 - DNA, 34 bp, synthetic construct
  misc_feature 1..34: note = Target oligonucleotide sequence
  source 1..34: mol_type = other DNA
gggatcctgtgtccccgtctggttctgggtactt

SEQ ID NO: 163 - DNA, 47 bp, synthetic construct
  misc_feature 1..47: note = Target oligoncleotide sequence
  source 1..47: mol_type = other DNA
gggatcctgtgtccccgagctgggaccaccttatattctgggtactt

SEQ ID NO: 164 - DNA, 17 bp, synthetic construct
  misc_feature 1..17: note = Target oligonucleotide sequence
  source 1..17: mol_type = other DNA
gggatcctgtggtactt

SEQ ID NO: 165 - DNA, 93 bp, synthetic construct
  misc_feature 1..93: note = Target oligonucleotide sequence
  source 1..93: mol_type = other DNA
agggccggttaatgtggctctggttctgggtacttttatctgtcccctccaccccacagtggggccactagggacaggattggtgacagaaaa

SEQ ID NO: 166 - DNA, 83 bp, synthetic construct
  misc_feature 1..83: note = Target oligonucleotide sequence
  source 1..83: mol_type = other DNA
agggccggttaatgaatgtggctctggttctgggtacttttatctgtcccctccaccccacagtggggccactagacagaaaa

SEQ ID NO: 167 - DNA, 76 bp, synthetic construct
  misc_feature 1..76: note = Target oligonucleotide sequence
  source 1..76: mol_type = other DNA
agggccggttaatgtggctctggttctgggtacttttatctgtcccccagtggggccactgattggtgacagaaaa

SEQ ID NO: 168 - DNA, 29 bp, synthetic construct
  misc_feature 1..29: note = Target oligonucleotide sequence
  source 1..29: mol_type = other DNA
agggccggttcaggattggtgacagaaaa

SEQ ID NO: 169 - DNA, 34 bp, synthetic construct
  misc_feature 1..34: note = Target oligonucleotide sequence
  source 1..34: mol_type = other DNA
agggccggttaatgtggcgattggtgacagaaaa

SEQ ID NO: 170 - DNA, 63 bp, synthetic construct
  misc_feature 1..63: note = Target oligonucleotide sequence
  source 1..63: mol_type = other DNA
agggccggttaatgtggctctggttctgggtacttttatctgtccccgattggtgacagaaaa

SEQ ID NO: 171 - DNA, 84 bp, synthetic construct
  misc_feature 1..84: note = Target oligonucleotide sequence
  source 1..84: mol_type = other DNA
agggccggttaatgtggctctggttctgggtacttttatctgtcccctccaccccacagtggggacaggattggtgacagaaaa

SEQ ID NO: 172 - DNA, 27 bp, synthetic construct
  misc_feature 1..27: note = Target oligonucleotide sequence
  source 1..27: mol_type = other DNA
agggccggttaatgtggtgacagaaaa

SEQ ID NO: 173 - DNA, 105 bp, synthetic construct
  misc_feature 1..105: note = Target oligonucleotide sequence
  source 1..105: mol_type = other DNA
agggccggttaatgtggctctggttctgggtacttttatctgtcccctccaccccaggggacagtctgtcccctccaccccagggacaggattggtgacagaaaa

SEQ ID NO: 174 - DNA, 80 bp, synthetic construct
  misc_feature 1..80: note = Target oligonucleotide sequence
  source 1..80: mol_type = other DNA
agggccggttaatgtggctctggttctgggtacttttatctgtcccctccaccactagggacaggattggtgacagaaaa

SEQ ID NO: 175 - DNA, 53 bp, synthetic construct
  misc_feature 1..53: note = Target oligonucleotide sequence
  source 1..53: mol_type = other DNA
cccacagtggggccactagggacaggattggtgacagaaaagccccatacccc

SEQ ID NO: 176 - DNA, 22 bp, synthetic construct
  misc_feature 1..22: note = Target oligonucleotide sequence
  source 1..22: mol_type = other DNA
cccacagtggggccactacccc

SEQ ID NO: 177 - DNA, 96 bp, synthetic construct
  misc_feature 1..96: note = Target oligonucleotide sequence
  source 1..96: mol_type = other DNA
cccacagtggggccactagtagaaaagccccatccttaggcctcccccatccttaggcctcctccttcctagtctcctgatattgggtctaacccc

SEQ ID NO: 178 - DNA, 94 bp, synthetic construct
  misc_feature 1..94: note = Target oligonucleotide sequence
  source 1..94: mol_type = other DNA
cccacagtggggccactagggacaggattggtgacagaaaagccccatccttaggcctcctccttcctagtctcctgatattgggtctaacccc

SEQ ID NO: 179 - DNA, 62 bp, synthetic construct
  misc_feature 1..62: note = Target oligonucleotide sequence
  source 1..62: mol_type = other DNA
cccacagtggggccacccttaggcctcctccttcctagtctcctgatattgggtctaacccc

SEQ ID NO: 180 - DNA, 38 bp, synthetic construct
  misc_feature 1..38: note = Target oligonucleotide sequence
  source 1..38: mol_type = other DNA
cccacagtggggccactagtgatattgggtctaacccc

SEQ ID NO: 181 - DNA, 94 bp, synthetic construct
  misc_feature 1..94: note = target oligonucleotide sequence
  source 1..94: mol_type = other DNA
cccacagtggggccactagggacaggattggtgacaaaaaagccccatccttacgcctcctccttcctagtctcctgatattgggtctaacccc

SEQ ID NO: 182 - DNA, 65 bp, synthetic construct
  misc_feature 1..65: note = Target oligonucleotide sequence
  source 1..65: mol_type = other DNA
cccacagtggggccactagggacaggcctcctccttcctagtctcctgatattgggtctaacccc

SEQ ID NO: 183 - DNA, 102 bp, synthetic construct
  misc_feature 1..102: note = Target oligonucleotide sequence
  source 1..102: mol_type = other DNA
cccacagtggggccactagggacagggggacaggattggtgacagaaaagccccatccttaggcctcctccttcctagtctcctgatattgggtctaacccc

SEQ ID NO: 184 - DNA, 76 bp, synthetic construct
  misc_feature 1..76: note = Target oligonucleotide sequence
  source 1..76: mol_type = other DNA
cccacaggattggtgacagaaaagccccatccttaggcctcctccttcctagtctcctgatattgggtctaacccc

SEQ ID NO: 185 - DNA, 23 bp, synthetic construct
  misc_feature 1..23: note = Target oligonucleotide
  source 1..23: mol_type = other DNA
ggggccactagggacaggatggg

SEQ ID NO: 186 - DNA, 23 bp, synthetic construct
  misc_feature 1..23: note = Target oligonucleotide
  source 1..23: mol_type = other DNA
gagatgatcgccccttcttctgg

SEQ ID NO: 187 - DNA, 75 bp, synthetic construct
  misc_feature 1..75: note = Target oligonucleotide sequence
  source 1..75: mol_type = other DNA
gggatcctgtgtccccgagctgggaccaccttatattcccagggtcggttaatgtggctctggttctgggtactt